SEQUENCE LISTING (115 sequences)

SEQ ID NO: 1 - DNA, 15275 bp, Artificial sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcgcggccgcgctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcactagtcattaagaaggaacttcagagttaagatgatcaagagtctcagcgaaagactgccaagcagcctcagtatcagaagttctttgatgttctcctctgaaccattcaacaaactcagcaacatcaagcaaagaccatctcaatctgtaaagagccaaagcagctggatcaggtgtatgtccagtaagttctgtgtatctagcaagagcagcaggatcatcagaaatcaaagaaagatctctttctgggatagcaagaccaacagtatcccaatcaaccaaaacataaccatcctctcccaagataagatttccaggatgtggctctccatgagtaacaaccaaaggagcacctcttcctctaactctagcaaccaattcaccgaactcagccattcttcctctcaaagtagcctcatgttcagccaaaagttctctagcaggttcagagaatggtccacctgtccaagtaccagaaggttcagcaagagcagtatgaactccatcaagaccagttggaaccatatcagttgtaggagtacacttaggaggagcttgtccatgaagctcagccaaaagaacaagaacttgatctctctctctttctgtaagtttctgaccgaactctcctggtctagcagaaacatgtgggaaaacagtcaaagcgtatctagaatcaagaggaaccaaagaagctccatcagatgtagttctaggagcaacaacgaaaggaagacctccttgctctctcaaatgaacagctgtatccatagctcttctaagacctctcaaagcagcaggagcaccatgtccacaatgttccttatgctccaaatcagaaactgtagcgaaccatctttgtccatcatctcctgtgatatgccaatggtaatctccgaaaccaagaggagcgtaagaagcagaagttgtagagattccgaactctctcaatccttggaaaagagactcctgatccaagttttcaggaagatcctccatgcaggctgtagccactgatgctgaaatcctaaaggaacaaaacttttgcataaaaattgaatcctttttcaaaaccaacatagaatttgctgaatttttcagctttttagatccaaaaacaagaaaacttgagcttttaggtacttggggcttatggaagttggaattgggattaagtgtctggatgccttgagccatgttgttaatctgtgccatggtggtgtgctgaggacctgcaaagttgaaaaccacaaacgaatcagaatgatgaaatttaacagctatccgactgaattaagtacatctcaagaacagatctgattaatttcagggaaaaaaatacataatcttcggtcaggattaaccaaacaaggcggttgttaatggatctgaattttttttttctttaaaataagaagactgttaacggatctgaaaattttcttaaaaagaaaacaattaacgtaataaagaactacagagaaaaattcaagacgtgttattgtaaaagagaaagaaaagattctgacgatcgatgaaactgtacaaaccctatcttaaactcaacaagctaatcgaaaacatgatcattcacgggactaatgaataacatagacaaaataaaatagacgaaccttgaggggagaggtagggaactgcgcaaagagaatgaagtagtttgttttgaattgaactgtgaagagagacagagggtgtgggcctttttatagctaaaggggtggaatcctctccaatcacattgtgacgcgtgtccttgtgtccctatttggtgcttattacacgggtgatatgtggtgtctgatgcgttttcggagtcctttcttggaaattcaggattgcaatttgcggggtggttagcgggttggtaatgttgacggagtcagacgcgcctacgtcagcttaatctgttacaacggcgttattaaattggaccccacttcacaatttgggccggaaccactgcaagtggagcgttctcagttttgtatgggcccctctatccactacaccaacggttggctcctattttacatgattaagaaatctattgcaaagctgtgatgtttgattttttaatggactaatgaatgattggaaagctttatcatttatttttttaataaggaaatgtcttgtaatcatccacgctttaactatttaaacaaatcatgaaacttaaaggtgacggatttaccaataaaataagggtataggacagcttaattgaaaaaacaaaaatgatatagtaaagaaaacttaagagcgatgggcatccttatcatctaatatgaataagctacaccatctgatctgcagtggacaagttgtcagattatacagttgttgatggctttaagaagcataattgtattattaatatttttatatataagtgcagctctattgtttatactactaatattctcgttttgcgttttatttaataattaattgtatgtactttataaaagaaaaatatcataaaatgagaaaaaaaaacaaaaattgactgttatatttgagtgtttttttacttatatgtattatttatatgtattatgctttaatataatatttttattttactaatatagacatatcaacgcgtgtttcaaactgattttttccctctcatctagatggtgttaatgttccttttatcatcaattaaacttgttgagaaacttatttgttatattaggtatgcaatataatattcgtataaattaaaaaacaaaaaattttgagagttgtttttaaaaattgatatatgggtgagtattaaagtttaaatatgtttaagaaatcattctaagatagaatatgcgcgatgaataaataaaatacctattttttaaaaatatgatatgctattttgtagaatacataaaagatgttttgtatgcgctaacaaaaagggtttttgggaaacaaaacacatataatattatttaagatgtaaaaagtttttccagtttttttttttcaaaacataatacataattcattgatcaatataattacaccttagatgcattcgcgcctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattccttgtaccgaggcaatctcgattccgagtaatcccattcgccattcaggctgcgcaactgttgggaagggcgatccgtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgcatgtcaggctcccttatacacagccagtctgcaggtcgaatacacttgtaccgaggcaatctcgattccgacaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 2 - DNA, 4861 bp, Artificial sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctaccaattgttgaattttgatttgttgaagttggctggagatgttgaatctaatcctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcccaactcctcaactttgacctgctcaaattggcaggcgatgtcgaatcgaatccggggcctatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 3 - DNA, 597 bp, Artificial sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctc

SEQ ID NO: 4 - DNA, 1491 bp, Artificial sequence
  Synthetic
atggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctac

SEQ ID NO: 5 - DNA, 66 bp, Artificial Sequence
  Synthetic
ggaagcggagctactaacttcagcctgctgaagcaggctggagacgtggaggagaaccctggacct

SEQ ID NO: 6 - DNA, 825 bp, Artificial sequence
  Synthetic
atgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgcc

SEQ ID NO: 7 - DNA, 66 bp, Artificial sequence
  Synthetic
ggttcaggtgccactaatttttctctcttgaaacaggccggtgacgttgaagagaacccaggtcca

SEQ ID NO: 8 - DNA, 1509 bp, Artificial sequence
  Synthetic
atgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtga

SEQ ID NO: 9 - DNA, 297 bp, Artificial Sequence
  Synthetic
gttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 10 - DNA, 3325 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcctcagcacaccaccatggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgcatggaggatcttcctgaaaacttggatcaggagtctcttttccaaggattgagagagttcggaatctctacaacttctgcttcttacgctcctcttggtttcggagattaccattggcatatcacaggagatgatggacaaagatggttcgctacagtttctgatttggagcataaggaacattgtggacatggtgctcctgctgctttgagaggtcttagaagagctatggatacagctgttcatttgagagagcaaggaggtcttcctttcgttgttgctcctagaactacatctgatggagcttctttggttcctcttgattctagatacgctttgactgttttcccacatgtttctgctagaccaggagagttcggtcagaaacttacagaaagagagagagatcaagttcttgttcttttggctgagcttcatggacaagctcctcctaagtgtactcctacaactgatatggttccaactggtcttgatggagttcatactgctcttgctgaaccttctggtacttggacaggtggaccattctctgaacctgctagagaacttttggctgaacatgaggctactttgagaggaagaatggctgagttcggtgaattggttgctagagttagaggaagaggtgctcctttggttgttactcatggagagccacatcctggaaatcttatcttgggagaggatggttatgttttggttgattgggatactgttggtcttgctatcccagaaagagatctttctttgatttctgatgatcctgctgctcttgctagatacacagaacttactggacatacacctgatccagctgctttggctctttacagattgagatggtctttgcttgatgttgctgagtttgttgaatggttcagaggagaacatcaaagaacttctgatactgaggctgcttggcagtctttcgctgagactcttgatcatcttaactctgaagttccttcttaatgactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 11 - DNA, 1815 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggt

SEQ ID NO: 12 - DNA, 216 bp, Artificial Sequence
  Synthetic
atggcacagattaacaacatggctcaaggcatccagacacttaatcccaattccaacttccataagccccaagtacctaaaagctcaagttttcttgtttttggatctaaaaagctgaaaaattcagcaaattctatgttggttttgaaaaaggattcaatttttatgcaaaagttttgttcctttaggatttcagcatcagtggctacagcctgc

SEQ ID NO: 13 - DNA, 996 bp, Artificial Sequence
  Synthetic
atggaggatcttcctgaaaacttggatcaggagtctcttttccaaggattgagagagttcggaatctctacaacttctgcttcttacgctcctcttggtttcggagattaccattggcatatcacaggagatgatggacaaagatggttcgctacagtttctgatttggagcataaggaacattgtggacatggtgctcctgctgctttgagaggtcttagaagagctatggatacagctgttcatttgagagagcaaggaggtcttcctttcgttgttgctcctagaactacatctgatggagcttctttggttcctcttgattctagatacgctttgactgttttcccacatgtttctgctagaccaggagagttcggtcagaaacttacagaaagagagagagatcaagttcttgttcttttggctgagcttcatggacaagctcctcctaagtgtactcctacaactgatatggttccaactggtcttgatggagttcatactgctcttgctgaaccttctggtacttggacaggtggaccattctctgaacctgctagagaacttttggctgaacatgaggctactttgagaggaagaatggctgagttcggtgaattggttgctagagttagaggaagaggtgctcctttggttgttactcatggagagccacatcctggaaatcttatcttgggagaggatggttatgttttggttgattgggatactgttggtcttgctatcccagaaagagatctttctttgatttctgatgatcctgctgctcttgctagatacacagaacttactggacatacacctgatccagctgctttggctctttacagattgagatggtctttgcttgatgttgctgagtttgttgaatggttcagaggagaacatcaaagaacttctgatactgaggctgcttggcagtctttcgctgagactcttgatcatcttaactctgaagttccttcttaatga

SEQ ID NO: 14 - DNA, 280 bp, Artificial Sequence
  Synthetic
tcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcgcgcggtgtcatctatgttactagatcgggaattcgtaatcatggtcatagc

SEQ ID NO: 15 - DNA, 24 bp, Artificial Sequence
  Synthetic
tgacaggatatattggcgggtaaa

SEQ ID NO: 16 - DNA, 26 bp, Artificial Sequence
  Synthetic
tggcaggatatattgtggtgtaaaca

SEQ ID NO: 17 - DNA, 398 bp, Artificial Sequence
  Synthetic
gcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgg

SEQ ID NO: 18 - DNA, 122 bp, Artificial Sequence
  Synthetic
gcgcaacgcaattaatgtgagttagctcactcattaggcaccccaggctttacactttatgcttccggctcgtatgttgtgtggaattgtgagcggataacaatttcacacaggaaacagct

SEQ ID NO: 19 - DNA, 276 bp, Artificial Sequence
  Synthetic
atgaccatgattacgccaagcgcgcaattaaccctcactaaagggaacaaaagctggaggacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacggatcgcccttcccaacagttgcgcagcctgaatggcgaatgg

SEQ ID NO: 20 - DNA, 977 bp, Artificial Sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 21 - DNA, 182 bp, Artificial Sequence
  Synthetic
ggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataa

SEQ ID NO: 22 - DNA, 795 bp, Artificial Sequence
  Synthetic
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 23 - DNA, 16962 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctagcttggccgggtcccgggctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatcaaaggtagaagcagaaacttacctagcatggtggccactccaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggcccagtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattctaggtacctcgcgaatgcatctagatccaatgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaagagcgtaggtacctcgcgaatgcatctagatccaatgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgataaggagactgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaaaccaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 24 - DNA, 3414 bp, Artificial Sequence
  Synthetic
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 25 - DNA, 307 bp, Artificial Sequence
  Synthetic
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatct

SEQ ID NO: 26 - DNA, 480 bp, Artificial Sequence
  Synthetic
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaac

SEQ ID NO: 27 - DNA, 1530 bp, Artificial Sequence
  Synthetic
ttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggc

SEQ ID NO: 28 - DNA, 192 bp, Artificial Sequence
  Synthetic
ttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatc

SEQ ID NO: 29 - DNA, 21 bp, Artificial Sequence
  Synthetic
ggggatcctatcagccgttcc

SEQ ID NO: 30 - DNA, 603 bp, Artificial Sequence
  Synthetic
cagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 31 - DNA, 262 bp, Artificial Sequence
  Synthetic
cgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 32 - DNA, 3432 bp, Artificial Sequence
  Synthetic
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgataaggagactgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 33 - DNA, 498 bp, Artificial Sequence
  Synthetic
atgggcgatcctaaaaagaaacgtaaggtcatcgataaggagactgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaac

SEQ ID NO: 34 - DNA, 1530 bp, Artificial Sequence
  Synthetic
ttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggc

SEQ ID NO: 35 - DNA, 192 bp, Artificial Sequence; identical to SEQ ID NO: 28
  Synthetic

SEQ ID NO: 36 - DNA, 1711 bp, Artificial sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggcccagtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 37 - DNA, 795 bp, Artificial Sequence
  Synthetic
atgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggcccagtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaa

SEQ ID NO: 38 - DNA, 1661 bp, Artificial Sequence
  Synthetic
gtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctggagtggccaccatgctaggtaagtttctgcttctacctttgatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagcccgggacccggccaagctagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagt

SEQ ID NO: 39 - DNA, 422 bp, Artificial Sequence
  Synthetic
gtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 40 - DNA, 187 bp, Artificial Sequence
  Synthetic
gtaagtttctgcttctacctttgatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcag

SEQ ID NO: 41 - DNA, 825 bp, Artificial Sequence
  Synthetic
atgctagcccgggacccggccaagctagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 42 - DNA, 160 bp, Artificial sequence
  Synthetic
tttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagt

SEQ ID NO: 43 - DNA, 21688 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctcgcggccgcgctatgaccatgattacgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcactagtcattaagaaggaacttcagagttaagatgatcaagagtctcagcgaaagactgccaagcagcctcagtatcagaagttctttgatgttctcctctgaaccattcaacaaactcagcaacatcaagcaaagaccatctcaatctgtaaagagccaaagcagctggatcaggtgtatgtccagtaagttctgtgtatctagcaagagcagcaggatcatcagaaatcaaagaaagatctctttctgggatagcaagaccaacagtatcccaatcaaccaaaacataaccatcctctcccaagataagatttccaggatgtggctctccatgagtaacaaccaaaggagcacctcttcctctaactctagcaaccaattcaccgaactcagccattcttcctctcaaagtagcctcatgttcagccaaaagttctctagcaggttcagagaatggtccacctgtccaagtaccagaaggttcagcaagagcagtatgaactccatcaagaccagttggaaccatatcagttgtaggagtacacttaggaggagcttgtccatgaagctcagccaaaagaacaagaacttgatctctctctctttctgtaagtttctgaccgaactctcctggtctagcagaaacatgtgggaaaacagtcaaagcgtatctagaatcaagaggaaccaaagaagctccatcagatgtagttctaggagcaacaacgaaaggaagacctccttgctctctcaaatgaacagctgtatccatagctcttctaagacctctcaaagcagcaggagcaccatgtccacaatgttccttatgctccaaatcagaaactgtagcgaaccatctttgtccatcatctcctgtgatatgccaatggtaatctccgaaaccaagaggagcgtaagaagcagaagttgtagagattccgaactctctcaatccttggaaaagagactcctgatccaagttttcaggaagatcctccatgcaggctgtagccactgatgctgaaatcctaaaggaacaaaacttttgcataaaaattgaatcctttttcaaaaccaacatagaatttgctgaatttttcagctttttagatccaaaaacaagaaaacttgagcttttaggtacttggggcttatggaagttggaattgggattaagtgtctggatgccttgagccatgttgttaatctgtgccatggtggtgtgctgaggacctgcaaagttgaaaaccacaaacgaatcagaatgatgaaatttaacagctatccgactgaattaagtacatctcaagaacagatctgattaatttcagggaaaaaaatacataatcttcggtcaggattaaccaaacaaggcggttgttaatggatctgaattttttttttctttaaaataagaagactgttaacggatctgaaaattttcttaaaaagaaaacaattaacgtaataaagaactacagagaaaaattcaagacgtgttattgtaaaagagaaagaaaagattctgacgatcgatgaaactgtacaaaccctatcttaaactcaacaagctaatcgaaaacatgatcattcacgggactaatgaataacatagacaaaataaaatagacgaaccttgaggggagaggtagggaactgcgcaaagagaatgaagtagtttgttttgaattgaactgtgaagagagacagagggtgtgggcctttttatagctaaaggggtggaatcctctccaatcacattgtgacgcgtgtccttgtgtccctatttggtgcttattacacgggtgatatgtggtgtctgatgcgttttcggagtcctttcttggaaattcaggattgcaatttgcggggtggttagcgggttggtaatgttgacggagtcagacgcgcctacgtcagcttaatctgttacaacggcgttattaaattggaccccacttcacaatttgggccggaaccactgcaagtggagcgttctcagttttgtatgggcccctctatccactacaccaacggttggctcctattttacatgattaagaaatctattgcaaagctgtgatgtttgattttttaatggactaatgaatgattggaaagctttatcatttatttttttaataaggaaatgtcttgtaatcatccacgctttaactatttaaacaaatcatgaaacttaaaggtgacggatttaccaataaaataagggtataggacagcttaattgaaaaaacaaaaatgatatagtaaagaaaacttaagagcgatgggcatccttatcatctaatatgaataagctacaccatctgatctgcagtggacaagttgtcagattatacagttgttgatggctttaagaagcataattgtattattaatatttttatatataagtgcagctctattgtttatactactaatattctcgttttgcgttttatttaataattaattgtatgtactttataaaagaaaaatatcataaaatgagaaaaaaaaacaaaaattgactgttatatttgagtgtttttttacttatatgtattatttatatgtattatgctttaatataatatttttattttactaatatagacatatcaacgcgtgtttcaaactgattttttccctctcatctagatggtgttaatgttccttttatcatcaattaaacttgttgagaaacttatttgttatattaggtatgcaatataatattcgtataaattaaaaaacaaaaaattttgagagttgtttttaaaaattgatatatgggtgagtattaaagtttaaatatgtttaagaaatcattctaagatagaatatgcgcgatgaataaataaaatacctattttttaaaaatatgatatgctattttgtagaatacataaaagatgttttgtatgcgctaacaaaaagggtttttgggaaacaaaacacatataatattatttaagatgtaaaaagtttttccagtttttttttttcaaaacataatacataattcattgatcaatataattacaccttagatgcattcgcgcctgcaggtcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaacctagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacactcctgcaggatttgaaaaatctcagaattgagattctaggtacctcgcgacctgcagggcatctaaggtgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtggcgcgcccgcccttacctgctagtcatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaagagcgtaggtacctcgcgacctgcagggcatctaaggtgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtggcgcgcccgcccttacctgctagtcatgggcgatcctaaaaagaaacgtaaggtcatcgataaggagactgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaaaccaagctaagcttggcactgcgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 44 - DNA, 4930 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtggcgcgcccgcccttacctgctagtcatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 45 - DNA, 480 bp, Artificial sequence; identical to SEQ ID NO: 26
  Synthetic

SEQ ID NO: 46 - DNA, 1530 bp, Artificial Sequence; identical to SEQ ID NO: 27
  Synthetic

SEQ ID NO: 47 - DNA, 192 bp, Artificial sequence; identical to SEQ ID NO: 28
  Synthetic

SEQ ID NO: 48 - DNA, 4948 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtggcgcgcccgcccttacctgctagtcatgggcgatcctaaaaagaaacgtaaggtcatcgataaggagactgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattc

SEQ ID NO: 49 - DNA, 498 bp, Artificial sequence; identical to SEQ ID NO: 33
  Synthetic

SEQ ID NO: 50 - DNA, 1530 bp, Artificial sequence; identical to SEQ ID NO: 34
  Synthetic

SEQ ID NO: 51 - DNA, 192 bp, Artificial sequence; identical to SEQ ID NO: 28
  Synthetic

SEQ ID NO: 52 - DNA, 20597 bp, Artificial Sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctggtctcagttacaatttgagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctagtctttaaaggtcacccgggaaccgcggcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgttataacctttctcttcttcttaggagccatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgataacaacgttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaggagttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaccagttacgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatggtgaaggttattggaaggagatctcttggtgtgcaaaggattttcgatattggacttccacaagatcacaacttccttttggctaacggagcaattgctgctaactgcttcaacagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaactcactgagagacccgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 53 - DNA, 1692 bp, Artificial Sequence
  Synthetic
tcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcgcgatcgcacaccaccatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggtgacctttaaagactagggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacact

SEQ ID NO: 54 - DNA, 597 bp, Artificial Sequence; identical to SEQ ID NO: 3
  Synthetic

SEQ ID NO: 55 - DNA, 795 bp, Artificial Sequence
  Synthetic
atgaagactaatctttttctctttctcatcttttcacttctcctatcattatcctcggccgaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagcatgatgagctttaa

SEQ ID NO: 56 - DNA, 297 bp, Artificial Sequence; identical to SEQ ID NO: 9
  Synthetic

SEQ ID NO: 57 - DNA, 4701 bp, Artificial Sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 58 - DNA, 1815 bp, Artificial Sequence; identical to SEQ ID NO: 11
  Synthetic

SEQ ID NO: 59 - DNA, 480 bp, Artificial Sequence; identical to SEQ ID NO: 26
  Synthetic

SEQ ID NO: 60 - DNA, 1540 bp, Artificial Sequence
  Synthetic
ttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctc

SEQ ID NO: 61 - DNA, 571 bp, Artificial Sequence
  Synthetic
tcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggc

SEQ ID NO: 62 - DNA, 369 bp, Artificial Sequence
  Synthetic
tgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaag

SEQ ID NO: 63 - DNA, 279 bp, Artificial Sequence
  Synthetic
cgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 64 - DNA, 4701 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 65 - DNA, 1540 bp, Artificial sequence
  Synthetic
ttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctc

SEQ ID NO: 66 - DNA, 2952 bp, Artificial sequence
  Sythetic
cgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatggtgaaggttattggaaggagatctcttggtgtgcaaaggattttcgatattggacttccacaagatcacaacttccttttggctaacggagcaattgctgctaactgcttcaacagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 67 - DNA, 1887 bp, Artificial sequence
  Synthetic
cgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaac

SEQ ID NO: 68 - DNA, 366 bp, Artificial sequence
  Synthetic
atggatcctaaaaagaaacgtaaggtcatggtgaaggttattggaaggagatctcttggtgtgcaaaggattttcgatattggacttccacaagatcacaacttccttttggctaacggagcaattgctgctaactgcttcaacagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctac

SEQ ID NO: 69 - DNA, 105 bp, Artificial sequence
  Synthetic
gtgaaggttattggaaggagatctcttggtgtgcaaaggattttcgatattggacttccacaagatcacaacttccttttggctaacggagcaattgctgctaac

SEQ ID NO: 70 - DNA, 213 bp, Artificial sequence
  Synthetic
cagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctac

SEQ ID NO: 71 - DNA, 391 bp, Artificial sequence
  Synthetic
caccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 72 - DNA, 24 bp, Artificial sequence
  Synthetic
tgacaggatatattggcgggtaaa

SEQ ID NO: 73 - DNA, 26 bp, Artificial sequence
  Synthetic
tggcaggatatattgtggtgtaaaca

SEQ ID NO: 74 - DNA, 977 bp, Artificial sequence; identical to SEQ ID NO: 20
  Synthetic

SEQ ID NO: 75 - DNA, 182 bp, Artificial sequence; identical to SEQ ID NO: 21
  Synthetic

SEQ ID NO: 76 - DNA, 795 bp, Artificial sequence; identical to SEQ ID NO: 22
  Synthetic

SEQ ID NO: 77 - DNA, 20381 bp, Artificial sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctggtctcagttacaatttgagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctagtctttaaaggtcacccgggaaccgcggcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgttataacctttctcttcttcttaggagccatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgataacaacgttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaggagttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaccagttacgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaactcactgagagacccgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 78 - DNA, 4596 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 79 - DNA, 466 bp, Artificial sequence
  Synthetic
tcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggc

SEQ ID NO: 80 - DNA, 264 bp, Artificial sequence
  Synthetic
tgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaa

SEQ ID NO: 81 - DNA, 4596 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 82 - DNA, 2946 bp, Artificial sequence
  Synthetic
cgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 83 - DNA, 360 bp, Artificial sequence
  Synthetic
atggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctac

SEQ ID NO: 84 - DNA, 111 bp, Artificial sequence
  Synthetic
atgatgctcaagaagattcttaaaatcgaggaacttgacgaaagggagcttattgacatcgaggtaagtgggaaccacctgttctacgccaacgatatactgacacacaat

SEQ ID NO: 85 - DNA, 20597 bp, Artificial sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctggtctcagttacaatttgagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctagtctttaaaggtcacccgggaaccgcggcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgttataacctttctcttcttcttaggagccatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgataacaacgttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaggagttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaccagttacgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatggtgaaggttattggaaggagatctcttggtgtgcaaaggattttcgatattggacttccacaagatcacaacttccttttggctaacggagcaattgctgctaactgcttcaacagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaactcactgagagacccgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 86 - DNA, 4701 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 87 - DNA, 1540 bp, Artificial sequence
  Synthetic
ttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctc

SEQ ID NO: 88 - DNA, 571 bp, Artificial sequence; identical to SEQ ID NO: 61
  Synthetic

SEQ ID NO: 89 - DNA, 369 bp, Artificial sequence; identical to SEQ ID NO: 62
  Synthetic

SEQ ID NO: 90 - DNA, 4701 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcctttcattcggaaccgaaattttgaccgtggagtatggacctcttccaattggaaagattgtgtcagaagagattaactgttctgtttactcagtggatccagaaggaagagtgtacacccaagctattgcacagtggcatgatagaggagagcaagaagtgcttgagtacgaacttgaggatggttctgtgattagggctacttctgatcacaggttcttgaccactgattaccagttgcttgcaattgaggaaattttcgctaggcaattggatcttttgactcttgagaacattaagcaaactgaggaagctcttgataaccataggcttccatttcctttgcttgatgcaggaactattaagtgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 91 - DNA, 1540 bp, Artificial sequence
  Synthetic
ttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctc

SEQ ID NO: 92 - DNA, 20381 bp, Artificial sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctggtctcagttacaatttgagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctagtctttaaaggtcacccgggaaccgcggcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgttataacctttctcttcttcttaggagccatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgataacaacgttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaggagttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaccagttacgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaactcactgagagacccgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 93 - DNA, 4596 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 94 - DNA, 1540 bp, Artificial sequence; identical to SEQ ID NO: 87
  Synthetic

SEQ ID NO: 95 - DNA, 466 bp, Artificial sequence; identical to SEQ ID NO: 79
  Synthetic

SEQ ID NO: 96 - DNA, 264 bp, Artificial sequence; identical to SEQ ID NO: 80
  Synthetic

SEQ ID NO: 97 - DNA, 4596 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatctgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 98 - DNA, 1540 bp, Artificial sequence; identical to SEQ ID NO: 91
  Synthetic

SEQ ID NO: 99 - DNA, 20381 bp, Artificial sequence; identical to SEQ ID NO: 92
  Synthetic

SEQ ID NO: 100 - DNA, 4611 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 101 - DNA, 1540 bp, Artificial sequence; identical to SEQ ID NO: 60
  Synthetic

SEQ ID NO: 102 - DNA, 481 bp, Artificial sequence
  Synthetic
tcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggc

SEQ ID NO: 103 - DNA, 264 bp, Artificial sequence; identical to SEQ ID NO: 80
  Synthetic

SEQ ID NO: 104 - DNA, 4611 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 105 - DNA, 1540 bp, Artificial sequence; identical to SEQ ID NO: 65
  Synthetic

SEQ ID NO: 106 - DNA, 2961 bp, Artificial sequence
  Synthetic
cgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagctcctccgacgttagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 107 - DNA, 375 bp, Artificial seqeunce
  Synthetic
atggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagctcctccgacgttagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctac

SEQ ID NO: 108 - DNA, 20426 bp, Artificial sequence
  Synthetic
gcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgagctggtctcagttacaatttgagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaaccctagtctttaaaggtcacccgggaaccgcggcttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgttataacctttctcttcttcttaggagccatggtggtgtgcgatcgcgagaaatattggttagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatattttgatgataacaacgttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaggagttagtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaaccagttacgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgggaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcaggtaagacaaccacaccaccatggatcctaaaaagaaacgtaaggtcatgatgctcaagaagattcttaaaatcgaggaacttgacgaaagggagcttattgacatcgaggtaagtgggaaccacctgttctacgccaacgatatactgacacacaatagctcctccgacgttagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagccaaactcactgagagacccgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacc

SEQ ID NO: 109 - DNA, 4611 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 110 - DNA, 1540 bp, Artificial sequence; identical to SEQ ID NO: 87
  Synthetic

SEQ ID NO: 111 - DNA, 481 bp, Artificial sequence; identical to SEQ ID NO: 102
  Synthetic

SEQ ID NO: 112 - DNA, 4611 bp, Artificial sequence
  Synthetic
gtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcaggtcacaccaccatgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcaccaggagcggatattgcttggatttgaaaacacaagttcagaccccacaggggatgaaggagatttctaatatccaagtcggtgatctcgtattgagcaatactggatataatgaagtcctgaacgtttttccaaagtcaaaaaagaagagttacaaaataacactcgaagatggcaaagaaataatatgcagcgaagaacacctcttccctacccagactggtgagatgaacatatctggggggttgaaggaggggatgtgtctctatgtaaaggaatgataactcgagaagggcagacgggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 113 - DNA, 1540 bp, Artificial sequence; identical to SEQ ID NO: 91
  Synthetic

SEQ ID NO: 114 - DNA, 2961 bp, Artificial sequence; identical to SEQ ID NO: 106
  Synthetic

SEQ ID NO: 115 - DNA, 375 bp, Artificial sequence; identical to SEQ ID NO: 107
  Synthetic

(19) United States
(12) Patent Application Publication
(10) Pub. No.: US 2024/0376484 A1
(43) Pub. Date: Nov. 14, 2024

(54) TRANSFORMING CANNABACEAE CELLS

(71) Applicants: Cibus US LLC, San Diego, CA (US); CIBUS EUROPE B.V., Breda (NL)

(72) Inventors: Alexandre DA SILVA CONCEICAO, Roseville, MN (US); Brady KURTZ, Roseville, MN (US); Anders UPPGAARD, Roseville, MN (US)

(73) Assignee: Cibus US LLC, San Diego, CA (US)

(21) Appl. No.: 18/692,895

(22) PCT Filed: Jun. 13, 2022
(86) PCT No.: PCT/US2022/033275

§ 371 Date: Mar. 18, 2024

Related U.S. Application Data
(60) Provisional application No. 63/245,301, filed on Sep. 17, 2021.

(51) Int. Cl. C12N 15/82; A01H 6/28; C12N 5/04

(52) U.S. Cl. CPC C12N 15/8202; A01H 6/28; C12N 5/04; C12N 15/8209; C12N 2510/00

(57) ABSTRACT

An example method of the present disclosure comprises imbibing a Cannabaceae seed in a hydration solution, excising a subset of embryonic tissue from the imbibed Cannabaceae seed to extract a Cannabaceae meristematic region or embryonic axis (EA), and exposing the Cannabaceae meristematic region or EA to a heterologous nucleotide sequence to transform Cannabaceae cells of the Cannabaceae meristematic region or EA.

### INCORPORATION-BY-REFERENCE OF MATERIAL SUBMITTED ELECTRONICALLY

Incorporated by reference in its entirety is a computer-readable nucleotide/amino acid sequence listing, an ASCII text file which is 433 kb in size, submitted concurrently herewith, and identified as follows: “C1633116111_SequenceListing_ST25” and created on Jun. 13, 2022.

### BACKGROUND

Cannabaceae is a family of dicotyledonous flowering plants that includes various trees, erect herbs, and twining herbs. An example Cannabaceae plant includes a Cannabis plant, such as industrial hemp. Industrial hemp is a cannabis plant variety having less than 0.3% tetrahydrocannabinol (THC). Industrial hemp can be cultivated to produce fiber, grain, or non-intoxicating medicinal compounds, such as cannabidiol (CBD) and terpenes. Functional genomics and breeding can be used to provide new Cannabaceae cultivars with traits of interest. Stable transgenic lines are useful for functional genomics. Cannabaceae plants can be recalcitrant to transformation. There is a need to improve transformation and regeneration efficiency of Cannabaceae for genetic breeding and other applications.

### SUMMARY

The present disclosure features methods and materials for transforming Cannabaceae plant cells, such as Cannabaceae meristematic region or embryonic axis (EA), and regenerating the transformed cells into various differentiated plant tissues, part plants, and whole plants. Methods described herein include systematic design of genotype-specific transformation and regeneration systems. Further embodiments implement the genotype-specific transformation and regeneration systems to provide transformed Cannabaceae plants from clonal material derived from a Cannabaceae meristematic region or EA that has one or more traits of interest.

Some aspects of the present disclosure are directed to a method comprising imbibing a Cannabaceae seed in a hydration solution, excising a subset of embryonic tissue from the imbibed Cannabaceae seed to extract a Cannabaceae meristematic region or EA, and exposing the Cannabaceae meristematic region or EA to a heterologous nucleotide sequence to transform Cannabaceae cells of the Cannabaceae meristematic region or EA.

Some aspects are directed to a method comprising imbibing a Cannabaceae seed in a hydration solution, excising a subset of embryonic tissue from the imbibed Cannabaceae seed to extract a Cannabaceae meristematic region or EA, and exposing the Cannabaceae meristematic region or EA to a heterologous nucleotide sequence to transform Cannabaceae cells of the Cannabaceae meristematic region or EA.

In some aspects, excising the subset of embryonic tissue comprises removing a seed coat without removing either of the cotyledons of the imbibed Cannabaceae seed.

In some aspects, excising the subset of embryonic tissue comprises removing a seed coat and one of the cotyledons of the imbibed Cannabaceae seed.

In some aspects, excising the subset of embryonic tissue comprises removing a seed coat and cutting a radicle of the imbibed Cannabaceae seed.

In some aspects, excising the subset of embryonic tissue comprises removing a seed coat, both cotyledons, and leaf primordia of the imbibed Cannabaceae seed.

In some aspects, excising the subset of embryonic tissue comprises removing a seed coat, one of the cotyledons, and leaf primordia of the imbibed Cannabaceae seed.

In some aspects, the method further comprises regenerating tissue from the transformed Cannabaceae cells using a culture medium comprising thidiazuron (TDZ), the tissue comprising one or more of shoots, roots, root hair structures, and full plants.

In some aspects, regenerating the tissue comprises inducing formation of shoots from the transformed Cannabaceae cells using the culture medium.

In some aspects, the culture medium comprises between about 1 milligram (mg)/liter (L) and about 20 mg/L of TDZ.

In some aspects, the method further comprises screening the transformed Cannabaceae cells or tissue regenerated from the transformed Cannabaceae cells for expression of the heterologous nucleotide sequence using a selection agent to screen the transformed Cannabaceae cells or tissue regenerated from the transformed Cannabaceae cells, the selection agent being selected from kanamycin A (kan), g418, spectinomycin, and glyphosate.

In some aspects, exposing the Cannabaceae meristematic region or EA to the heterologous nucleotide sequence comprises contacting the Cannabaceae meristematic region or EA with a bacterium strain that carries the heterologous nucleotide sequence.

In some aspects, exposing the Cannabaceae meristematic region or EA to the heterologous nucleotide sequence comprises exposing the Cannabaceae meristematic region or EA to an infection medium comprising the bacterium strain that is transformed to carry the heterologous nucleotide sequence.

In some aspects, the method further comprises removing the infection medium, co-culturing the Cannabaceae meristematic region or EA for a threshold period of time, and culturing the Cannabaceae meristematic region or EA in a selection medium to select transformed Cannabaceae meristematic region or EA.

In some aspects, the infection medium comprises thidiazuron (TDZ), metolachlor, magnesium sulfate, Tween, acetosyringone (MTA), thiols, GA3, Gamborg's B5 vitamins, DKW salts, AB salts, glucose, Silwet L-77, and combinations thereof.

In some aspects, the heterologous nucleotide sequence encodes a rare-cutting endonuclease operably connected to a promoter and optionally, a screening marker.

Some aspects are directed to a method comprising imbibing a Cannabaceae seed in a hydration solution, excising a subset of embryonic tissue from the imbibed Cannabaceae seed to extract a Cannabaceae meristematic region or EA, exposing the Cannabaceae meristematic region or EA to a heterologous nucleotide sequence to transform Cannabaceae cells of the Cannabaceae meristematic region or EA, and regenerating tissue from the transformed Cannabaceae cells using a culture medium comprising thidiazuron (TDZ).

In some aspects, n the culture medium comprises a shoot inducing medium comprising between about 1 mg/L and about 20 mg/L of TDZ.

In some aspects, transforming the Cannabaceae cells comprises exposing the Cannabaceae meristematic region or EA to an infection medium comprising a bacterium strain that carries the heterologous nucleotide sequence and that comprises between about 0.1 mg/L and about 2 mg/L of TDZ.

In some aspects, regenerating the tissue comprises inducing formation of shoots by transferring and culturing the Cannabaceae meristematic region or EA in an shoot inducing medium (SIM) comprising the TDZ, and after culturing in the SIM, transferring and culturing the Cannabaceae meristematic region or EA in a shoot elongation medium (SEM).

In some aspects, regenerating the tissue further comprises performing recovery by transferring and culturing the Cannabaceae meristematic region or EA in a regeneration medium comprising between about 0.1 mg/L and about 10 mg/L TDZ after the exposure to the heterologous nucleotide sequence and prior to transferring to the SIM, wherein the SIM comprises between about 1 mg/L and about 20 mg/L TDZ.

In some aspects, regenerating the tissue comprises inducing formation of shoots from the transformed Cannabaceae cells using the culture medium comprising the TDZ and inducing roots from the formed shoots.

In some aspects, inducing roots from the formed shoots comprises screening the formed shoots for shoots of a minimum height, rooting selected shoots of the minimum height to induce primary roots, and transferring and rooting shoots with the induced primary roots to induce new primary root and root hair structure formation.

In some aspects, the method further comprises transferring selected shoots with primary roots and root hair structures to soil.

Some aspects are directed to a transformed Cannabaceae explant produced using any of the methods described herein.

### BRIEF DESCRIPTION OF DRAWINGS

Various example embodiments can be more completely understood in consideration of the following detailed description in connection with the accompanying drawings, in which:

FIG. 1 is a flow diagram illustrating an example method for transforming Cannabaceae cells from a Cannabaceae meristematic region or EA, consistent with the present disclosure.

FIG. 2 is a flow diagram illustrating another example method for transforming Cannabaceae cells from a Cannabaceae meristematic region or EA, consistent with the present disclosure.

FIG. 3 is a diagram illustrating an example scheme for regenerating transformed Cannabaceae tissue from transformed Cannabaceae cells of a Cannabaceae meristematic region or EA, consistent with the present disclosure.

FIG. 4 illustrates an example expression construct for delivery of a heterologous sequence to Cannabaceae cells of a Cannabaceae meristematic region or EA, consistent with the present disclosure.

FIGS. 5A-5I illustrate example expression constructs for transforming Cannabaceae cells from a Cannabaceae meristematic region or EA, consistent with the present disclosure.

FIGS. 6A-6B include example images from samples from Table 1, consistent with the present disclosure.

FIGS. 7A-7B are images of a cannabis seedling explant transformed with a bacterium strain, consistent with the present disclosure.

FIG. 8 is an image of a plant regenerated from the cannabis seedling explant transformed with the bacterium strain as shown by FIGS. 7A-7B, consistent with the present disclosure.

FIG. 9 is an image of PCR data from the regenerated plant shown in FIG. 8, consistent with the present disclosure.

FIGS. 10A-10B are images of a cannabis meristematic region transformed with a bacterium strain, consistent with the present disclosure.

FIG. 11 is an image of a cannabis seedling explant transiently transformed with a bacterium strain, consistent with the present disclosure.

FIGS. 12A-12C are images of a cannabis seedling explant stably transformed with a bacterium strain, consistent with the present disclosure.

FIGS. 13A-13B are images of a cannabis EA stably transformed with a bacterium strain, consistent with the present disclosure.

FIGS. 14A-14B are images of a cannabis seedling explant stably transformed with a bacterium strain, consistent with the present disclosure.

FIGS. 15A-15B are images of a cannabis seedling explant stably transformed with a bacterium strain, consistent with the present disclosure.

FIGS. 16A-16G are data results of experiments assessing the different culture media and light conditions, consistent with the present disclosure.

FIGS. 17A-17F are images of explants from experiments assessing the transformation of the explants with different culture media, consistent with the present disclosure.

FIGS. 18A-18I illustrate results of transforming explants using the different plasmid vectors, consistent with the present disclosure.

### DETAILED DESCRIPTION

Aspects of the present disclosure are directed to a variety of methods for producing transformed Cannabaceae cell lines, plants, and plant parts from a Cannabaceae seed, such as a meristematic region of a seed part or a Cannabaceae EA. These methods can include extracting a Cannabaceae meristematic region or EA from a Cannabaceae seed, and transforming cells of the Cannabaceae meristematic region or EA by exposure to a heterologous nucleotide sequence. The exposure can cause transformation of the cells and the transformed cells can be used to regenerate transformed Cannabaceae tissue, such as plant parts or whole plants. The plants, plant parts, and plant cells of the present disclosure can be used to produce specific Cannabaceae varieties. In some embodiments, the plants, plant parts and plant cells can be used to develop new varieties or hybrids with specific traits and/or phenotypes. While the present invention is not necessarily limited to such applications, various aspects of the invention may be appreciated through a discussion of various embodiments using this context.

Cannabaceae plants are used for a variety of purposes. For example, cannabis, a type of Cannabaceae plants, is a fast-growing plant, and can be used as a low cost source of food, building or clothing material, biomass, paint, paper, and other material sources, as well as for medicinal or recreational purposes. To provide particular traits and/or varieties, plants can be transformed using gene editing techniques in tissue cultures. However, Cannabaceae plant cells in tissue culture can be unresponsive or recalcitrant to produce embryogenic cells that go on to form clonal and intact plants. Embodiments of the present disclosure are directed to transforming Cannabaceae plant cells from a Cannabaceae meristematic region or EA, which can be regenerated to form transformed plant tissue, such as plant parts or whole plants.

In various embodiments, the Cannabaceae meristematic region or EA is prepared from a Cannabaceae seed. For example, a whole Cannabaceae seed can be sterilized and imbibed in a hydrating solution, and a subset of embryonic tissue can be excised from the imbibed Cannabaceae seed to extract the Cannabaceae meristematic region or EA. The Cannabaceae meristematic region or EA is then exposed to a heterologous nucleotide sequence to transform Cannabaceae cells of the Cannabaceae meristematic region or EA. The heterologous nucleotide sequence can encode a polypeptide and can cause a mutation in the Cannabaceae cells. The tissue can be regenerated from the transformed Cannabaceae cells that express the heterologous nucleotide sequence. In some embodiments, the regenerated tissue can include plant parts or full Cannabaceae plants that exhibit a particular trait caused by the transformation.

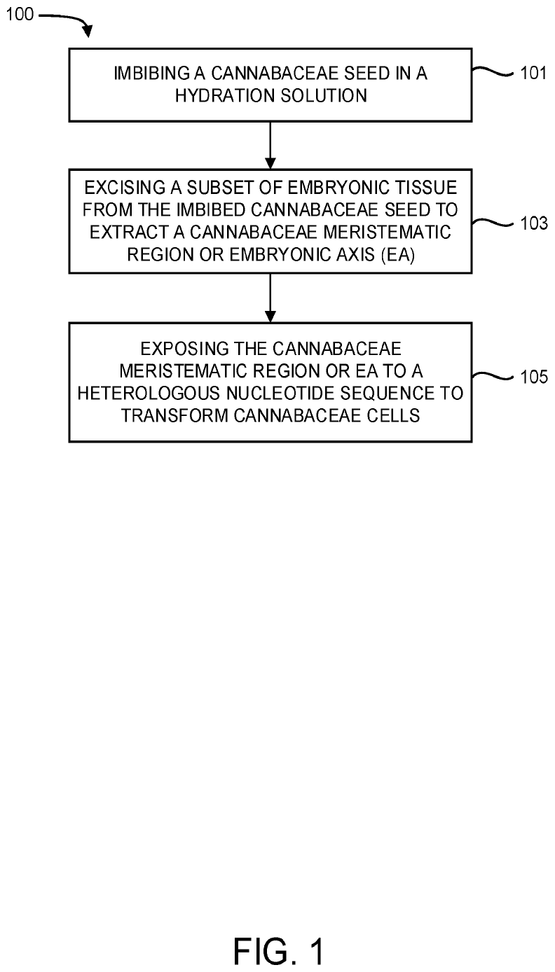

Turning now to the figures, FIG. 1 is a flow diagram illustrating an example method for transforming Cannabaceae cells from a Cannabaceae meristematic region or EA, consistent with the present disclosure. The method 100 can be used to regenerate transformed Cannabaceae tissue from the Cannabaceae cells transformed to express a heterologous nucleotide sequence.

At 101, the method includes imbibing a Cannabaceae seed in a hydration solution. In some embodiments, the seed is to full maturity, has an unblemished seed coat, and/or is free of bacteria, fungi or other pest vectors. For example, the hydration solution can include water, such as sterile distilled water or ddH₂O. In some embodiments, the Cannabaceae seed can be imbibed by exposing the Cannabaceae seed to the hydration solution for a period of time and/or using a rotary shaker. For example, the Cannabaceae seed can be placed in a 50 mL tube with sterile water and placed on a rotary shaker for between ten and twenty-four hours. In some embodiments, the Cannabaceae seed can be placed on a rotary shaker for between sixteen and twenty hours, between ten and fourteen hours, between twelve and fourteen hours, between fourteen and sixteen hours, between sixteen and eighteen hours, or between eighteen and twenty hours, among other ranges. In some embodiments, the Cannabaceae seed can be placed in a 100×25 mm petri dish with sterile filter paper and 2-3 mL of sterile water, sealed, and imbibed for between sixteen and twenty hours, between ten and fourteen hours, between twelve and fourteen hours, between fourteen and sixteen hours, between sixteen and eighteen hours, or between eighteen and twenty hours, among other ranges.

In some embodiments, prior to imbibing the Cannabaceae seed, the method 100 can include sterilizing the Cannabaceae seed. The Cannabaceae seed can be sterilized using scarification and hydrogen peroxide, in some embodiments. The scarification can be provided by exposing the Cannabaceae seed to an acid, such as sulfuric acid. In some embodiments, the Cannabaceae seed can be exposed to the acid for a period of 1 second to 30 seconds, and then the acid is removed, seed is rinsed with sterile water, and the Cannabaceae seed is exposed to hydrogen peroxide or another sterilization agent, for a period of between 1 to 30 minutes. However, examples are not so limited, and can include sterilizing the Cannabaceae seed using a sterilization solution including one or more sterilizing agents, such as a group consisting of ethanol, hypochlorite (NaClO or Ca(ClO)₂), benzalkonium chloride, silver nitrate, mercuric chloride and hydrogen peroxide. The sterilization solution can contain the sterilizing agent within a range of 0.01% to about 95% by volume. The Cannabaceae seed can be exposed to the sterilization solution for a period of 0.1 to about 30 minutes. The sterilization solution can further include a mild detergent such as a polysorbate (e.g., TWEEN 20 or TWEEN 80) or other non-ionic surfactant. In some embodiments, Cannabaceae seed can be washed with a sterilization solution including about 10% hydrogen peroxide. The Cannabaceae seed can be placed in a sterile 50 mL conical tube, and the sterilization solution added (e.g., by placing the 50 mL tube on a rotary shaker). After immersion in the solution, the Cannabaceae seed can be rinsed several times. For example, the sterilized Cannabaceae seed can be rinsed with sterile distilled water 3-5 times, for 1-10 minutes each rinse. After sterilizing, the Cannabaceae seed can be kept in distilled water in sealed Petri dishes in the laminar flow cabinet to prevent drying.

At 103, the method 100 includes excising a subset of embryonic tissue from the imbibed Cannabaceae seed to extract a Cannabaceae meristematic region or EA. As used herein, a “Cannabaceae EA” includes a portion of the seed between the plumule and radicle, not including the cotyledons. A “meristematic region” includes plant tissue containing undifferentiated cells (meristematic cells), found in zones of the plant where growth can take place. The Cannabaceae EA can include the plumule, radicle, and hypocotyl. The portion of the embryo between the cotyledon attachment point and the radicle is referred to as the hypocotyl. The EA terminates in a radicle, which is the region from which the root develops. After germination, the embryo can give origin to a seedling. In some embodiments, the undifferentiated cells of the meristematic region can have the ability to grow and regenerate into a plant (meristematic cells), found in zones of the plant where growth can take place.

In some embodiments, excising the subset of the embryonic tissue can include removing a seed coat without removing either of the cotyledons of the imbibed Cannabaceae seed. For example, the seed coat and the embryonic tissue can be removed using sterile forceps or other excision tools (e.g., scalpel, scissors). In some embodiments, excising the subset of the embryonic tissue includes removing a seed coat and one of the cotyledons of the imbibed Cannabaceae seed. Excising a subset of the embryonic tissue, such as one of the cotyledons, can reduce time and costs of extraction, and also reduce risk of destroying tissue, as compared to excising all the embryonic tissue. In some embodiments, excising the subset of the embryonic tissue includes removing a seed coat and cutting a radicle of the imbibed Cannabaceae seed. In some embodiments, excising the subset of the embryonic tissue includes removing a seed coat, both cotyledons, and leaf primordia of the imbibed Cannabaceae seed. In further embodiments, excising the subset of the embryonic tissue includes removing a seed coat, one of the cotyledons, and leaf primordia of the imbibed Cannabaceae seed. As noted above, removing one of the cotyledons can mitigate tissue damage, compared to excising all of the embryonic tissue, and thereby improving transformation efficiency and reducing time and costs associated with transformation. In various embodiments, the seed coat is removed using sterile forceps and then the embryos is ready for meristematic region or EA extraction. For example, the forceps can be used to hold the seed, and a scalpel blade can be used to slice off one or both of the cotyledons. Care can be taken to prevent or mitigate damage to the meristem. In some embodiments, after removing one or more cotyledons, the scalpel blade can be used to remove the leaf primordia, taking care to prevent or mitigate damage to the meristem.

At 105, the method 100 includes exposing the Cannabaceae meristematic region or EA to a heterologous nucleotide sequence to transform Cannabaceae cells of the Cannabaceae meristematic region or EA. The heterologous nucleotide sequence can encode a polypeptide to be expressed by and/or cause the transformation of Cannabaceae cells. For example, the heterologous sequence can encode a gene of interest. In response to expression of the heterologous nucleotide sequence, the transformed Cannabaceae cells can be used to regenerate tissue, plant parts, or whole plants that exhibit one or more traits associated with the gene of interest.

In some embodiments, the Cannabaceae cells are transformed using a bacterium-mediated transformation. For example, exposing the Cannabaceae meristematic region or EA to the heterologous nucleotide sequence can include contacting the Cannabaceae meristematic region or EA with a bacterium strain that carries the heterologous nucleotide sequence. The bacterium strain can be transformed to carry the heterologous nucleotide sequence. In some embodiments, the method includes transforming the Cannabaceae cells by exposing the Cannabaceae meristematic region or EA to an infection medium including the transformed bacterium strain, as further described herein.

The bacterium strain can include any strain that is transformed to induce expression of the heterologous nucleotide sequence, such as expressing a gene of interest. The bacterium strain can include a Rhizobia strain, such as a Rhizobium strain or Agrobacterium strain. In some embodiments, the bacterium strain includes Agrobacterium tumefaciens or a Rhizobium rhizogenes strain (R. rhizogenes), formerly known as Agrobacterium rhizogenes (A. rhizogenes). Rhizobium strains include a T-DNA which cause disease symptoms on infected plants and are contained on a Root Inducing (Ri) plasmid or a Tumor Inducing (Ti) plasmid. In some embodiments, a wild-type or disarmed strains can be used which harbor the Ti or Ri plasmids. For example, the Rhizobia strain can be disarmed in that the strain retains full vir gene functions to mediate T-DNA processing, transfer and integration but removes the oncogenes in the helper Ti or Ri plasmid. The bacteria strain includes a second T-DNA containing the gene of interest in a binary vector. During transformation, the virulence proteins encoded by the helper Ti plasmid act in trans to help T-DNA on the binary vector transfer from bacteria into plants. The T-DNA(s) from the bacterial strain can be stably integrated in the plant part. For example, tissue resulting from Rhizobia infection of plant tissue carry the T-DNA from the Ri or Ti plasmid and form vascular connections with their plant hosts.

Generally, the bacterium strain is prepared for infecting by introducing the heterologous nucleotide sequence into the bacterium strain (e.g., by electroporation) and culturing the transformed bacterium strain under conditions to select positively transformed cells. In some embodiments, the method 100 includes selecting the particular bacterium strain. Selection of an effective bacterium strain for the production of transformed cells can depend on the plant species to be infected and can be determined empirically.

In some embodiments, wild-type and disarmed strains of A. tumefaciens or A. rhizobium harboring Ti or Ri plasmids can be used for gene transfer into plants. Exemplary strains include but are not limited to A. tumefaciens derived from a nopaline-type strain, an octopine-type strain such as LBA4404, a succinamopine-type strain, for example, EHA101 or EHA105, and A. rhizogenes agropine-, mannopine- and cucumopine-type strains (e.g., MSU440, A13, 1855, 1193, A4, Qual, K599 (AKA NCPPB2659), and C58C1). In some cases, the A. tumefaciens strain is selected from the group consisting of AGL1, EHA105, GV3101, ICF320, CryX, LBA4404, C58, A136, A208, A348, Ach5, EHA101, NT1RE, NT1RE (pJK270), 1D1108, 1D1460, 1D1609, 1D132, 1D1478, and 1D1487, and the A. rhizobium strain is K599 or a strain derived from K599.

The nucleic acid sequence can be introduced by direct or indirect plant transformation methods. Embodiments of the present disclosure include the use of an activated Rhizobium bacterium to inject a piece of DNA into the Cannabaceae meristematic region or EA. The piece of DNA can be part of an expression cassette introduced into a binary plasmid for Rhizobium-mediated transformation. The expression cassette can be introduced to the bacterium using conventional methods.

The bacteria-mediated plant transformation includes the activation of the Rhizobium strain with DNA fragments cloned into plasmids. The activated Rhizobium strain is then used for transformation into individual plant cells of the meristematic region or EA. In some embodiments, the T-DNA includes the DNA sequence of a screening marker that confirms transformation and/or the location of transformants in the Cannabaceae meristematic region or EAs within cells. For example, a screening marker can include a gene that confers a selection marker or a label marker. The screening marker can be harbored on the same vector as the gene of interest or can be delivered as a separate vector. A label marker can confer a detectable label that can be visually, electrically, or otherwise detected, such as a fluorescent protein (a green fluorescent protein (GFP or eGFP), yellow fluorescent protein (YFP), or red fluorescent protein (RFP)) that can be detected using fluorescence microscopy. The selection marker can confer resistance to a toxic substance (e.g., a selection agent). Example selection markers for plant transformation include selectable markers conferring resistance to a toxic substance such as an antibiotic or herbicide. For example, the selection marker can include a kanamycin resistance gene, a g4198 resistance gene, a spectinomycin resistance gene, or a glyphosate resistance gene. Example label markers include β-glucuronidase (GUS), β-galactosidase, luciferase, Ruby, and chloramphenicol acetyltransferase. Screening markers can be useful to quantify or visualize the spatial pattern of expression of a gene in specific tissues and are frequently referred to as reporter genes because they can be fused to a gene or gene regulatory sequence for the investigation of gene expression. For example, the GFP mutant, Yellow Fluorescent Protein (YFP) can be used to indicate which plant cells the Rhizobium is able to integrate the foreign DNA.

In some embodiments, the heterologous nucleotide sequence includes gene editing reagents. The gene editing reagents can include rare-cutting endonuclease, or a portion (e.g., a subunit) thereof. The rare-cutting endonuclease can be a transcription activator-like effector nuclease (TALEN), a meganuclease, a zinc finger nuclease (ZFN), or a clustered regularly interspaced short palindromic repeats (CRISPR)/CRISPR-associated (Cas) nuclease reagent. For example, a rare-cutting endonuclease can be implemented as described in Baker, Nature Methods 9:23-26, 2012; Belahj et al., Plant Methods, 9:39, 2013; Gu et al., Nature, 435:1122-1125, 2005; Yang et al., Proc Natl Acad Sci USA, 103:10503-10508, 2006; Kay et al. Science, 318:648-651, 2007; Sugio et al., Proc Natl Acad Sci USA, 104:10720-10725, 2007; Römer et al. Science, 318:645-648, 2007; Schornack et al., J Plant Physiol, 163:256-272, 2006; and WO 2011/072246, which are each incorporated herein in their entireties for their teachings.

In some embodiments, the vector can include a TALEN sequence that encodes first and second TALEN and binding domains to bind to target sites and cause a mutation at the target sites. The first TALEN can generate a double stranded break at or near the first target site associated with a first binding domain and the second TALEN can generate a double stranded break at or near the second target site associated with a second binding domain. In some embodiments, the first and second binding domains can be associated with a target gene. In some embodiments, the TALEN sequence can be co-delivered to the plant tissue with the secondary transgene to cause expression of the secondary transgene along with the viral (e.g., hairy root) transgene.

As noted above, examples are not limited to TALENs and can include CRISPR/Cas systems (see, e.g., Belahj et al., Plant Methods, 9:39, 2013), among others or may not include the gene editing reagents. In some embodiments, a Cas9 endonuclease and a guide RNA can be used (either a complex between a CRISPR RNA (crRNA) and trans-activating crRNA (tracrRNA), or a synthetic fusion between the 3′ end of the crRNA and 5′ end of the tracrRNA (sgRNA)). The guide RNA directs Cas9 binding and DNA cleavage to homologous sequences that are adjacent to a proto-spacer adjacent motif (PAM). Once at the target DNA sequence, Cas9 generates a DNA double-strand break at a position three nucleotides from the 3′ end of the crRNA targeting sequence. In some embodiments, this approach or other approaches, such as ZFN and/or meganucleases, can be used in addition to TALE nucleases to obtain modified plant parts.

In various embodiments, the bacterium strain can form part of an infection medium used to infect and transform the Cannabaceae meristematic region or EA and associated cells. The infection medium can include the transformed bacterium strain, basal salts, sugar, and growth hormones and/or plant growth regulators. A plant growth regulator refers to or includes a compound that modifies plant growth, such as inducing regeneration. In some embodiments, the plant growth regulator includes thidiazuron (TDZ). For example, the infection medium can include about 1 milligram (mg)/liter (L) of TDZ. However embodiments are not so limited and can include other growth hormones or regulators and/or other concentrations of TDZ, such as meta-topolin, metolachlor, or magnesium sulfate, among others. In some embodiments, the infection medium includes magnesium sulfate, Tween, and acetosyringone (MTA). In some embodiments, the infection medium TDZ, metolachlor, magnesium sulfate, Tween, acetosyringone (MTA), thiols (e.g., DTT, STS, L-cysteine), GA3, Gamborg's B5 vitamins, DKW salts, AB salts, glucose, and combinations thereof. In some embodiments, the infection medium includes between about 0.1 mg/L and about 2 mg/L of TDZ. In some embodiments, the infection medium includes between about 0.1 mg/L and about 1.5 mg/L, about 0.1 mg/L and about 1.0 mg/L, about 0.1 mg/L and about 0.5 mg/L, about 0.5 mg/L and about 2 mg/L, about 1.0 mg/L and about 2 mg/L, about 1.5 mg/L and about 2 mg/L, about 0.5 mg/L and about 1.5 mg/L, about 0.5 mg/L and about 1.0 mg/L, about 1.0 mg/L and about 1.5 mg/L of TDZ, among other ranges.

Exposing the meristematic region or EA to the bacterium strain can include introducing the bacterium strain into the intracellular space of the cells of the meristematic region or EA. For example, the bacterium strain carrying the nucleic acid sequence can be introduced into the intracellular space by soaking the Cannabaceae meristematic region or EA with bacterium suspension in the infection medium. In some embodiments, the meristematic region or EA can be subjected to a chemical treatment that renders the cell walls more permeable (e.g., treatment with macerating enzymes such as cellulase, pectinase or macerozyme). The transformation efficiency can be enhanced by subjecting the donor material to vacuum infiltration, heat shock and/or centrifugation, and sonication.

In various embodiments, the transformation can include preparing a bacterium culture, inoculating, and co-cultivating. For example, the bacterium can be prepared by being cultured and suspended in an infection medium at a target concentration for inoculation of the Cannabaceae meristematic region or EA. The bacterium culture can be inoculated from a streaked plate, and the bacterial cells are washed and resuspended in a culture medium suitable for inoculation of the Cannabaceae meristematic region or EA. In some embodiments, the bacterium can be grown in a nutrient-rich liquid medium for about 8 hours as an initial starter culture, and the starter culture can be used to inoculate shake flasks containing an Agrobacterium minimal growth (AB) medium and grown for about 20 to 24 hours.

The density of the bacterium culture used for inoculation and the ratio of bacterium cells to the Cannabaceae meristematic region or EA can vary. For example, the concentration of bacterium in the infection medium can be varied to prevent overgrowth. Optimizing bacterium density can facilitate transformation. The bacterial density can be varied within the range at OD₆₀₀ (nm) between 0.4 and 0.9 which corresponds to 10,000,000 to 1,000,000,000 cfu/mL. In some embodiments, the culture is grown or diluted to a bacterial density of at least 0.7 to 1.5.

The infection medium can be supplemented with one or more compounds to improve infection and transformation rates by mitigating oxidative stress and/or bacterial overgrowth. For example, the infection medium can include an effective amount of an antioxidant, such as a thiol or related sulfur-containing compound. Suitable thiol compounds include L-cysteine, dithiothreitol (DTT), and sodium thiosulfate. The infection medium can include other chemicals (e.g., TDZ, meta-topolin, glyphosate), as described above. The infection medium can also include compounds that increase Agrobacterium transformation efficiency, such as acetosyringone.

In some embodiments, the method 100 can further include screening the transformed Cannabaceae cells, such as using the screening marker as previously described. Such embodiments include use of a selection protocol to select Cannabaceae meristematic regions, EAs, and/or cells that express one or more proteins encoded by the heterologous nucleotide sequence. As previously described, the heterologous nucleotide sequence can optionally encode a screening marker. The screening marker can be used to identify transformed Cannabaceae meristematic regions, EAs, and/or cells, and/or to differentiate between transformed and untransformed Cannabaceae meristematic regions, EAs, and/or cells. For example, selection for phosphomannose isomerase (PMI) expression includes the addition of mannose to the media with the restriction of other carbon sources. Transformed cells and/or tissue can be identified by various methods, some of which are described below.

As noted above, the screening marker can include a label marker and/or a selection marker. A label marker allows for visual, electrical, or other identification of the label. Examples of label markers include YFP, RFP, a betalain, and PDS editing that causes bleaching. Usually expression occurs spontaneously. In some embodiments, expression of the nucleotide sequence is induced by a change in biotic or abiotic factors, for example. Expression of the gene associated with the label marker can be measured by reverse transcription-polymerase chain reaction (RT-PCR), quantitative real-time polymerase chain reaction (qPCR), Northern blotting, dot-blot hybridization, in situ hybridization, nuclear run-on and/or nuclear run-off, RNase protection, or immunological and enzymatic methods such as ELISA, radioimmunoassay, and western blotting. Tissue can be assayed for expression of the label marker. Expression of a fluorescent protein marker can be visualized by UV excitation, fluorescence microscopy or flow cytometry. Transformation efficiency can be calculated and compared to conventional methods.

In some embodiments, a selection agent can be used to neutralize (e.g., kill) Cannabaceae cells and/or tissue which are not transformed to express the selection marker. For example, the selection marker can be associated with a gene that confers resistance to the selection agent, which can be a toxic substance such as an antibiotic or herbicide. In some embodiments, the method 100 includes using the selection agent to screen the transformed Cannabaceae cells or tissue regenerated from the transformed Cannabaceae cells. For example, the selection marker can include or be associated with an antibiotic, such as kanamycin, and can provide resistance to the kanamycin. If exposed to sufficient amounts of kanamycin, untransformed Cannabaceae meristematic regions and/or EAs are killed and transformed Cannabaceae meristematic region and/or EA can survive. Example selection agents include kanamycin A (kan), g418, spectinomycin, and glyphosate.

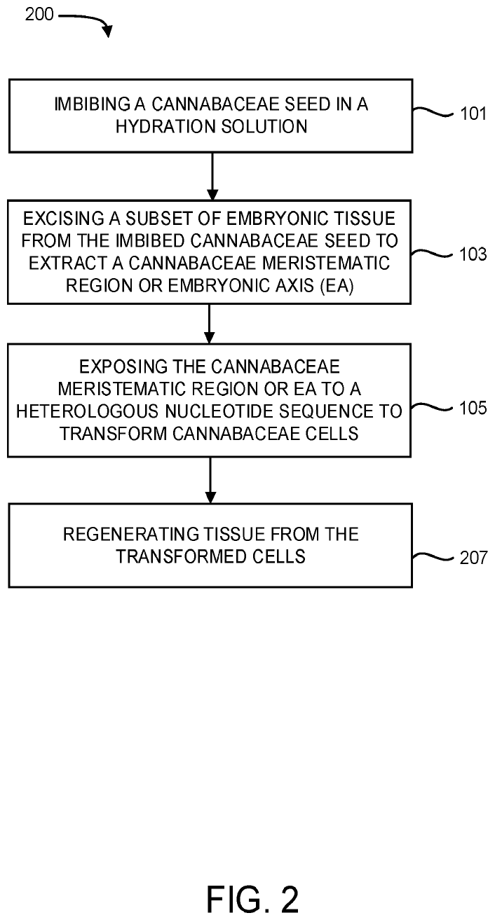

The method 100 can include Cannabaceae meristematic region and/or EA preparation, at 101 and 103, transformation of Cannabaceae cells, at 105, and regeneration of tissue from the transformed Cannabaceae cells, as further illustrated and described by FIG. 2. Preparation can include imbibing the Cannabaceae seed and removing subsets of embryonic material to produce the Cannabaceae meristematic region or EA. The transformation of Cannabaceae cells can include preparing an bacterium culture, inoculating, and co-cultivating. After co-cultivating, tissue can be regenerated. Regenerating tissue can include inducing shoot formation from the infected Cannabaceae meristematic region or EA, elongating the shoots, and inducing root formation from the shoots by rooting the shoots. The method 100 can provide transformed Cannabaceae plant parts, within 2-15 weeks after the Cannabaceae meristematic region or EA is exposed to the heterologous nucleotide sequence. The materials and methods of the present disclosure allow development of an efficient transformation system for Cannabaceae.

Inoculation can be performed at a temperature of about 20-28 degrees Celsius (° C.), about 23-28° C., from about 24-26° C. or at about 25° C. The duration of contact with the suspension can range from less than about 1 minute (a brief dip) to about 3 hours. After inoculation, excess bacterium suspension can be removed (e.g., by blotting or rinsing in sterile ddH₂O) and the EA is plated onto a co-cultivation medium.

Different plant tissue culture media can be used for the co-cultivation step. Co-cultivation of the Cannabaceae meristematic region or EA and the bacterium under in vitro conditions can be optimized with respect to duration, temperature, irradiance, and/or medium composition and pH. In other embodiments, a sterile filter paper wetted with sterile water can be used for the co-cultivation step. In some embodiments, the Cannabaceae meristematic region or EA us co-cultured for a threshold period of time. For instance, 1-4 days of co-cultivation can be sufficient for successful transformation, but longer periods (e.g., 5-7 days) can be utilized for recalcitrant genotypes in need of increased transformation efficiency. The temperature for incubation can range between 18-25° C., or 20-23° C., such as about 23° C. The co-culture can be performed in light or in light-limiting conditions. Lighting conditions can be optimized for plant genotype. In some embodiments, co-cultivation is carried out in the ambient light at 23±1° C. for two to four-day co-cultivation.

After co-cultivation, in some embodiments, the transformed meristematic regions and/or EAs can be rinsed in an antibiotic solution to remove excess bacterium. An antibiotic rinse solution can consist of one of more antibiotics such as cefotaxime, timentin, or carbenicillin. After the antibiotic rinse, the transformed meristematic region and/or EAs can be plated on an a selection medium (e.g., a SIM). The selection medium can include basal salts and an agent to inhibit bacterium growth, such as an antibiotic (e.g., carbenicillin, ticarcillin, clavulanic acid, ampicillin, spectinomycin, and/or cefotaxime). An example selection medium can include basal salts (e.g., DKW) and vitamins (e.g., B5 Vitamins), and an effective amount of the following: a sugar (e.g., sucrose, glucose, maltose), buffer to maintain pH of within a range of about 5.4 to 6 (e.g., 2-(N-morpholino) ethanesulfonic acid (MES)) and antibiotic agent(s) (e.g., Cefotaxime and a combination of ticarcillin and clavulanic acid (Timentin) and/or spectinomycin). In some embodiments, selection medium can further include a selection agent. For example, when the selection marker is spCN, a spectinomycin resistance gene, the selection medium can include an effective amount of spectinomycin (e.g., up to 150 mg/L).

In some embodiments, a recovery step is performed by transferring the transformed meristematic regions and/or EAs to a regeneration medium, such as a CL medium, to help reduce or eliminate contamination. The CL medium can include a biocide, such as Plant Preservation Mixture (PPM) which reduces or eliminates contaminants. The regeneration medium can further include vitamins, sugars, basal salts, and/or a plant growth regulator, such as TDZ. In some embodiments, the CL medium includes between about 0.1 mg/L and about 10 mg/L TDZ. In some embodiments, the CL medium includes about 1 mg/L TDZ or about 2 mg/L TDZ. In some embodiments, the CL medium includes between about 0.1 and about 9 mg/L, about 0.1 and about 8 mg/L, about 0.1 and about 7 mg/L, about 0.1 and about 6 mg/L, about 0.1 and about 5 mg/L, about 0.1 and about 4 mg/L, about 0.1 and about 3 mg/L, about 0.1 and about 2.5 mg/L, about 0.1 and about 2 mg/L, about 0.1 and about 1.5 mg/L, about 0.1 and about 1.0 mg/L, about 0.1 and about 0.5 mg/L, 0.5 and about 10 mg/L, about 1 and about 10 mg/L, about 1.5 and about 10 mg/L, about 2.0 and about 10 mg/L, about 2.5 and about 10 mg/L, about 2 and about 8 mg/L, about 2 and about 6 mg/L, about 2 and about 4 mg/L, about 2 and about 3 mg/L TDZ, among other ranges.

FIG. 2 is a flow diagram illustrating another example method for transforming Cannabaceae cells from a Cannabaceae meristematic region or EA, consistent with the present disclosure. The method 200 includes the steps 101, 103, 105 described by FIG. 1, the details of which are not repeated.

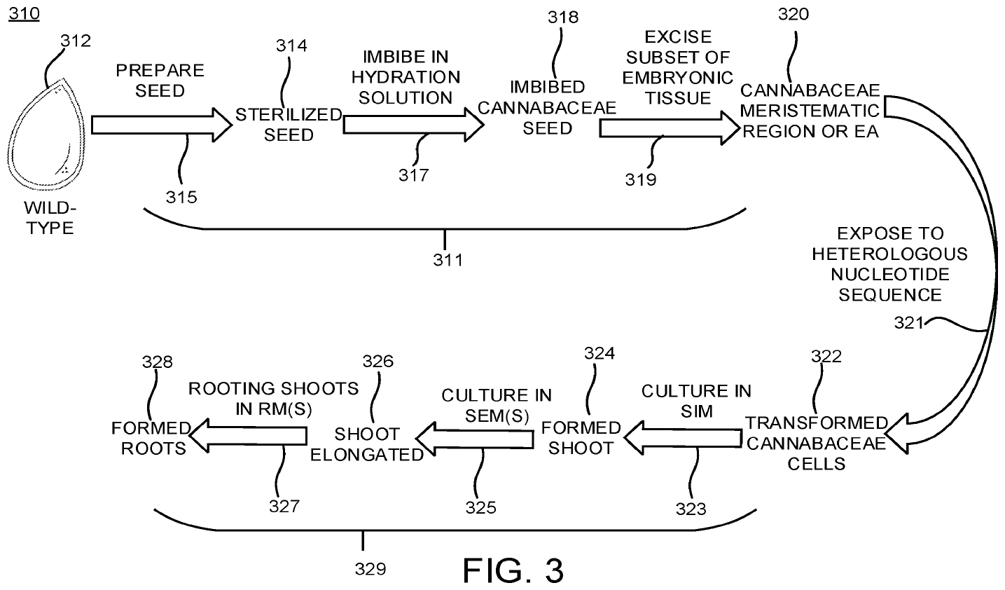

The method 200 further includes, at 207, regenerating tissue from the transformed Cannabaceae cells, the tissue including one or more of shoots, roots, root hair structures, and full plants. As further illustrated by FIG. 3, regenerating tissue can include inducing formation of shoots from the transformed Cannabaceae cells. For example, the induction of shoot formation can include inducing shoot formation and inducing shoot elongation. In some embodiments, regenerating the tissue can include inducing formation of shoots from the transformed Cannabaceae cells and inducing roots from the formed shoots.

In some embodiments, inducing shoot formation can include transferring and culturing the Cannabaceae meristematic region or EA in a shoot inducing medium (SIM), such as SIM+S100 (TDZ2) or (TDZ10) as further illustrated below. In some embodiments, the Cannabaceae meristematic region or EA can be cultured on SIM with 10 mg/L TDZ for between about 2 to about 6 weeks, and in some embodiments, between about 2 weeks and 3 weeks. The Cannabaceae meristematic region or EA can be oriented in the SIM with the radicle placed down in the SIM medium and the apical meristem placed up, similar to placing a plant with roots in the ground and such that the radicle can soak the SIM medium up. The SIM medium can include a plant growth regulator of TDZ. Somewhat surprisingly, a concentration of between about 0.1 mg/L and about 20 mg/L TDZ can be used in the SIM medium. In some embodiments, the SIM medium includes about 2 mg/L or about 10 mg/L, although embodiments are not so limited. The TDZ can be in the SIM in a concentration sufficient to break the apical dominance of the Cannabaceae meristem cells, thereby increasing transformation efficiency and resulting in more transgenic events. In some embodiments, the SIM medium can include about 0.1 mg/L and about 15 mg/L, 0.1 mg/L and about 10 mg/L, 0.1 mg/L and about 8 mg/L, 0.1 mg/L and about 6 mg/L, 0.1 mg/L and about 5 mg/L, 0.1 mg/L and about 4 mg/L, 0.1 mg/L and about 3 mg/L, 0.1 mg/L and about 2 mg/L, 0.1 mg/L and about 1 mg/L, 0.1 mg/L and about 0.5 mg/L, about 0.5 mg/L and about 20 mg/L, about 1 mg/L and about 20 mg/L, about 2 mg/L and about 20 mg/L, about 3 mg/L and about 20 mg/L, about 4 mg/L and about 20 mg/L, about 5 mg/L and about 20 mg/L, about 6 mg/L and about 20 mg/L, about 8 mg/L and about 20 mg/L, about 10 mg/L and about 20 mg/L, about 15 mg/L and about 20 mg/L, about 2 mg/L and about 15 mg/L, about 2 mg/L and about 10 mg/L, about 2 mg/L and about 5 mg/L TDZ, among other ranges.

After culturing in the SIM medium, the method 200 can include transferring and culturing the Cannabaceae meristematic region or EA in a first shoot elongation medium (SEM). In some embodiments, prior to transferring to the first SEM, the radicle of the shoots can be cut using a scalpel blade or other tool. After culturing in the first SEM, in some embodiments (although embodiments are not so limited), the method 200 can further include transferring and culturing the Cannabaceae meristematic region or EA in a second SEM. For both the first SEM and the second SEM, the radicle of the Cannabaceae meristematic region or EA is oriented down into the SEMs and the apical meristem placed up. The first SEM and second SEM can include different amounts of a selection agent, such as different amounts of spectinomycin.

In some embodiments, roots can be induced from the shoots. Inducing roots from the formed shoots can include screening the formed shoots for shoots of a minimum height, such as shoots that are one to two inches in height. Shoots of the minimum height can be selected and the method 100 further includes rooting the selected shoots to induce primary root formation, and transferring and rooting shoots with the induced primary shoots to induce new primary shoots and root hair structure formation. In various embodiments, the select shoots are rooted in a rooting medium (RM) and subcultured in fresh RM to induce the new primary roots and root hair structures. The method 200 can further include transferring selected shoots with primary roots and root hair structures to soil to regenerate partial or whole Cannabaceae plants that express the polypeptide, such as one or more proteins of interest.

The infection medium, co-cultivation medium, selection medium, regeneration medium (e.g., CL medium), SIM, first SEM, second SEM, and/or RM, as described above, generally comprise water, a basal salt mixture, a sugar, and one or more other components such as vitamins, selection agents, amino acids, and phytohormones. Each of the SIM, the first SEM, and the second SEM can include sugars, basal salts, growth hormones, and antibiotic agents, among other reagents, such as water and vitamins. For example, the SIM and SEMs can include nutritional sources of nitrogen, phosphorus, potassium, sulfur, calcium, magnesium, iron, boron, molybdenum, manganese, cobalt, zinc, copper, chlorine, and iodine. Macroelements can be provided as NH₄NO₃, (NH₄)₂SO₄, KNO₃, CaCl₂·2H₂O, MgSO₄·7H₂O, and KH₂PO₄. Micro elements can be provided as KI, H₃BO₃, MnSO₄·4H₂O, ZnSO₄, Na₂MoO₄ 2H₂O, CuSO₄·5H₂O, CoCl₂·6H₂O, CoSO₄·7H₂O, FeSO₄·7H₂O, and Na₂EDTA·2H₂O. Organic supplements such as nicotinic acid, Pyridoxine-HCl, Thiamine-HCl, and glycine can be included. Generally, the pH of the medium is adjusted to 5.7±0.5 using dilute KoH and/or HCl. Solid plant culture media can further include a gelling agent such as, for example, gelrite, agar or agarose. In various embodiments, as described in above example embodiments, the infection medium, the regeneration medium, and/or the SIM can include a plant growth regulator of TDZ. In some embodiments, the SIM can include a greater concentration of TDZ than the infection medium and/or the regeneration medium.

Any suitable plant culture medium can be used. Examples of media formulations include but are not limited to Murashige and Skoog (1962), N6, Linsmaier and Skoog (1965), L3 (Lin and Zhang (2005)), Uchimiya and Murashige (1962), Gamborg's media (1968), D medium, Nitsch and Nitsch (1969), DKW, and Schenk and Hildebrandt (1972).

The SIM and SEM(s) can include can include selection agents, phytohormones and/or plant growth regulators such as, for example, auxins, cytokinins, or gibberellins. The phytohormones can be selected from free and conjugated forms of naturally occurring phytohormones or plant growth regulators, or their synthetic analogues and precursors. Naturally occurring and synthetic analogues of auxins include, but are not limited to, indoleacetic acid (IAA), 3-indolebutyric acid (IBA), α-napthaleneacetic acid (NAA), 2,4-dichlorophenoxyacetic acid (2,4-D), 4-(2,4-dichlorophenoxy) butyric acid, 2,4,5-trichlorophenoxyacetic acid (2,4,5-T), 3-amino-2,5-dichlorobenzoic acid (chloramben), (4-chloro-2-methylphenoxy) acetic acid (MCPA), 4-(4-chloro-2-methylphenoxy) butanoic acid (MCPB), mecoprop, dicloprop, quinclorac, picloram, triclopyr, clopyralid, fluoroxypyr, dicamba and combinations thereof. Any combination of two or more auxins can be present in the nutritive media. Natural cytokinins and synthetic analogues of cytokinins include, but are not limited to, kinetin, zeatin, zeatin riboside, zeatin riboside phosphate, dihydrozeatin, isopentyl adenine 6-benzyladenine and combinations thereof. Any combinations of two or more cytokinins can be present in the mediums.

Presence of an effective amount of the auxin, and optionally an effective amount of the cytokinin, can promote cell division, improve regenerability, and/or induce the growth of more regenerative tissue. The effect of exogenous auxin to produce a morphological response can be enhanced by the addition of one or more antioxidants, amino acids, cobalt, or AgNO₃. Casamino acids provide a source of organic nitrogen in the form of amino acids hydrolyzed from Casein that can tolerate high salt conditions without degrading. Glutamine, asparagine, and methionine play complex roles in regulation of biosynthetic pathways that result in morphogenic response.

FIG. 3 is a diagram illustrating an example scheme for regenerating transformed Cannabaceae tissue from transformed Cannabaceae cells of a Cannabaceae meristematic region or EA, consistent with the present disclosure. The scheme 310 can include an implementation of the method 100 of FIG. 1 and/or the method 200 of FIG. 2.

At 315, Cannabaceae meristematic region or EA 320 can be prepared 311 by sterilizing the Cannabaceae seed 312 to generate a sterilized Cannabaceae seed 314. The sterilization can include the use of a sterilization agent, as previously described. The sterilized Cannabaceae seed 314 can be imbibed by soaking the Cannabaceae seed 312 in a hydration solution, at 317. A subset of the embryonic tissue can be excised from the imbibed Cannabaceae seed 318, at 319, to extract the Cannabaceae meristematic region or EA 320.

The Cannabaceae meristematic region or EA 320 can be exposed to the heterologous nucleotide sequence, at 321. In some embodiments, the exposure includes inoculating and co-culturing the Cannabaceae meristematic region or EA 320 with a bacterium strain by contacting the Cannabaceae meristematic region or EA 320 with an infection medium including the bacterium strain that carries the heterologous nucleotide sequence, and co-culturing under in vitro conditions sufficient for transformation, as previously described.

The exposure to the heterologous nucleotide sequence can cause transformation of Cannabaceae cells 322 within the Cannabaceae meristematic region or EA 320. In some embodiments, Cannabaceae cells can be screened to identify transformed Cannabaceae cells 322. Transformed cells or tissue can be screened by various methods. As described above, the cells or tissue can be screened using a selection agent in a selection medium and/or a screening marker expressed by transformed cells. In some instances, transfer of the nucleotide sequence is induced by a change in biotic or abiotic factors, for example. Expression of the gene of interest can be measured by RT-PCR, qPCR, Northern blotting, dot-blot hybridization, in situ hybridization, nuclear run-on and/or nuclear run-off, RNase protection, or immunological and enzymatic methods such as ELISA, radioimmunoassay, and western blotting. Tissue can be assayed for expression of a label. Expression of a fluorescent protein marker can be visualized by UV excitation, fluorescence microscopy or flow cytometry. Transformation efficiency can be calculated and compared to conventional methods. However embodiments are not so limited and in some embodiments, no screening may occur.

In some embodiments, at 323, the Cannabaceae meristematic region or EA with the transformed Cannabaceae cells 322 can be transferred (e.g., plated on) to a regeneration medium (e.g., CL medium) and cultured for a period of time, such as 1-5 days, to perform a recovery step. The regeneration medium (which may be interchangeably referred to as a recovery medium) can include the above-described CL medium containing a biocide, such as PPM. In some embodiments, the regeneration medium contains one or more selection agents and antibiotics. For example, the regeneration medium can contain reagents that neutralize untransformed cells and/or tissues and that neutralize the bacterium, while transformed cells and/or tissue can survive due to expression of the selection marker that is encoded by the heterologous nucleotide sequence.

After co-cultivation and recovery (with optional selection and/or screening), at 329, Cannabaceae meristematic region or EA with transformed Cannabaceae cells 322 can be used to regenerate tissue. For example, at 323, the Cannabaceae meristematic region or EA with transformed Cannabaceae cells 322 can be transferred to and cultured in a SIM for a period of time, such as 15-20 days, and under conditions to induce shoot formation. The conditions can include sterilization, white light, and 23° C. The SIM can include a reagent that neutralizes the bacterium strain among other reagents, such as TDZ, as previously described. In response, shoot formation can be induced. The formed shoots 324 can be removed from the SIM and the radicles of the shoots 324 can be cut. At 325, the formed shoots 324 with the cut radicles can be transferred to and cultured in an SEM(s) for a period of time, such as 30-50 days, and under conditions to induce elongated shoots 326. In some embodiments, the formed shoots 324 can be sub-cultured to fresh SEM every ten to fifteen days, such as every fourteen days of the period of time. The conditions can include sterilization, white light, and room temperature. In some embodiments, the formed shoots 324 with the cut radicles can be transferred to and cultured in a first SEM for a first period of time, such as 15-25 days, and then transferred to a second SEM for a second period of time, such as 15-25 days. For the second SEM, the formed shoots 324 can be sub-cultured in some embodiments. The elongated shoots 326 can be screened to selected shoots of a minimum height and the selected shoots can be rooted in a RM to induce formed roots 328, at 327. In some embodiments, rooting the elongated shoots 326 can include cutting the shoots and rooting the cut shoots on a RM until primary roots are developed that are a threshold length, such as developing at least two primary roots that are at least 1 cm in length (and for around 14 days). The rooted shoots can subcultured on fresh RM around every 7-14 days until new primary roots and root hair structures are formed. The rooted shoots 327 can be sent to acclimation in soil to form a partial or full Cannabaceae plant.

In various embodiments, the above-described methods and schemes can be used to obtain a Cannabaceae plant or plant part transformed to express the heterologous nucleotide sequence and/or a gene encoded thereby and that is produced using a method of any of the claims above. Some embodiments are directed to transformed Cannabaceae explant produced using a method of any of the claims above.

In some embodiments, a bacterium-mediated method can be used to obtain transformed Cannabaceae plant tissue from a Cannabaceae meristematic region or EA obtained from a Cannabaceae seed. The Cannabaceae meristematic region or EA can be obtained from any strain, species or cultivar of Cannabaceae that is of interest, without limitation. The methods of the present invention can be used with Cannabaceae meristematic region or EA of any Cannabaceae genotype including from hybrids and inbreds.

In some embodiments, a biolistics transformation method can be used to obtain transformed Cannabaceae plant tissue from a Cannabaceae meristematic region or EA. For example, the Cannabaceae meristematic region or EA can be modified by a bombardment technique, such as via particle bombardment with the expression construct. In some embodiments, the Cannabaceae meristematic region or EA can be exposed to the expression construct via bombardment using a bombardment gun and/or particles coated with the expression construct. Specific examples of bombardment driven transformation would be known to one of ordinary skill in the art.

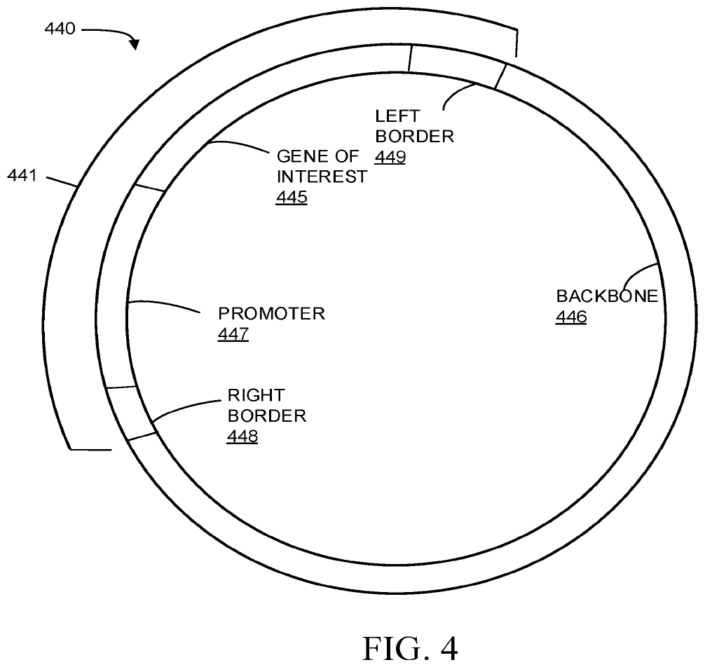

FIG. 4 illustrates an example expression construct for delivery of a heterologous sequence to Cannabaceae cells of a Cannabaceae meristematic region or EA, consistent with the present disclosure. The example expression construct 440 is or includes a binary vector containing an expression cassette 441 and a vector backbone 446. The expression cassette 441 includes a transgene that causes expression of a gene of interest 445. The transgene of the expression cassette 441 includes the gene of interest 445, a promoter 447, a left border 449, and a right border 448. In some embodiments, the left border 449 and the right border 448 can be separate from the expression cassette 441. The expression construct 440 and/or expression cassette 441 can include various additional components, such as TALE sequences, a screening marker, a terminator, and an additional expression cassette, among other components, such as signaling peptides.

The expression cassette 441 can include a gene encoding a foreign protein or a protein conferring an agronomic trait. Agronomic genes include genes that confer resistance to pests or disease, genes that confer resistance to an herbicide, and/or genes that confer or contribute to a value-added trait.

In some embodiments, a bacterium strain can be transformed using the expression construct 440. As used herein, an expression construct refers to or includes a nucleic acid sequence (e.g., DNA sequence) including one or more vectors or binary vectors carrying genes. A vector or binary vector includes or refers to a DNA sequence that includes one or more genes or transgenes, sometimes referred to as “inserts”, and a backbone. The vector or binary vector can include an expression cassette 441 that includes the gene or transgene and a regulatory sequence to be expressed by a transformed plant cell. Successful transformation can result in the expression cassette 441 directing plant cells to express one or more proteins of interest and/or otherwise express a target mutation.

The expression cassette 441 can include the heterologous sequence encoding the gene of interest 445, T-DNA border sequences 448, 449, and a promoter 447. Expression cassettes typically include a promoter operably linked to a nucleotide sequence encoding the gene of interest 445, which is optionally operably linked to termination signals and/or other regulatory elements. For example, the expression cassette 441 can include TALEN T-DNA. The expression cassette 441 can also include sequences required for proper translation of the nucleotide sequence, post-translational processing, localization and accumulation in a cellular compartment or tissue, or secretion into the tissue culture media. As an example, the gene of interest 445 can be associated with a protein comprising signal peptides of plant origin (e.g., the N-terminal signal peptide from the tobacco PR1a protein or calreticulin) or signal peptides from eukaryotic secreted polypeptides, e.g., mammalian signal peptides, can be efficiently secreted through the plasma membrane and cell wall into the extracellular medium. In some embodiments, the heterologous nucleotide sequence encoding the gene of interest 445 includes an N-terminal tag. For example, in the case of membrane-spanning or -anchored proteins, the expression construct 440 can be prepared that modifies the N-terminus by replacing the membrane-spanning or membrane-anchoring domain with an N-terminal secretion signal sequence.

The expression cassette 441 comprising the nucleotide sequence can be chimeric, meaning that at least one of its components is heterologous with respect to at least one of its other components. In some embodiments, the expression cassette 441 can be one which is naturally occurring or assembled entirely extracellularly (e.g., by recombinant cloning techniques). An expression cassette 441 can be obtained by placing (or inserting) a promoter sequence upstream of an endogenous sequence, which thereby becomes functionally linked and controlled by the inserted promoter sequence.

In some embodiments, the promoter 447 can include an inducible promoter, a strong promoter, or a tissue specific promoter. For example, the heterologous nucleotide sequence encoding the gene of interest 445 can be operably connected to the inducible promoter, strong promoter, or tissue specific promoter. In some embodiments, the promoter 447 can include a constitutive promoter. An inducible promoter can be switched on and off, whereas a constitutive promoter can always be active. For example, the heterologous nucleotide sequence encoding the gene of interest 445 can be operably connected to an ubiquitin promoter (Ubi) or a 35S Cauliflower Mosaic Virus (CMV) promoter.

A promoter typically includes at least a core (basal) promoter but can also include at least one control element. Such elements include upstream activation regions (UARs) and, optionally, other DNA sequences that affect transcription of a nucleic acid, which can include synthetic upstream elements. Factors for selecting a promoter to drive expression of the copy include efficiency, selectability, inducibility, desired expression level, and cell- or tissue-type specificity. The promoter 447 can be one which preferentially expresses in Cannabaceae EA or under certain conditions, e.g., is a tissue specific promoter. The promoter 447 can be modulated by factors such as temperature, light or stress. For example, inducible promoters can be used to drive expression in response to external stimuli (e.g., exposure to an inducer). Suitable promoters include, but are not limited to, a light-inducible promoter from ssRUBISCO, MAS promoter, rice actin promoter, maize ubiquitin promoter, PR-I promoter, CZ19B1 promoter, milps promoter, CesA promoter, Gama-zein promoter, Glob-1 promoter, maize 15 kDa zein promoter, 22 kDa zein promoter, 27 kDa zein promoter, δ-zein promoter, waxy promoter, shrunken 1 promoter, shrunken 2 promoter, globulin 1 promoter, pEMU promoter, maize H3 histone promoter, beta-estradiol promoter, and dexamethasone-inducible promoters. Non-limiting examples of constitutive promoters include 35S promoter, such as 35S CMV promoter, 2×35S promoter, nopaline synthase (NOS) promoter, ubi3, among others.

A promoter for driving expression can have strong transcriptional activity. A strong promoter drives expression at a high level, or at about 1/10 transcripts to about 1/100 transcripts to about 1/1,000 transcripts. Enhancers can be used in combination with the promoter regions to increase transcription levels. When the gene of interest 445 is endogenous to the plant species, the expression cassette can be effective for achieving at least a 2-fold, 3-fold, 4-fold, 5-fold, 6-fold, 7-fold, 8-fold, 9-fold, or 10-fold increase in the level of expression compared to the expression level of the endogenous gene of interest in the wild type plant tissue.

The heterologous nucleotide sequence encoding the gene of interest 445 can include a DNA sequence derived from various organisms, including but not limited to, humans and other mammals and/or vertebrates, invertebrates, plants, sponges, bacteria, fungi, algae, and archaebacterial. The heterologous nucleotide sequence can encode a protein having at least 70% (e.g., at least 75%, 80%, 85%, 90%, 95%, 97%, 98%, 99%, or 100%) sequence identity to the amino acid sequence of a corresponding wild-type gene. In some cases, the heterologous nucleotide sequence has significant similarity and shared functional domains with the sequence encoding the protein. The heterologous nucleotide sequence can be obtained from a related organism having a homologous, orthologous, or paralogous gene to a gene encoding the protein. Methods for identifying conserved or similar heterologous nucleotide sequences and constructing recombinant genes encoding proteins, optionally with various modifications for improved expression (e.g., codon optimized sequences), include conventional techniques in molecular biology. For example, PCR amplification or design and synthesis of overlapping, complementary synthetic oligonucleotides can be annealed and ligated together to yield a gene with restriction sites for cloning, or subcloning from another already cloned source, or cloning from a library.

In some embodiments, the heterologous nucleotide sequence can include the sequence of a gene occurring in the wild-type Cannabaceae plant, or a sequence having a percent identity that allows it to retain the function of the gene encoded product, such as a sequence with at least 90% identity. This sequence can be obtained from the organism or organism part or can be synthetically produced. The sequence can have at least 91%, 92%, 93%, 94%, 95%, 96%, 97%, 98%, or 99% identity to the gene occurring in the wild-type organism. The sequence can be inserted at a different locus than that of the wild-type gene and be operably linked to a different promoter than the wild-type gene. The bacterium-mediated methods of the present disclosure do not depend on a particular expression construct. Any expression construct that can be introduced into a plant cell may be employed in the methods. The nucleic acid sequence can be part of an expression cassette introduced into a T-DNA region of a plasmid for bacterium-mediated transformation. The T-DNA can be present in a binary vector. The nucleotide sequence can include a DNA sequence of interest and other sequences such as regulatory sequences for expression of the DNA sequence of interest. Binary vectors usable in the invention are known to the skilled person. The binary vector typically has an antibiotic resistance gene for allowing selection in bacteria. For increasing transfection efficiency, the bacterium can harbor a virG gene. The virG can be present on the same plasmid as the heterologous nucleic acid sequence, or in a helper plasmid.

The methods of the present disclosure can include constructing an expression cassette and/or expression construct that functions in Cannabaceae plant cells. Construction can include selection of the various components required for introduction and expression by Cannabaceae plant cells. For example, the expression cassette can be incorporated into a recombinant, double-stranded plasmid or vector molecule including a promoter that functions in Cannabaceae plant cells to cause the production of an RNA sequence, (b) a structural DNA sequence that causes the production of an RNA sequence that encodes a desired polypeptide, and (c) a 3′ non-translated DNA sequence that functions in plant cells to cause the addition of polyadenylated nucleotides to the 3′ end of the RNA sequence. Methods for preparing plasmids or vectors containing desired components are well known in the art.

In some embodiments, the expression cassette includes sequences encoding a screening marker and associated regulatory elements as described, and also a nucleic acids sequence conferring a particular trait when expressed. These trait-conferring sequences include genes of agronomic interest (e.g., genes for insect or pest tolerance, environmental or stress tolerance, herbicide tolerance), genes for quality improvements such as nutritional enhancements, or any desirable changes in plant physiology, growth, development, morphology or plant product(s).

In some embodiments, the expression cassette can include a nucleotide sequence for producing targeted mutations within the Cannabaceae genome. For example, sequences encoding a rare-cutting endonuclease, or a portion (e.g., a subunit) thereof can be introduced by the expression cassette. The methods provided herein can include the transient expression of programmable RNA-guided endonucleases, or portions (e.g., subunits) thereof. The rare-cutting endonuclease can be a fusion protein that contains a DNA binding domain and a catalytic domain with cleavage activity. TALE-nucleases and ZFNs are examples of fusions of DNA binding domains with the catalytic domain of the endonuclease FokI. In other examples, the rare-cutting endonuclease is a meganuclease, such as a wild type or variant homing endonuclease.

In some embodiments, the Cannabaceae plants of the present disclosure can be used to produce new plant varieties. In some embodiments, the plants are used to develop new, unique and superior varieties or hybrids with specific traits and/or phenotypes.

The described methods can be used to produce a transgenic line. The transgenic line can be crossed, with another (non-transformed or transformed) line, to produce a new transgenic Cannabaceae line. Alternatively, a genetic trait that has been engineered into a particular Cannabaceae cultivar using the above-described techniques can be moved into another line using backcrossing techniques. For example, a backcrossing approach can be used to move an engineered trait from a public, non-elite inbred line into an elite inbred line, or from an inbred line containing a foreign gene in its genome into an inbred line or lines which do not contain the gene. “Crossing” can refer to a simple X by Y cross, or the process of backcrossing.

Embodiments of the methods provided herein can include removing the transgene to provide a new non-transgenic Cannabaceae plant. For example, genetic techniques can be used to provide progeny of a transformed plant with a transgene-induced deletion (e.g., targeted deletion induced by expression of a transgene expressing a rare cutting endonuclease) that lack the transgene. In example embodiments, progeny plants can be obtained by self-pollinating (selfing) a transformed Cannabaceae plant that is heterozygous for the transgene by segregation. Selfing of such heterozygous plants provides for the transgene to segregate out of a subset of the progeny plant population.

In some embodiments, a transformed Cannabaceae plant includes at least a first transgene, and which is otherwise capable of expressing all the physiological and morphological characteristics of the donor Cannabaceae plant. In other embodiments, a transformed Cannabaceae plant includes a cis-genic modification that alters expression of one or more genes compared with the mother Cannabaceae plant, while expressing a trait of the mother plant to the same extent as the mother plant, when grown under the same conditions. In other embodiments, a transformed Cannabaceae plant includes one or more targeted mutation (e.g., deletion) that alters the expression of one or more genes of the donor Cannabaceae plant. For example, transformed plant can exhibit lower levels of THC than the donor Cannabaceae plant as a result of the altered gene expression. In some embodiments, a Cannabaceae plant comprises a single locus conversion. The single locus conversion can include a dominant or recessive allele. The locus conversion can confer a trait upon the transformed Cannabaceae plant.

A Cannabaceae plant can include stacked traits that provide a combined effect, resulting from the use of multiple nucleic acid constructs or transformation events. For example, multiple constructs as described above can be introduced into Cannabaceae plant cells by the same or different methods, including the introduction of such a trait by the inclusion of two transcription cassettes in a single transformation vector, the simultaneous transformation of two expression constructs, retransformation using plant tissue expressing one construct with an expression construct for the second gene, or by crossing transgenic plants via traditional plant breeding methods, with the resulting product is a plant having both characteristics.

Plant parts can include products and compositions produced or purified from plants produced by the methods described herein, including the stalks, fibers, pulp, flowers, seeds, and the like. Products produced from Cannabaceae plants include industrial textiles, building materials, foods and nutritional supplements, personal care products such as soap, lotions, balms and the like, animal bedding, industrial products such as paints, inks, solvents and lubricants, consumer textiles, animal feed, etc. In some cases, the Cannabaceae plants and plant parts are used to provide extracts, which can be used as a flavoring or aromatic component, or for obtaining Cannabaceae-derived medicinal compounds.

Various terminology as used in the Specification (including claims) connotes a plain meaning in the art unless otherwise indicated. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not. The singular forms “a”, “and”, and “the” include plural references unless the context clearly dictates otherwise.

The various ranges provided herein include the stated range and any value or sub-range within the stated range. Furthermore, when “about” is utilized to describe a value or percentage this includes, refers to, and/or encompasses variations (up to +/−10%) from the stated value or percentage.

“Explant” refers to a plant part having regeneration potential via micropropagation. The explant can be capable of regenerating a shoot, root, or whole plant.

“Donor plant” refers to a source of explants. A donor Cannabaceae plant can be any type of Cannabaceae plant. In some cases, the donor plant is a female plant (e.g., not a hermaphrodite). In some embodiments, the donor plant is a Cannabaceae genotype that is susceptible to Rhizobium infection, and which exhibits a desired regeneration response. The donor Cannabaceae plant can be from an elite line having one or more desired traits.

“Transformation” refers to the transfer of a nucleotide sequence into a cell, and “genetic transformation” refers to the transfer and incorporation of DNA, especially recombinant DNA, into a cell. The term “transformant” refers to a cell, tissue or organism that has undergone transformation.

“Expression cassette” can refer to a DNA sequence capable of directing expression of a particular nucleotide sequence in an appropriate host cell, comprising a promoter operably linked to a nucleotide sequence of interest, which is optionally operably linked to termination signals and/or other regulatory elements. An expression cassette can also include sequences required for proper translation of the nucleotide sequence. The coding region can code for a protein of interest but can also code for a functional RNA of interest, for example antisense RNA or a non-translated RNA, in the sense or antisense direction. The expression cassette can be chimeric, meaning that at least one of its components is heterologous with respect to at least one of its other components. The expression cassette can be assembled using in part endogenous components. For example, the expression cassette can be obtained by placing (or inserting) a promoter sequence upstream of an endogenous sequence, which becomes functionally linked and controlled by the inserted promoter sequence.

As used herein, Cannabaceae refers to a plant of the family Cannabaceae. For example, the Cannabaceae plant or plant part can include a plant or plant part that belongs to the genus of Cannabis, sometimes referred to as a cannabis plant or plant part, and which includes Cannabis sativa, Cannabis indica, and Cannabis ruderalis). However, embodiments are not so limited, and the Cannabaceae plant or plant part can include Humulus (e.g., hops), Celtis, Alphananthe, Chaetachme, Gironniera, Lozanella, Parasponia, Pteroceltis, and/or Trema plants or plant parts, among other plants or plant parts. The term “plant” generally refers to whole plants, but when “plant” is used as an adjective, refers to any substance which is present in, obtained from, derived from, or related to a plant, such as plant organs (e.g., leaves, stems, roots, flowers), single cells (e.g., pollen), seeds, plant cells including tissue cultured cells, products produced from the plant. The term “Cannabaceae plant part” refers to one or more plant tissues or organs which are obtained from a whole plant of the family Cannabaceae. Cannabaceae plant parts include vegetative structures (for example, leaves, stems), roots (for example, hairy roots or non-hairy roots), floral organs/structures, seed (including embryo, endosperm, and seed coat), plant tissue (for example, vascular tissue, ground tissue, and the like), cells and progeny of the same.

A “Cannabaceae plant cell” is the structural and physiological unit of the plant, comprising a protoplast and a cell wall. Plant cells can be cells in culture. A Cannabaceae plant cell can be in the form of an isolated single cell or aggregate of cells such as a friable callus, or a cultured cell, or can be part of a higher organized unit, for example, a Cannabaceae plant tissue, plant organ, or plant. A Cannabaceae plant cell can be a protoplast, a gamete producing cell, or a cell or collection of cells that can regenerate into a whole plant. “Cannabaceae plant tissue” means differentiated tissue in a plant or obtained from a plant (“explant”) or undifferentiated tissue derived from immature or mature embryos, seeds, roots, shoots, fruits, pollen, and various forms of aggregations of plant cells in culture, such as calli. Plant tissues in or from seeds, such as Cannabaceae seeds, include a seed coat or testa, storage cotyledon, and embryo.

“Meristem” or “meristematic region” refers to a plant tissue containing undifferentiated cells (meristematic cells), found in zones of the plant where growth can take place. Meristematic cells give rise to organs of the plant and keep the plant growing.

The term “variety” or “cultivar” refers to a population of plants that share characteristics which separate them from other plants of the same species. While possessing one or more distinctive traits, a variety can be further characterized by a small overall variation between individuals within that variety. A “pure line” variety can be created by several generations of self-pollination and selection, or vegetative propagation from a single parent using tissue or cell culture techniques. A variety can be essentially derived from another line or variety. A variety is “essentially derived” from an initial variety if: (a) it is predominantly derived from the initial variety, or from a variety that is predominantly derived from the initial variety, while retaining the expression of the characteristics that result from the genotype or combination of genotypes of the initial variety; (b) it is distinguishable from the initial variety; and (c) except for the differences which result from the act of derivation, it conforms to the initial variety in the expression of the characteristics that result from the genotype or combination of genotypes of the initial variety. Essentially derived varieties can be obtained by the selection of a natural or induced mutant, a somaclonal variant, a variant individual from plants of the initial variety, backcrossing, or transformation. A “line” as distinguished from a variety can often denote a group of plants used non-commercially, such as for plant research. A line typically displays little variation between individuals for one or more traits of interest, although there can be some variation between individuals for other traits.

“Transformation frequency” refers to the percentage of plant cells that are successfully transformed with a heterologous nucleotide sequence after performance of a transformation protocol on the cells to introduce the nucleic acid. An increased “transformation efficiency” refers to an improvement, such as an increase in transformation frequency and quality events that impact overall efficiency of the transformation process by reducing resource use.

“Regeneration” refers to a morphogenic response that results in the production of new tissues, organs, embryos, whole plants or parts of whole plants that are derived from a single cell or a group of cells. The methods of the present disclosure, regeneration proceeds from the meristematic region or EA. “Regenerative capacity” refers to the ability of a plant cell to undergo regeneration. “Regeneration efficiency” can be calculated from the number of plantlets regenerated from embryogenic calli or from an individual explant.

The term “gene of interest” or “trait of interest” corresponds to a gene or trait expressed by transformed Cannabaceae cells using techniques as described herein.

Various embodiments are implemented in accordance with the underlying provisional application, U.S. Provisional Application No. 63/245,301, filed on Sep. 17, 2021, and entitled “Transformed Cannabaceae Cells and Methods Thereof”, to which benefit is claimed and which is fully incorporated herein by reference in its entirety for its teaching.

### Experimental Embodiments

Various experimental embodiments were directed to transforming Cannabaceae cells from a Cannabaceae meristematic region or EA. Such embodiments include preparing the Cannabaceae meristematic region and/or EA, transforming Cannabaceae cells of the Cannabaceae, and regenerating tissue for the transformed Cannabaceae cells. Some embodiments were directed to providing repeatable, reliable and simple transformation and regeneration system from Cannabaceae meristematic regions and/or EAs.

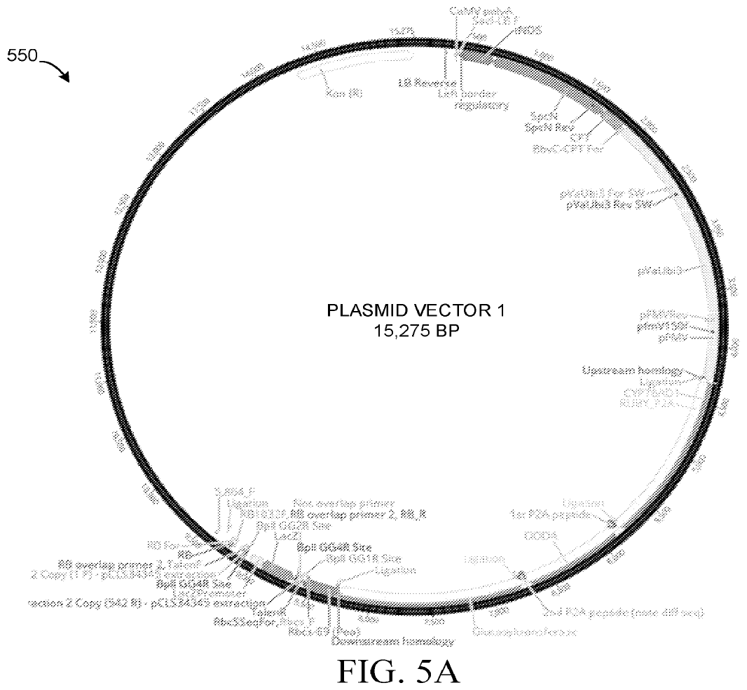

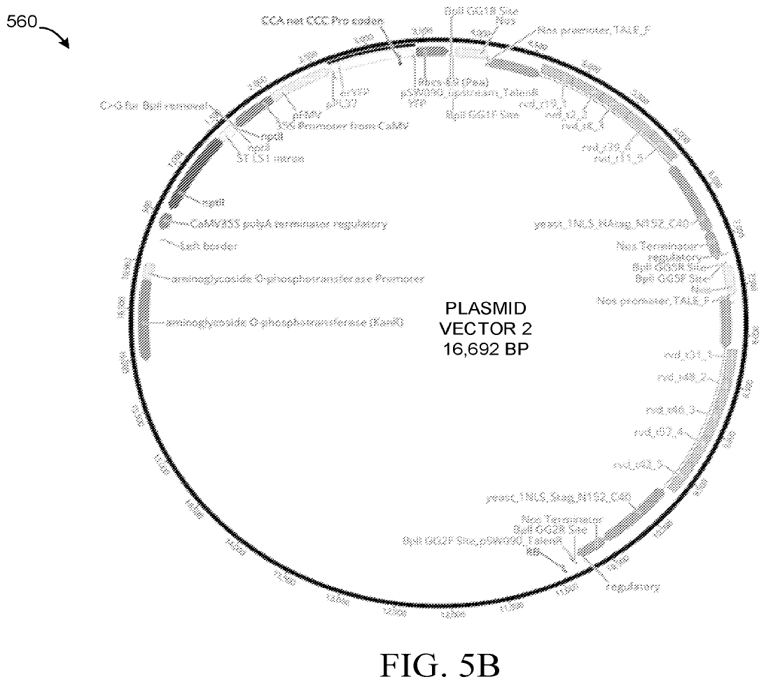

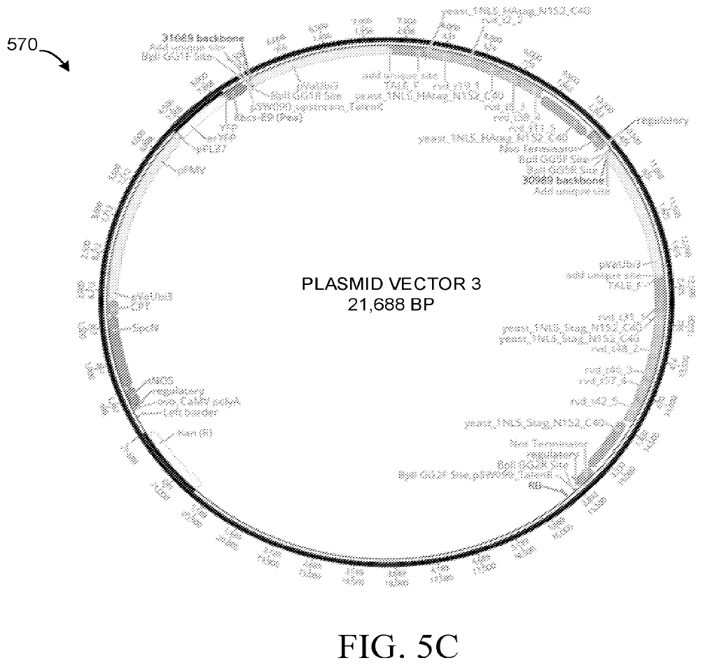

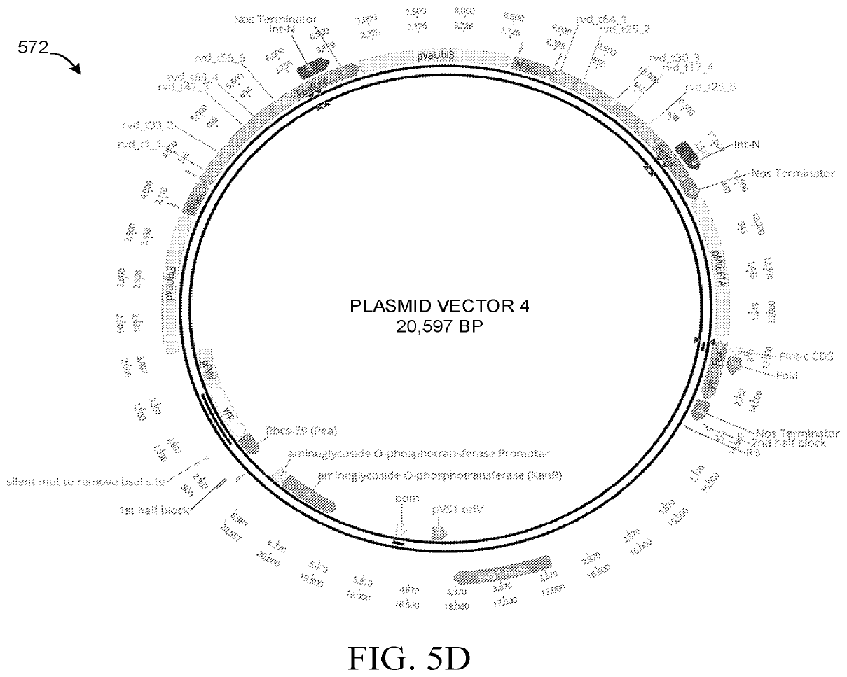

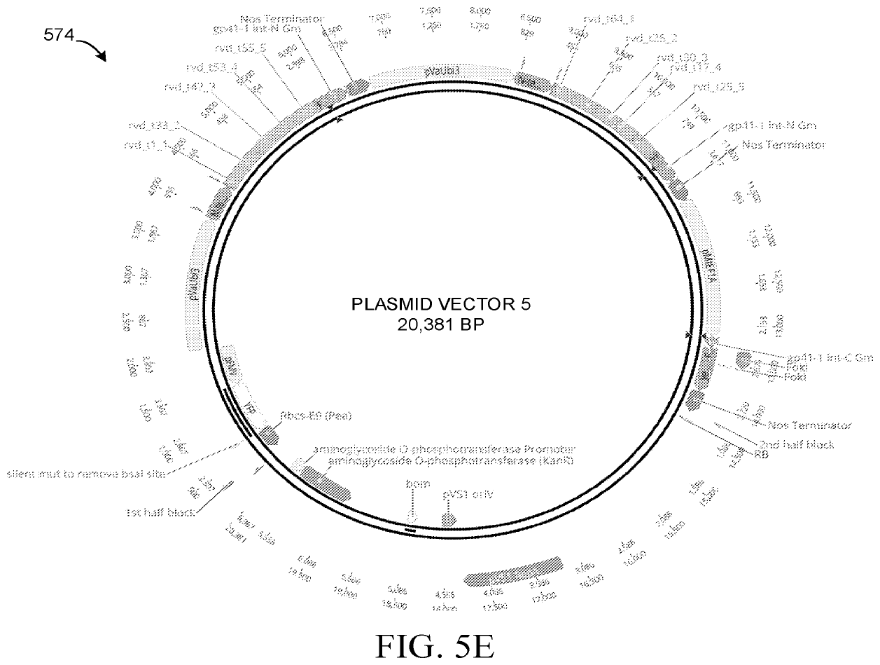

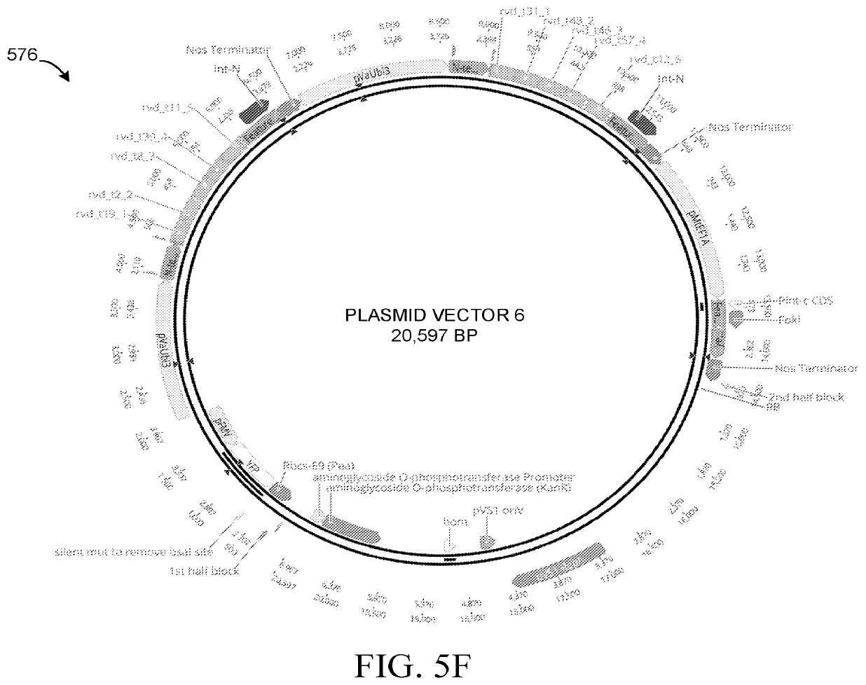

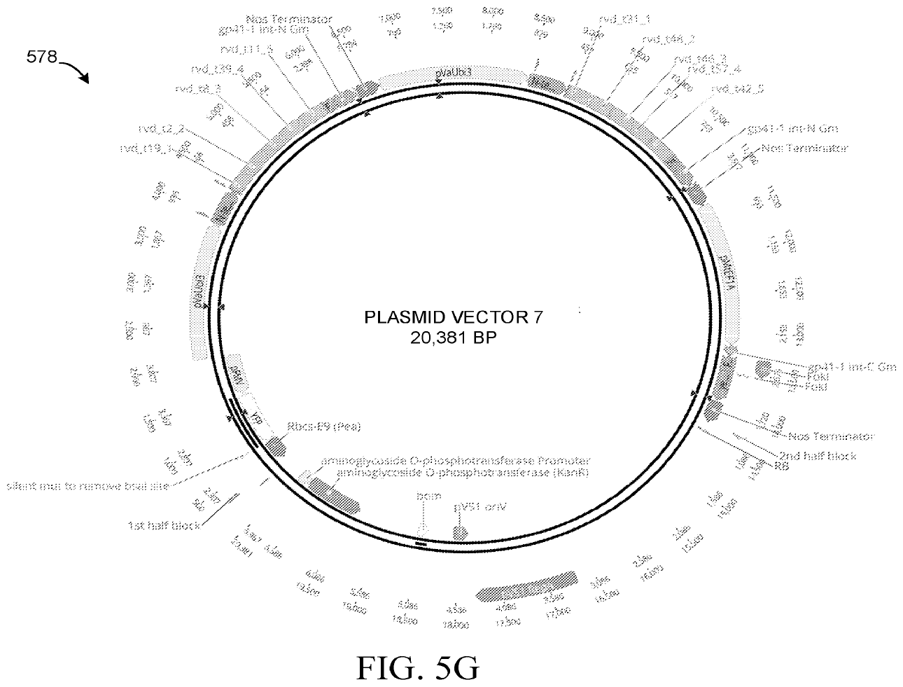

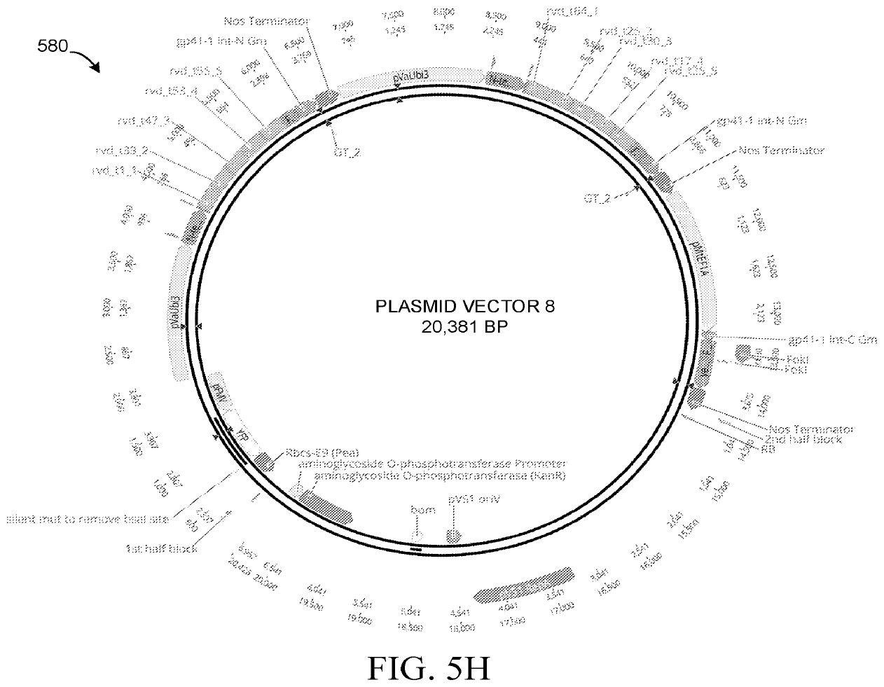

FIGS. 5A-5I illustrate example expression constructs for transforming Cannabaceae cells from a Cannabaceae meristematic region or EA, consistent with the present disclosure. In some experimental embodiments, the expression constructs were used for bacterium strain transformation (e.g., A. rhizogenes transformation) contains a right T-DNA border sequence and a left T-DNA border sequence, to allow the bacterium strain to deliver the DNA into the Cannabaceae plant cells. The expression constructs are plasmids and can be referred to as plasmid vectors. The expression constructs further include a DNA sequence coding the transgene, which is codon optimized according to the codon bias used by the target, and cloned in vectors, are under the regulation of a promoter, and a terminator. Constitutive promoters and root specific promoters are selected for tissue-specific approaches.

FIG. 5A illustrates an example plasmid vector 550 that includes a DNA sequence coding for at least one enzyme associated with the production of a betalain, sometimes herein referred to as the “betalain vector”. The plasmid vector 550 contains the gene encoding the enzymes CYP76AD1, DODA, and glucosyltransferase driven by the FMV promoter, with CYP76AD1 linked to DODA and DODA linked to glucosyltransferase by 2A self-cleaving peptides P2A, sometimes herein referred to as the “betalain cassette”. The plasmid vector 550 further includes a plant selectable marker cassette and a LacZ cassette, which are in reverse orientation on the plasmid, as further described below. The plant selectable marker cassette encodes a selection marker that when expressed, confers resistance to a selection agent (e.g., bacteria or other toxic substances) for selection of transformed plant cells, a promoter, and a terminator. The LacZ cassette encodes a LacZ gene and LacZ promoter used as a selection marker. The gene cassettes are flanked by the left border (LB) and right border (RB) T-DNA sequences allowing for transfer of the entire sequence or transgene into the Cannabaceae plant cells by the bacterium strain of A. rhizogenes, e.g., 18R12. The plasmid backbone also contains a bacterial selection marker cassette that encodes the kanamycin resistance (KanR) gene for selection and maintenance of the plasmid within the A. rhizogenes strain, and which is in reverse orientation on the plasmid. The Cannabaceae plant part was transformed using the 18R12 strain. The plasmid vector 550 sequence is illustrated by SEQ ID NO: 1. Further identified is the sequence of the betalain cassette (SEQ ID NO: 2), the plant selectable marker cassette (SEQ ID NO: 10), the T-DNA borders of the RB (SEQ ID NO: 15) and LB (SEQ ID NO: 16), the LacZ cassette (SEQ ID NO: 17) and the bacterial selection marker cassette (SEQ ID NO: 20). The betalain cassette (SEQ ID NO: 2) encodes the FMV promoter (SEQ ID NO: 3), CYP76AD1 (SEQ ID NO: 4), P2A 1 (SEQ ID NO: 5), DODA (SEQ ID NO: 6), P2A 2 (SEQ ID NO: 7), glucosyltransferase (SEQ ID NO: 8), and a rbcS terminator (SEQ ID NO: 9). The plant selectable marker cassette (SEQ ID NO: 10) encodes a VaUbi3 promoter (SEQ ID NO: 11), a chloroplast transit peptide (SEQ ID NO: 12), an SpcN (SEQ ID NO: 13), and a Nos terminator (SEQ ID NO: 14). The LacZ cassette (SEQ ID NO: 17) encodes the LacZ promoter (SEQ ID NO: 18) and LacZ gene (SEQ ID NO: 19). The bacterial selection marker cassette (SEQ ID NO: 20) encodes the KanR promoter (SEQ ID NO: 21) and KanR gene (SEQ ID NO: 22).

FIG. 5B illustrates an example plasmid vector 560 that includes a DNA sequence coding for a TALEN associated with a low THC transgene, sometimes herein referred to as the “low THC TALEN vector”. The plasmid vector 560 contains the gene encoding left-half TALEN and right half-TALEN associated with a transgene that causes reduced THC content in the transformed Cannabaceae plant or plant part, which are referred to below as the left TALEN cassette and right TALEN cassette. The plasmid vector 560 includes a YFP reporter cassette which encodes the YFP used to select for transformed plant parts. The plasmid vector 560 further includes a plant selectable marker cassette, which is in reverse orientation on the plasmid, as previously described, the features of which are not repeated. The plasmid backbone also contains a bacterial selection marker cassette that encodes the KanR gene for selection and maintenance of the plasmid within the A. rhizogenes strain, and which is in reverse orientation on the plasmid. The Cannabaceae plant part was transformed using the 18R12 strain. The plasmid vector 560 sequence is illustrated by SEQ ID NO: 23. Further identified is the sequence of the left TALEN cassette (SEQ ID NO: 24), the right TALEN cassette (SEQ ID NO: 32), the YFP reporter cassette (SEQ ID NO: 36), the plant selectable marker cassette (SEQ ID NO: 38), and the bacterial selection marker cassette (SEQ ID NO: 20). The left TALEN cassette (SEQ ID NO: 24) encodes the Nos promoter (SEQ ID NO: 25), left TAL effector N-terminus (SEQ ID NO: 26), CsTHCAS_T22-L1 binding domain (SEQ ID NO: 27), left TAL effector C-terminus (SEQ ID NO: 28), linker (SEQ ID NO: 29), Fok1 (SEQ ID NO: 30), and a Nos terminator (SEQ ID NO: 31). The right TALEN cassette (SEQ ID NO: 32) encodes the Nos promoter (SEQ ID NO: 25), right TAL effector N-terminus (SEQ ID NO: 33), CsTHCAS_T22-R1 binding domain (SEQ ID NO: 34), right TAL effector C-terminus (SEQ ID NO: 35), linker (SEQ ID NO: 29), Fok1 (SEQ ID NO: 30), and a Nos terminator (SEQ ID NO: 31). The YFP reporter cassette (SEQ ID NO: 36) encodes the FMV promoter (SEQ ID NO: 3), YFP CDS (SEQ ID NO: 37) and the Rbcs-E9 terminator (SEQ ID NO: 9). The plant selectable marker cassette (SEQ ID NO:38) encodes a 35S promoter (SEQ ID NO: 39), a ST LSI nptII intron (SEQ ID NO: 40), an NptII exon (SEQ ID NO: 41), and a 35S terminator (SEQ ID NO: 42). The bacterial selection marker cassette (SEQ ID NO: 20) encodes the KanR promoter (SEQ ID NO: 21) and KanR gene (SEQ ID NO: 22).

FIG. 5C illustrates an example plasmid vector 570 that includes a DNA sequence coding for a TALEN associated with a low THC transgene, sometimes herein referred to as the “low THC spec YFP vector”. The plasmid vector 570 contains the gene encoding left-half TALEN and right half-TALEN associated with a transgene that causes reduced THC content in the transformed Cannabaceae plant or plant part, which are referred to below as the left TALEN cassette and right TALEN cassette. The plasmid vector 570 includes a YFP reporter cassette which encodes the YFP used to select for transformed plant parts. The plasmid vector 570 further includes a plant selectable marker cassette, which is in reverse orientation on the plasmid, as previously described, the features of which are not repeated. The plasmid backbone also contains a bacterial selection marker cassette that encodes the KanR gene for selection and maintenance of the plasmid within the A. rhizogenes strain, and which is in reverse orientation on the plasmid. The Cannabaceae plant part was transformed using the 18R12 strain. The plasmid vector 570 sequence is illustrated by SEQ ID NO: 43. Further identified is the sequence of the left TALEN cassette (SEQ ID NO: 44), the right TALEN cassette (SEQ ID NO: 48), the YFP reporter cassette (SEQ ID NO: 36), the plant selectable marker cassette (SEQ ID NO: 10), and the bacterial selection marker cassette (SEQ ID NO: 20). The left TALEN cassette (SEQ ID NO: 44) encodes the VaUbi3 promoter (SEQ ID NO: 11), left TAL effector N-terminus (SEQ ID NO: 45), CsTHCAS_T22-L1 binding domain (SEQ ID NO: 46), left TAL effector C-terminus (SEQ ID NO: 47), linker (SEQ ID NO: 29), Fok1 (SEQ ID NO: 30), and a Nos terminator (SEQ ID NO: 31). The right TALEN cassette (SEQ ID NO: 48) encodes the VaUbi3 promoter (SEQ ID NO: 11), right TAL effector N-terminus (SEQ ID NO: 49), CsTHCAS_T22-R1 binding domain (SEQ ID NO: 50), right TAL effector C-terminus (SEQ ID NO: 51), linker (SEQ ID NO: 29), Fok1 (SEQ ID NO: 30), and a Nos terminator (SEQ ID NO: 31). The YFP reporter cassette (SEQ ID NO: 36) encodes the FMV promoter (SEQ ID NO: 3), YFP CDS (SEQ ID NO: 37) and the Rbcs-E9 terminator (SEQ ID NO: 9). The plant selectable marker cassette (SEQ ID NO: 10) encodes a VaUbi3 promoter (SEQ ID NO: 11), a chloroplast transit peptide (SEQ ID NO: 12), an SpcN (SEQ ID NO: 13), and a Nos terminator (SEQ ID NO: 14). The bacterial selection marker cassette (SEQ ID NO: 20) encodes the KanR promoter (SEQ ID NO: 21) and KanR gene (SEQ ID NO: 22).

FIG. 5D illustrates an example plasmid vector 572 that includes a DNA sequence coding for a TALEN associated with a phytoene desaturase (PDS) transgene. The plasmid vector 572 encodes a left-half TAL effector with a PDS binding domain fused to an SSP DnaE intein-N and a right half-TAL effector with a PDS binding domain fused to an SSP intein-N, which are referred to below as the left TALE intein cassette and right TALE intein cassette. The plasmid vector further encodes the Fok1 endonuclease fused to an SSP DnaE intein-C, which is referred to below as the endonuclease cassette. The plasmid vector 572 includes a YFP reporter cassette, which is in reverse orientation on the plasmid, as previously described. The plasmid backbone also contains a bacterial selection marker cassette that encodes the KanR gene, and which is in reverse orientation on the plasmid. The plasmid vector 572 sequence is illustrated by SEQ ID NO: 52. Further identified is the sequence of the left TALE intein cassette (SEQ ID NO: 57), the right TALE intein cassette (SEQ ID NO: 64), the YFP reporter cassette (SEQ ID NO: 53), the endonuclease cassette (SEQ ID NO: 66), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 57) encodes the VaUbi3 promoter (SEQ ID NO: 58), N-terminus (SEQ ID NO: 59), PDS left binding domain (SEQ ID NO: 60), feature 10 (SEQ ID NO: 61), intein-N(SEQ ID NO: 62), and Nos terminator (SEQ ID NO: 63). Feature 10 is a portion of the C40 domain of the yeast_1NLS_HAtag_N152_C40 sequence that does not encode the Fok1 endonuclease. For example, for the yeast_1NLS_HAtag_N152_C40 sequence containing the Repeat Variable Diresidues (RVDs,) Fok1 is removed and replaced with the inteins, e.g., Gp41-1 int-N or SSP DnaE intein-N. The N-terminus includes the N-terminal domain of yeast_1NLS_HAtag_N152_C40. The right TALE intein cassette (SEQ ID NO: 64) encodes the VaUbi3 promoter (SEQ ID NO: 58), N-terminus (SEQ ID NO: 59), PDS right binding domain (SEQ ID NO: 65), feature 10 (SEQ ID NO: 61), intein-N(SEQ ID NO: 62), and Nos terminator (SEQ ID NO: 63). The endonuclease cassette (SEQ ID NO: 66) encodes the pMtEF1A promoter (SEQ ID NO: 67), feature 10 (SEQ ID NO: 68), intein-C CDS (SEQ ID NO: 69), Fok1 (SEQ ID NO: 70), yeast_1NLS_HAtag_N152_C40 (SEQ ID NO: 71), and a Nos terminator (SEQ ID NO: 63). The yeast_1NLS_HAtag_N152_C40 is a small protein containing a nuclear localization signal (NLS) and the half TALENs are inserted between the N- and C-terminal domains (e.g., N-terminus) of the protein. The YFP reporter cassette (SEQ ID NO: 53) encodes the FMV promoter (SEQ ID NO: 54), YFP CDS (SEQ ID NO: 55), and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

FIG. 5E illustrates an example plasmid vector 574 that includes a DNA sequence coding for a TALEN associated with the PDS transgene. The plasmid vector 574 encodes a left-half TAL effector with a PDS binding domain fused to a Gp41-1 intein-N and a right half-TAL effector with a PDS binding domain fused to a Gp41-1 intein-N, which are referred to below as the left TALE intein cassette and right TALE intein cassette. The plasmid vector further encodes the Fok1 endonuclease fused to a Gp41-1 intein-C, which is referred to below as the endonuclease cassette. The plasmid vector 574 includes a YFP reporter cassette, which is in reverse orientation on the plasmid, as previously described, the features of which are not repeated. The plasmid backbone also contains a bacterial selection marker cassette, which is in reverse orientation on the plasmid. The plasmid vector 574 sequence is illustrated by SEQ ID NO: 77. Further identified is the sequence of the left TALE intein cassette (SEQ ID NO: 78), the right TALE intein cassette (SEQ ID NO: 81), the YFP reporter cassette (SEQ ID NO: 53), the endonuclease cassette (SEQ ID NO: 82), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 78) encodes the VaUbi3 promoter (SEQ ID NO: 58), N-terminus (SEQ ID NO: 59), PDS left binding domain (SEQ ID NO: 60), feature 10 (SEQ ID NO: 79), intein-N(SEQ ID NO: 80), and Nos terminator (SEQ ID NO: 63). The right TALE intein cassette (SEQ ID NO: 81) encodes the VaUbi3 promoter (SEQ ID NO: 58), N-terminus (SEQ ID NO: 59), PDS right binding domain (SEQ ID NO: 65), feature 10 (SEQ ID NO: 79), intein-N(SEQ ID NO: 80), and Nos terminator (SEQ ID NO: 63). The endonuclease cassette (SEQ ID NO: 82) encodes the pMtEF1A promoter (SEQ ID NO: 67), feature 10 (SEQ ID NO: 83), intein-C CDS (SEQ ID NO: 84), Fok1 (SEQ ID NO: 70), yeast_1NLS_HAtag_N152_C40 (SEQ ID NO: 71), and a Nos terminator (SEQ ID NO: 63). The YFP reporter cassette (SEQ ID NO: 53) encodes the FMV promoter (SEQ ID NO: 54), YFP CDS (SEQ ID NO: 55), and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

FIG. 5F illustrates an example plasmid vector 576 that includes a DNA sequence coding for a TALEN associated with a THCAS transgene. The plasmid vector 576 encodes a left-half TAL effector with a THCAS binding domain fused to an SSP DnaE intein-N and a right half-TAL effector with a THCAS binding domain fused to an SSP intein-N, which are referred to below as the left TALE intein cassette and right TALE intein cassette. The plasmid vector further encodes the Fok1 endonuclease fused to an SSP DnaE intein-C, which is referred to below as the endonuclease cassette. The plasmid vector 576 includes a YFP reporter cassette, which is in reverse orientation on the plasmid, as previously described, the features of which are not repeated. The plasmid backbone also contains a bacterial selection marker cassette, which is in reverse orientation on the plasmid. The plasmid vector 576 sequence is illustrated by SEQ ID NO: 85. Further identified is the sequence of the left TALE intein cassette (SEQ ID NO: 86), the right TALE intein cassette (SEQ ID NO: 90), the YFP reporter cassette (SEQ ID NO: 53), the endonuclease cassette (SEQ ID NO: 66), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 86) encodes the VaUbi3 promoter (SEQ ID NO: 58), N-terminus (SEQ ID NO: 59), THCAS left binding domain (SEQ ID NO: 87), feature 10 (SEQ ID NO: 88), intein-N(SEQ ID NO: 89), and Nos terminator (SEQ ID NO: 63). The right TALE intein cassette (SEQ ID NO: 90) encodes the VaUbi3 promoter (SEQ ID NO: 58), N-terminus (SEQ ID NO: 59), THCAS right binding domain (SEQ ID NO: 91), feature 10 (SEQ ID NO: 88), intein-N(SEQ ID NO: 89), and Nos terminator (SEQ ID NO: 63). The endonuclease cassette (SEQ ID NO: 66) encodes the pMtEF1A promoter (SEQ ID NO: 67), feature 10 (SEQ ID NO: 68), intein-C CDS (SEQ ID NO: 69), Fok1 (SEQ ID NO: 70), yeast_1NLS_HAtag_N152_C40 (SEQ ID NO: 71), and a Nos terminator (SEQ ID NO: 63). The YFP reporter cassette (SEQ ID NO: 53) encodes the FMV promoter (SEQ ID NO: 54), YFP CDS (SEQ ID NO: 55) and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

FIG. 5G illustrates an example plasmid vector 578 that includes a DNA sequence coding for a TALEN associated with the THCAS transgene. The plasmid vector 578 encodes a left-half TAL effector with a THCAS binding domain fused to a Gp41-1 intein-N and a right half-TAL effector with a THCAS binding domain fused to an Gp41-1 intein-N, which are referred to below as the left TALE intein cassette and right TALE intein cassette. The plasmid vector further encodes the Fok1 endonuclease fused to an Gp41-1 intein-C, which is referred to below as the endonuclease cassette. The plasmid vector 576 includes a YFP reporter cassette, which is in reverse orientation on the plasmid, as previously described, the features of which are not repeated. The plasmid backbone also contains a bacterial selection marker cassette, which is in reverse orientation on the plasmid. The plasmid vector 576 sequence is illustrated by SEQ ID NO: 92. Further identified is the sequence of the left TALE intein cassette (SEQ ID NO: 93), the right TALE intein cassette (SEQ ID NO: 97), the YFP reporter cassette (SEQ ID NO: 53), the endonuclease cassette (SEQ ID NO: 82), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 93) encodes the VaUbi3 promoter (SEQ ID NO: 58), N-terminus (SEQ ID NO: 59), THCAS left binding domain (SEQ ID NO: 94), feature 10 (SEQ ID NO: 95), intein-N(SEQ ID NO: 96), and Nos terminator (SEQ ID NO: 63). The right TALE intein cassette (SEQ ID NO: 97) encodes the VaUbi3 promoter (SEQ ID NO: 58), N-terminus (SEQ ID NO: 59), THCAS right binding domain (SEQ ID NO: 98), feature 10 (SEQ ID NO: 95), intein-N(SEQ ID NO: 96), and Nos terminator (SEQ ID NO: 63). The endonuclease cassette (SEQ ID NO: 82) encodes the pMtEF1A promoter (SEQ ID NO: 67), feature 10 (SEQ ID NO: 83), Intein-C CDS (SEQ ID NO: 84), Fok1 (SEQ ID NO: 70), yeast_1NLS_HAtag_N152_C40 (SEQ ID NO: 71), and a Nos terminator (SEQ ID NO: 63). The YFP reporter cassette (SEQ ID NO: 53) encodes the FMV promoter (SEQ ID NO: 54), YFP CDS (SEQ ID NO: 55) and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

FIG. 5H illustrates an example plasmid vector 580 that includes a DNA sequence coding for a TALEN associated with the PDS transgene. The plasmid vector 580 encodes a left-half TAL effector with PDS binding domain fused to a Gp41 intein-N and a right half-TAL effector with a PDS binding domain fused to an Gp41-1 intein-N, which are referred to below as the left TALE intein cassette and right TALE intein cassette. The plasmid vector further encodes the Fok1 endonuclease fused to a Gp41-1 intein-C, which is referred to below as the endonuclease cassette. The plasmid vector 580 includes a YFP reporter cassette, which is in reverse orientation on the plasmid, as previously described, the features of which are not repeated. The plasmid backbone also contains a bacterial selection marker cassette, which is in reverse orientation on the plasmid. The plasmid vector 580 sequence is illustrated by SEQ ID NO: 99. Further identified is the sequence of the left TALE intein cassette (SEQ ID NO: 100), the right TALE intein cassette (SEQ ID NO: 104), the YFP reporter cassette (SEQ ID NO: 53), the endonuclease cassette (SEQ ID NO: 106), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 100) encodes the VaUbi3 promoter (SEQ ID NO: 58), N-terminus (SEQ ID NO: 59), PDS left binding domain (SEQ ID NO: 101), feature 10 (SEQ ID NO: 102), intein-N(SEQ ID NO: 103), and Nos terminator (SEQ ID NO: 63). The right TALE intein cassette (SEQ ID NO: 104) encodes the VaUbi3 promoter (SEQ ID NO: 58), N-terminus (SEQ ID NO: 59), PDS right binding domain (SEQ ID NO: 105), feature 10 (SEQ ID NO: 102), intein-N(SEQ ID NO: 103), and Nos terminator (SEQ ID NO: 63). The endonuclease cassette (SEQ ID NO: 106) encodes the pMtEF1A promoter (SEQ ID NO: 67), feature 10 (SEQ ID NO: 107), intein-C CDS (SEQ ID NO: 84), Fok1 (SEQ ID NO: 70), yeast_1NLS_HAtag_N152_C40 (SEQ ID NO: 71), and a Nos terminator (SEQ ID NO: 63). The YFP reporter cassette (SEQ ID NO: 53) encodes the FMV promoter (SEQ ID NO: 54), YFP CDS (SEQ ID NO: 55) and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

FIG. 5I illustrates an example plasmid vector 582 that includes a DNA sequence coding for a TALEN associated with the THCAS transgene. The plasmid vector 582 contains the gene encoding left-half TAL effector with THCAS binding domain fused to a Gp41-1 intein-N and a right half-TAL effector with a THCAS binding domain fused to an Gp41-1 intein-N, which are referred to below as the left TALE intein cassette and right TALE intein cassette. The plasmid vector further encodes the Fok1 endonuclease fused to a Gp41-1 intein-C, which is referred to below as the endonuclease cassette. The plasmid vector 582 includes a YFP reporter cassette, which is in reverse orientation on the plasmid, as previously described, the features of which are not repeated. The plasmid backbone also contains a bacterial selection marker cassette, which is in reverse orientation on the plasmid. The plasmid vector 582 sequence is illustrated by SEQ ID NO: 108. Further identified is the sequence of the left TALE intein cassette (SEQ ID NO: 109), the right TALE intein cassette (SEQ ID NO: 112), the YFP reporter cassette (SEQ ID NO: 53), the endonuclease cassette (SEQ ID NO: 114), the bacterial selection marker cassette (SEQ ID NO: 74), the right T-DNA border (SEQ ID NO: 72), and the left T-DNA border (SEQ ID NO: 73). The left TALE intein cassette (SEQ ID NO: 109) encodes the VaUbi3 promoter (SEQ ID NO: 58), N-terminus (SEQ ID NO: 59), THCAS left binding domain (SEQ ID NO: 110), feature 10 (SEQ ID NO: 111), intein-N(SEQ ID NO: 103), and Nos terminator (SEQ ID NO: 63). The right TALE intein cassette (SEQ ID NO: 112) encodes the VaUbi3 promoter (SEQ ID NO: 58), N-terminus (SEQ ID NO: 59), THCAS right binding domain (SEQ ID NO: 113), feature 10 (SEQ ID NO: 111), intein-N(SEQ ID NO: 103), and Nos terminator (SEQ ID NO: 63). The endonuclease cassette (SEQ ID NO: 114) encodes the pMtEF1A promoter (SEQ ID NO: 67), feature 10 (SEQ ID NO: 115), intein-C CDS (SEQ ID NO: 84), Fok1 (SEQ ID NO: 70), yeast_1NLS_HAtag_N152_C40 (SEQ ID NO: 71), and a Nos terminator (SEQ ID NO: 63). The YFP reporter cassette (SEQ ID NO: 53) encodes the FMV promoter (SEQ ID NO: 54), YFP CDS (SEQ ID NO: 55) and the Rbcs-E9 terminator (SEQ ID NO: 56). The bacterial selection marker cassette (SEQ ID NO: 74) encodes the KanR promoter (SEQ ID NO: 75) and KanR gene (SEQ ID NO: 76).

Some embodiments were directed to preparing a starter culture of a bacterium strain and preparing the bacterial culture medium including the bacterium strain. The bacterium stain included A. rhizogenes bacterium strain (18r12). The A. rhizogenes strain was streaked onto a plate on AB +Kan50 medium. The single colony was inoculated into a 15 mL YEP culture plus 7.5 uL Kan50, which were all in a 50 mL vented conical tube. In some experiments, a second culture is inoculated with another single colony as a backup. The culture was placed at an angle in a 28° C. shaker (220 rpm) for around eight hours. The OD_{STARTER} was measured to ideally be at an optical density (OD) 600 between 0.2 and 0.4. 250 mL flasks were prepared with 49 mL liquid AB minimal culture media, 1 mL YEP starter culture, and 25 uL Kan50, and grown for 20 hours at 28° C. on the shaker (220 rpm). As previously described, the bacterium strain can be transformed to carry the heterologous nucleotide sequence.

The Cannabaceae seeds were prepped by sterilizing and imbibing the seed. The following steps were performed in a fume hood: 1) a 50 mL tube containing around 100 seeds was taken and serological pipette was used to add 10 mL of 12M sulfuric acid; 2) the 50 mL tube was closed, and gently shaken for 10 seconds; 3) the 50 mL tube was opened and a serological pipette was used to remove the sulfuric acid; 4) the sulfuric acid waste was pipetted into a glass waste beaker containing 400 mL of ddH₂O; 5) to rinse, 45 ml of sterile ddH₂O was added to the 50 mL tube containing the seeds; 6) the 50 ml tube was closed and gently shaken for 10 seconds; 7) a serological pipette was used to remove the ddH₂O from the 50 ml tube; and 8) to rinse, 45 mL of fresh ddH₂O was added to the 50 mL tube, the tube was closed, and the tube was brought to a laminar flow hood. The ddH₂O was removed from the 50 mL tube and 45 mL of 30% H₂O₂ was added. The 50 ml tube was closed and placed on a rotary shaker at 20 rpm for 10-20 minutes. After 10-20 minutes, the 50 mL tube was removed from the rotary shaker and brought back to the laminar flow hood. A serological pipette was used to remove the H₂O₂ from the 50 mL tube. A ddH₂O rinse was performed five times (5×) on the 50 mL tube containing the seeds. After the fifth ddH₂O rinse, the seeds, which are sterilized, were poured out onto a plate containing solid MS medium and 8 mL of 2% v/v PPM was added directly to the plate and placed on a shaker set to 70 rpm overnight. This was used to avoid over imbibition and the PPM eliminates endophytic fungal contamination.

The starter culture of the bacterium strain, the seed imbibition occurred on day 1, in some experimental embodiments.

The starter culture was prepared for infection. OD, bottle, and centrifuge three 50 mL flask of starter culture at 5400 rpm for 10 minute. The supernatant was removed and 150 mL of TDZ infection medium was added to resuspend the pellet. 75 μL of 40 mg/mL Acetosyringone was added to give final [Acetosyringone]=100 uM to prepare the infection medium. 0.02% v/v Silwet L-77 was added to the resuspended bacterium in the infection medium.

The imbibed seeds were used to extract the Cannabaceae meristematic region and/or EA. For example, 20 mL of TDZ infection medium was added to 100×25 mm petri plates to form infection plates, and the imbibed seeds remained on the plate with the solid MS medium with 2% v/v PPM. A sufficient number of infection plates were used to provide around 75 Cannabaceae meristematic regions and/or EAs per plate and one infection plate per plate of imbibed seeds. Sterile forceps were used to gently remove cotyledons, primary leaves, and the seed coat from each EA. Once the seed coats, cotyledons, primary leaves were removed, the EAs were placed in the petri plates with the 20 mL TDZ infection medium prior to inoculation with the bacterium, such as 18r12.

The bacterium strain that is cultured and suspended, such as in a TDZ infection medium, is sonicated and inoculated with the imbibed EAs and/or meristematic regions. For example, the TDZ infection medium was pipetted off from the infection plates and 10 mL of the resuspended bacterium was added. The infection plates were parafilmed and sonicated one plate at a time for 80 seconds. After the sonication, 20 mL of fresh bacterium was added. The plates were then incubated for 30 minutes at room temperature in a laminar flow hood.

After sonicating and inoculating, the meristematic region and/or EAs were co-cultivated with the bacterium strain. For example, the remaining bacterium was pipetted off the infection plates. The meristematic regions and/or EAs were then transferred to a new 100×15 mm petri dish containing a piece of sterile filter paper wetted with 750 μL of sterile ddH₂O, sometimes herein referred to as the “co-cultivation plate”. This assisted in drying off excess bacterium. The EAs from the infection plate (e.g., all around 75) were gathered into a mound using bent jaw forceps and the mound of EAs were transferred to a prepared co-cultivation plate which has the sterile filter paper wetted with sterile ddH₂O, with the EAs of mound still mounded together. The co-cultivation plate was wrapped with a layer of parafilm and incubated for 2-4 days in 24 hr ambient light (0 umol/m⁻²/s⁻²) at 23° C., 40% humidity.

In other experiments, a co-cultivation medium was used. For example, the single meristematic regions and/or EAs were transferred onto the co-cultivation plates having co-cultivation medium thereon by gently picking up one meristematic region and/or EA at a time and plating around 10 EAs in a spread out fashion one each co-cultivation plate. The co-cultivation plates were wrapped in layer of parafilm and incubated for two to four days in 16/8 hour ambient light (30 umol/m⁻²/s⁻¹) at 23° C., 40% humidity.

The preparation of the infection medium, the extraction of the Cannabaceae meristematic region and/or EA, the sonication and inoculation, and the co-cultivation occurred or at least started on day 2, in some experimental embodiments.

After the co-cultivation, the meristematic regions and/or EAs were transferred to a Cannabaceae liquid (CL) medium as a recovery step to help reduce or eliminate contamination, such as CL media disclosed below. For example, the meristematic region and/or EA were transferred to CL media in a 100×25 mm petri dish and sealed with parafilm. In some experiments, around 50-100 EAs were plated per plate for 3 days in 16/8 hour light of 50-100 umol/m⁻²/s⁻¹ at 23° C., 40% humidity in a Conviron incubator.

After the recovery step, the meristematic regions and/or EAs are transferred to a SIM containing TDZ in a range of 2-10 mg/L. The EAs were oriented with the radicle down into and/or the meristematic region were oriented facing up in the SIM and apical meristem up. In some embodiments, the EAs were submerged in solid SIM. The bottom of a second sterile 100×25 mm plate was used as a lid and sealed with micropore tape. In some embodiments, the EAs were cultured 10 EAs per plate for 18 days under 100 umol/m²/sec white fluorescent light at room 23+/−1° C.

After shoot induction, a first shoot elongation was performed. For example, the meristematic regions and/or EAs were transferred to a first SEM, such as to SEM I+S100 media. The radicles were cut and the EAs were oriented with the radicle down into the first SEM media and apical meristem up. The bottom of a second sterile 100×25 mm plate was used as a lid and sealed with micropore tape. In some embodiments, the EAs were cultured 5 EAs per plate for 21 days under 100 umol/m²/sec white fluorescent light at 23+/−1° C.

After the first shoot elongation, a second shoot elongation was performed. For example, the meristematic regions and/or EAs were then transferred to a second SEM, such as to SEM I+S150 media. The EAs were oriented with the radicle down into the second SEM media and apical meristem up. The bottom of a second sterile 100×25 mm plate was used as a lid and sealed with micropore tape. In some embodiments, the EAs were cultured 5 EAs per plate for 21 days under 100 umol/m²/sec white fluorescent light at 23+/−1° C.

After the second shoot elongation, the elongated shoots were rooted. Positive looking shoots (preferably 2″ in height, minimum of 1″ tall shoots with non-bleached leaves) were cut and rooted on a Cannabaceae RM DKW+0.5 IBA media in phytatrays under LEDs for around 14 days until the shoots have developed at least two primary roots that are at least one cm in length. The shoots were subcultured onto fresh RM around every 7-14 days until new primary roots and root hair structures developed and before sending to acclimation in soil.

To create AB Salts (20×), the following protocol and volumes were used:
700 mL of ddH₂O; 20 g of NH₄Cl; 6 g of MgSO₄*7H₂O; 3 g of KCl; 0.2 g of CaCl₂); 50 mg of FeSO₄*7H₂O; the pH was adjusted to 7.0 with KOH; and the solution was brought to volume with 1000 mL of ddH₂O.

To create AB Buffer (20×), the following protocol and volumes were used to form 1 L of the media:
700 mL of ddH₂O; 60 g of K₂HPO₄; 20 g of NaH₂PO₄; and the solution was brought to volume with 1000 mL of ddH₂O.

To create AB minimal agar media (solid), the following protocol and volumes were used to form 1 L of media:
700 mL of ddH₂O; 5 g of Sucrose; 15 g of molecular grade agar; the solution was brought to volume with 900 mL of ddH₂O; 50 mL of 20×AB Salts; and 50 mL of 20×AB Buffer.
   The media was autoclaved on liquid cycle for 25 minutes. To create the AB +Kan50 medium, 50 mg/L of Kanamycin was added to the AB medium.

To create AB minimal media (liquid), the following protocol and volumes were used to form 1 L of media:
700 mL of ddH₂O; 5 g of Sucrose; the solution was brought to volume with 900 mL of ddH₂O; 50 mL of 20×AB Salts; and 50 mL of 20×AB Buffer.
   The media was autoclaved on liquid cycle for 25 minutes and cooled to 55° C. and poured into 100×15 mm plates.

To create YEP media (liquid), the following protocol and volumes were used to form 1 L of media:
800 mL of ddH₂O; 10 g of Bacto-peptone; 5 g of Yeast extract; 5 g of NaCl; and the solution was brought to volume with 1000 mL of ddH₂O. The media was filter sterilized.

To create TDZ infection media, the following protocol and volumes were used to form 1 L of media:
800 mL of ddH₂O; 1.305 g DKW Basal Salts (D190); 25 mL 20×AB Salts; 25 mL 20×AB Buffer; 1 g Potassium Nitrate; 20 g Glucose; 5 g MES (M825); 0.1 mL Gamborg's B5 Vitamins (G219) [1000×]; the solution was brought to 1000 mL volume with ddH₂O; the pH was adjusted to 5.4 with titration of KOH/HCl; 1.0 mL TDZ (T8118) [1 mg/mL].
   The media was filter sterilized and thiols were added the day of use. The thiols added included 2.0 mL Dithiothreitol [77 mg/ml], 4.96 mL Sodium Thiosulfate·5H2O [50 mg/ml], and 8 mL L-Cysteine [50 mg/ml]. In some embodiments, 0.02% v/v Silwet L-77 was added the day of use.

To create CL media, the following protocol and volumes were used to form 1 L of media:
800 mL of ddH₂O; 30 g Sucrose (S391); 5.22 g DKW Basal Salts (D190); 1 g MES (M825); 1 mL PPM; 1 mL Gamborg's B5 Vitamins (G219) [1000×]; the solution was brought to volume with 168.9 mL ddH₂O; and the pH was adjusted to 5.7 with titration of KOH.
   The media was filter sterilized and the following were added post autoclave: 2 mL TDZ (T8119) [1 mg/mL]; 2.0 mL Asparagine (A107) [25 mg/ml], 2.0 mL Glutamine (G229) [25 mg/ml], 1.0 mL Timentin (T104) [300 mg/ml], 1.2 mL Cefotaxime (C380) [250 mg/ml], and 2 mL Carbenicillin (C346) [250 mg/mL].

To create the co-cultivation media, such as co-cult G DKW, the following protocol and volumes were used to form 1 L of media:
800 mL of ddH₂O; 20 g Glucose; 5.22 g G DKW Basal Salts (D190); 1 g MES (M825); the solution was brought to volume with 183.75 mL ddH₂O; the pH was adjusted to 5.8 with titration of KOH; and 6 g Agar, Plant TC (A296).
   The media was autoclaved on AGAR cycle with the MediaClave and the following were added post autoclave: 0.125 mL IAA (1364) [1 mg/ml], 2 mL trans-Zeatin Riboside (Z899) [1 mg/ml], and 2 mL Acetosyringone (A1104) [20 mg/ml=100 mM]. As used herein, the G in DKW refers to gelzan (e.g., a solidifying agent) and DKW is a type of salt. However, in various experiments, the co-cultivation was performed using a piece of sterile filter paper wetted with sterile ddH₂O, and without the use of co-cultivation media.

To create SIM+S10 (TDZ2) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219) [1000×]; 8 mL Iron Chelate (F318); the solution was brought to volume with 163.532 mL ddH₂O; the pH was adjusted to 5.7 with titration of KOH; and 7 g Agar, Plant TC (A296).
   The media was autoclaved on AGAR cycle with the MediaClave and the following were added post autoclave: 2 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 0.2 mL Spectinomycin (S4014) [50 mg/ml].

To create SIM+S10 (TDZ10) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 155.532 mL ddH₂O; the pH was adjusted to 5.7 with titration of KOH; and 7 g Agar, Plant TC (A296).
   The media was autoclaved on AGAR cycle with the MediaClave and the following were added post autoclave: 10 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 0.2 mL Spectinomycin (S4014) [50 mg/ml].

To create SIM+S50 (TDZ2) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 163.532 mL ddH₂O; the pH was adjusted to 5.7 with titration of KOH; and 7 g Agar, Plant TC (A296)
   The media was autoclaved on AGAR cycle with the MediaClave and the following were added post autoclave: 2 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 1 mL Spectinomycin (S4014) [50 mg/ml].

To create SIM+S50 (TDZ10) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 155.532 mL ddH₂O; the pH was adjusted to 5.7 with titration of KOH; and 7 g Agar, Plant TC (A296).
   The media was autoclaved on AGAR cycle with the MediaClave and the following were added post autoclave: 10 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 1 mL Spectinomycin (S4014) [50 mg/mL].

To create SIM+S100 (TDZ2) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 163.532 mL ddH₂O; the pH was adjusted to 5.7 with titration of KOH; and 7 g Agar, Plant TC (A296)
   The media was autoclaved on AGAR cycle with the MediaClave and the following were added post autoclave: 2 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 2 mL Spectinomycin (S4014) [50 mg/ml].

To create SIM+S100 (TDZ10) media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 155.532 mL ddH₂O; the pH was adjusted to 5.7 with titration of KOH; and 7 g Agar, Plant TC (A296).
   The media was autoclaved on AGAR cycle with the MediaClave and the following were added post autoclave: 10 mL TDZ (T8118) [1 mg/mL], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 2 mL Spectinomycin (S4014) [50 mg/ml].

To create SEM I+S50 media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 154.594 mL ddH₂O; the pH was adjusted to 5.7 with titration of KOH; and 7 g Agar, Plant TC (A296)
   The media was autoclaved on AGAR cycle with the MediaClave and the following were added post autoclave: 0.0385 mL Gibberellic Acid (G362) [13 mg/ml], 0.1 mL IAA (1364) [1 mg/ml], 10 mL L-ascorbic acid [10 mg/ml], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 1 mL Spectinomycin (S4014) [50 mg/ml].

To create SEM I+S100 media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume with 154.594 mL ddH₂O; the pH was adjusted to 5.7 with titration of KOH; and 7 g Agar, Plant TC (A296)
   The media was autoclaved on AGAR cycle with the MediaClave and the following were added post autoclave: 0.0385 mL Gibberellic Acid (G362) [13 mg/ml], 0.1 mL IAA (1364) [1 mg/ml], 10 mL L-ascorbic acid [10 mg/ml], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 2 mL Spectinomycin (S4014) [50 mg/ml].

To create SEM I+S150 media, the following protocol and volumes were used to form 1 L of media:
800 mL of ddH₂O; 30 g Sucrose (S391); 5.22 g DKW Basal Salts (D190); 1 g MES (M825); 1 mL Gamborg's B5 Vitamins (G219); 8 mL Iron Chelate (F318); the solution was brought to volume 154.594 mL ddH₂O; the pH was adjusted to 5.7 with titration of KOH; and 7 g Agar, Plant TC (A296)
   The media was autoclaved on AGAR cycle with the MediaClave and the following were added post autoclave: 0.0385 mL Gibberellic Acid (G362) [13 mg/ml], 0.1 mL IAA (1364) [1 mg/ml], 10 mL L-ascorbic acid [10 mg/ml], 2 mL Asparagine (A107) [25 mg/ml], 2 mL Glutamine (G229) [25 mg/ml], 1.2 mL Carbenicillin (C346) [250 mg/ml], 1.2 mL Cefotaxime (C380), and 3 mL Spectinomycin (S4014 [50 mg/ml]).

To create the Cannabaceae RM DKW+0.5 IBA media, the following protocol and volumes were used to form 1 L of media:
800 mL of ddH₂O; 30 g Sucrose (S391); 5.22 g DKW Basal Salts (D190); 1 mL Gamborg's B5 Vitamins (G219); 5 mL Plant Preservation Mixture; the solution was brought to volume 174.63 mL ddH₂O; the pH was adjusted to 5.8 with titration of KOH; and 7 g Agar, Plant TC (A296)
   The media was autoclaved on AGAR cycle with the MediaClave and 0.5 mL of IBA [1 mg/1 mL) was added.

The infection medium (e.g., EA TDZ infection media), co-cultivation medium, regeneration medium (e.g., CL media,), SIM (e.g., SIM+S10 (TDZ2) media, SIM+S10 (TDZ10) media, SIM+S50 (TDZ2) media, SIM+S50 (TDZ10) media, SIM+S100 (TDZ2) media, and SIM+S100 (TDZ10) media), SEM (e.g., SEM I+S50 media, SEM I+S100 media, and SEM I+S150 media), and RM DKW described above generally comprise water, a basal salt mixture, a sugar, and one or more other components such as vitamins, selection agents, amino acids, and phytohormones. The SIM and SEMs can include nutritional sources of nitrogen, phosphorus, potassium, sulfur, calcium, magnesium, iron, boron, molybdenum, manganese, cobalt, zinc, copper, chlorine, and iodine. In some experimental embodiments, the following was performed in accordance with Table 1:

TABLE 1

| Cannabis | # Mutant | # Total | Editing |
|---|---|---|---|
| Sample | Reads | Reads | Frequency |
| 1 | 1060 | 20219 | 5.24% |
| 2 | 1125 | 17596 | 6.39% |
| 3 | 466 | 16607 | 2.81% |
| 4 | 481 | 20807 | 2.31% |
| 5 | 416 | 17736 | 2.35% |
| 6 | 586 | 11003 | 5.33% |
| 7 | 597 | 11309 | 5.28% |
| 8 | 483 | 17858 | 2.70% |
| 9 | 10316 | 49122 | 21% |
| 10 | 18306 | 59050 | 31% |

In such experimental embodiments, cannabis EAs were transformed with plasmid vector 570 as illustrated by FIG. 5C. The cannabis EAs were transformed using the Cannabaceae transformation protocol and using bacterium containing a binary vector. The binary vector contains different elements including spectinomycin selection marker, YFP visual selection marker, and a TALEN pair that is designed to knock out portions of the THCAS genes. The cannabis EAs with the 21% and 31% editing frequency were plated onto SIM+S10 (TDZ10) for three weeks.

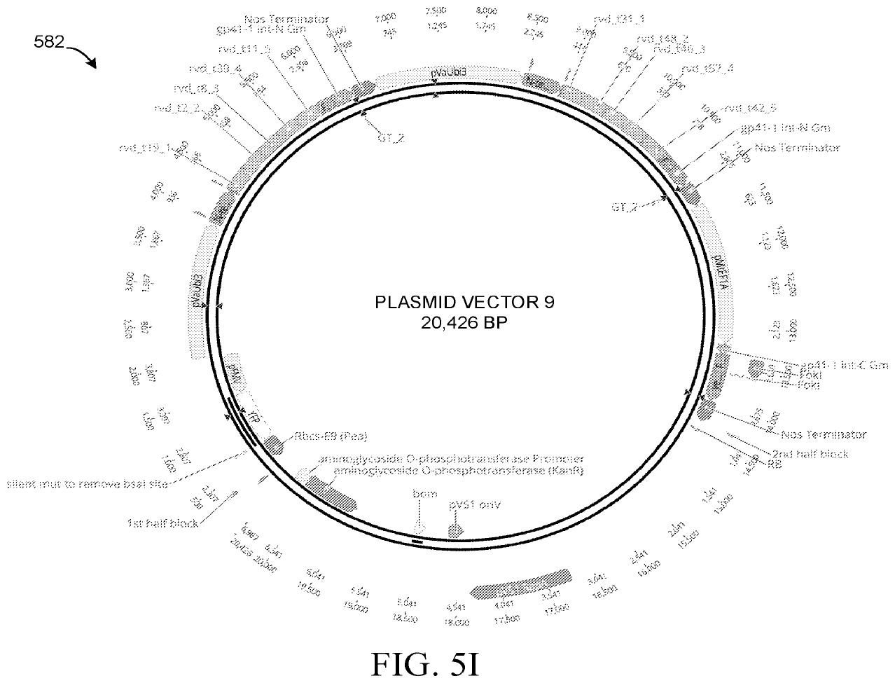

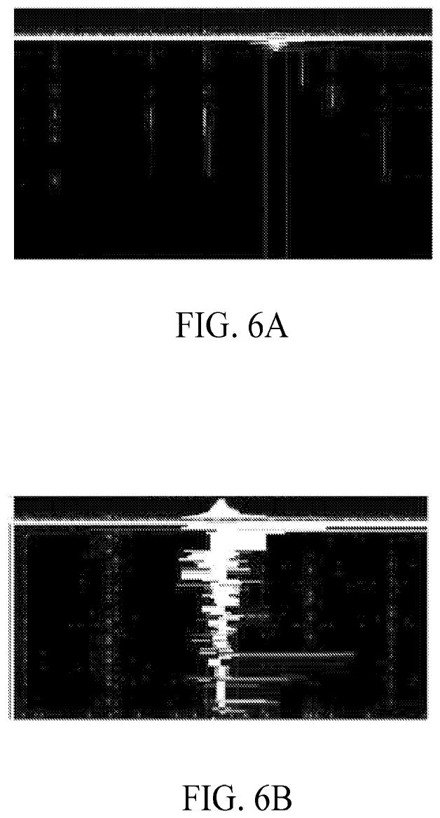

FIGS. 6A-6B include example images from samples from Table 1, consistent with the present disclosure. The images from FIG. 6A-6B are Illumina alignment images from sample 2. The white regions within the vertical yellow dotted lines show successful editing of the targeted THCAS region. FIG. 6B is a zoomed-in version of FIG. 6A that shows the reads that represent knock-out editing of the THCAS region.

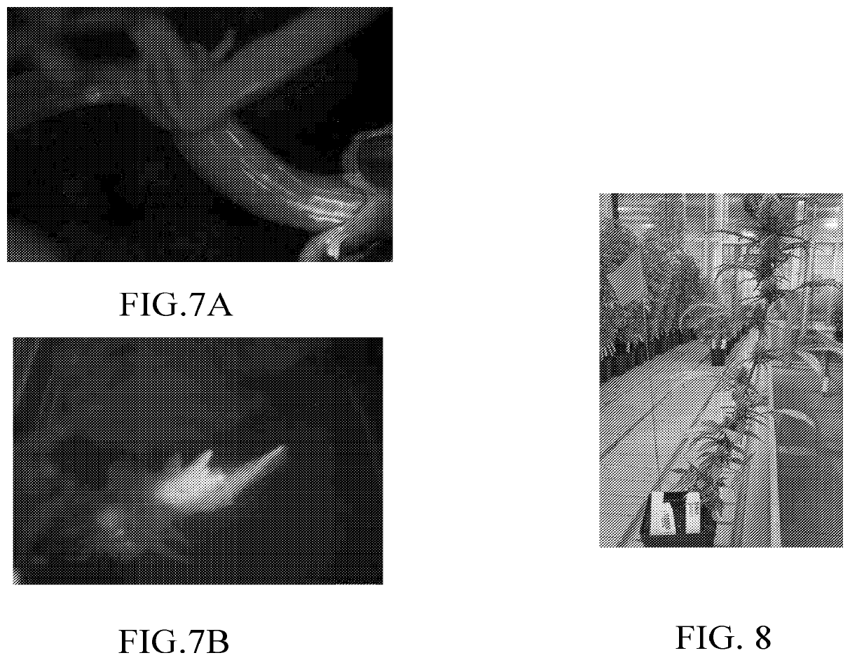

FIGS. 7A-7B are images of a cannabis seedling explant transformed with a bacterium strain, consistent with the present disclosure. In some experiments, cannabis seedling explants, with the cotyledons intact during transformation, were transformed with plasmid vector 560 as illustrated by FIG. 5B. In such experiments, the cannabis seedlings with their cotyledons intact were transformed using the Cannabaceae transformation protocol and using bacterium containing a binary vector. The binary vector contains different elements including kanamycin selection marker, YFP visual selection marker, and a TALEN pair that is designed to knock-out portions of the THCAS genes. As shown by the image of FIG. 7A, YFP is seen in the transformed explant in the stem of the explant, indicating that the transgene has been stably integrated into the cannabis genome. As shown by the image of FIG. 7B, the YFP fluorescence can also be seen in the root region of the explant.

FIG. 8 is an image of a plant regenerated from the cannabis seedling explant transformed with the bacterium as shown by FIGS. 7A-7B, consistent with the present disclosure. The plant was tissue sampled and analyzed using PCR. The PCR data shows that the plant contains the transgene delivered by the transformation method.

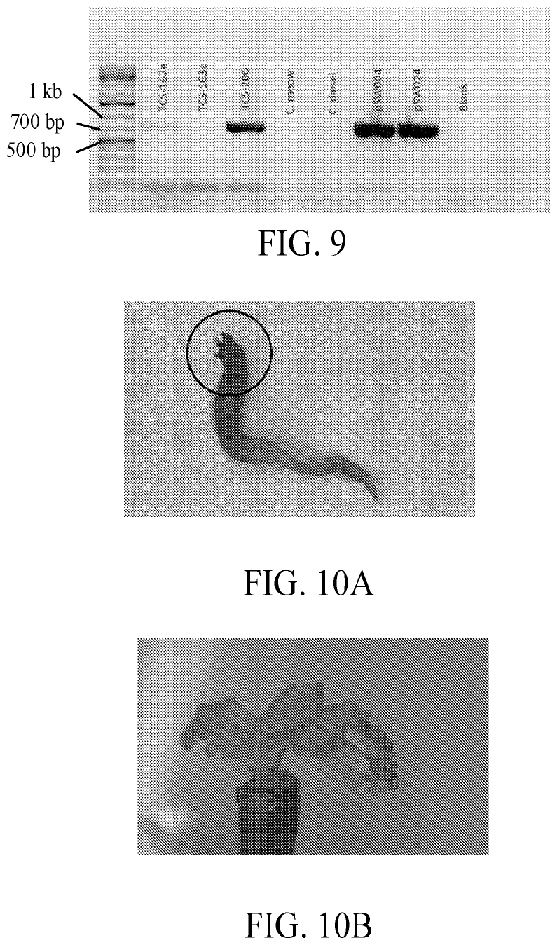

FIG. 9 is an image of PCR data from the regenerated plant shown in FIG. 8, consistent with the present disclosure. As shown, the plant has a positive band for the transgene and the band aligns well with the positive control DNA.

FIGS. 10A-10B are images of a cannabis meristematic region transformed with a bacterium strain, consistent with the present disclosure. In some embodiments, cannabis explants were transformed with plasmid vector 550 as illustrated by FIG. 5A. In the experiments, the cannabis explants that include or are implemented as a meristematic region were transformed using the transformation protocol, as described above, and using an Agrobacterium strain containing a binary vector. The binary vector contained different elements including spectinomycin selection marker and a betalain visual selection marker. The cannabis explant shows a highly efficient transformation, with the entire explant expressing the betalain color. Additionally, the explant derived from the cannabis explant evidences that the transgene successfully integrated into the regenerating tissues. Betalain is shown as being expressed in the meristem, petiole, and leaf tissue, in addition to the high levels of betalain expression in the original explant. FIG. 10A is an image of the transformed cannabis explant that shows expression of betalain and illustrates integration of the T-DNA vector. More particularly, the image of FIG. 10A illustrates a transformation of a meristematic region. FIG. 10B illustrates a plant regenerated from the explant which shows betalain expression in the meristem, petiole, and leaf tissues. The original explant shows highly efficient integration of the T-DNA vector. Furthermore, the regenerating plant derived from this explant shows betalain expression in the meristem, petiole, and leaf tissues.

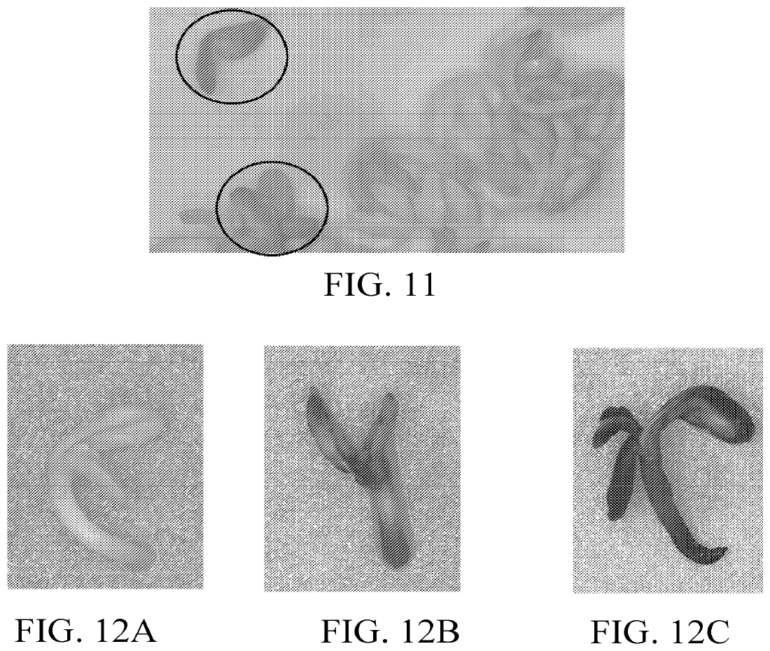

FIG. 11 is an image of a cannabis seedling transiently transformed with a bacterium strain, consistent with the present disclosure. In some experiments, transient expression of seedlings transformed with plasmid vector 550 was performed. In such embodiments, the cannabis seedlings were transformed using the transformation protocol as described above and using an Agrobacterium strain containing a binary vector. The binary vector contained different elements including spectinomycin selection marker and a betalain visual selection marker. The seedlings showed efficient transformation, with some explants transiently expressing the betalain color 45 minutes after transformation. The image of FIG. 11 illustrates the cannabis seedling showing transient expression of the betalain color 45 minutes after transformation. The circled explants are transiently expressing betalain, while the remaining are not.

FIGS. 12A-12C are images of a cannabis seedling explant stably transformed with a bacterium strain, consistent with the present disclosure. In some embodiments, cannabis seedling explants were stably transformed with plasmid vector 550. The seedlings showed efficient transformation, with example explants showing stable transformation of the RUBY vector compared to the control. FIG. 12A illustrates an image of a control explant that is not transformed with the RUBY vector. FIGS. 12B and 12C show stably transformed cannabis seedling explants. As shown by FIGS. 12B-12C, the betalain expression can be seen in the explant.

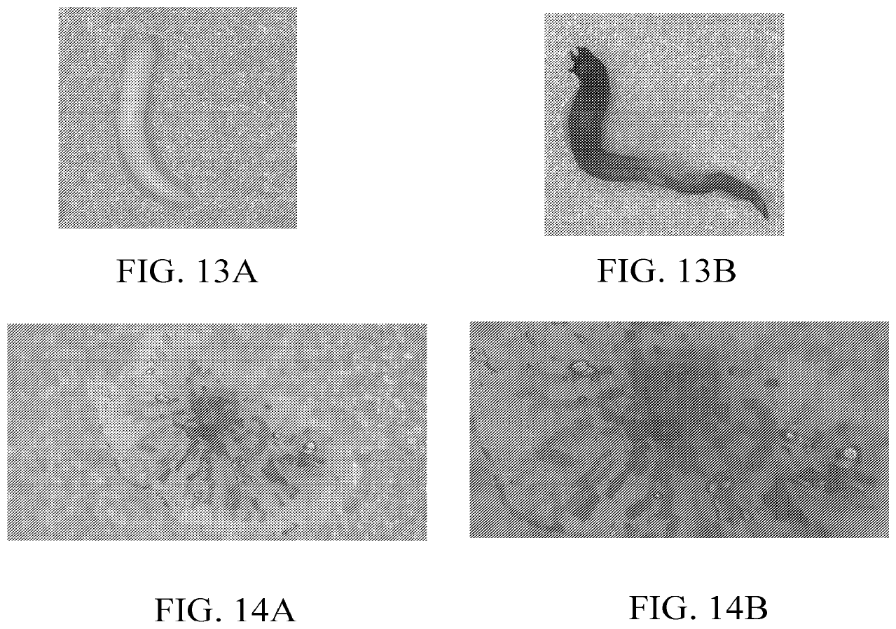

FIGS. 13A-13B are images of a cannabis EA stably transformed with a bacterium strain, consistent with the present disclosure. In some embodiments, cannabis EAs were transformed with a plasmid vector 550. The EAs showed stable expression of the transgene, compared to no betalain expression in the wild-type EA control. FIG. 13A illustrates an image of a control explant that is not transformed with the RUBY vector. FIG. 13B shows a stably transformed cannabis seedling explant. As shown by FIG. 13B, the betalain expression can be seen in the explant. The transformed cannabis EA shows stable expression of betalain throughout the entire explant.

FIGS. 14A-14B are images of a cannabis seedling explant stably transformed with a bacterium strain, consistent with the present disclosure. In some embodiments, cannabis seedling explants were transformed with a plasmid vector 550. The seedling explants were thinly sliced and imaged using a microscope. The sections show cells that are expressing betalain. FIGS. 14A-14B illustrate cross sections of a cannabis seedling that has been stably transformed with the transgene.

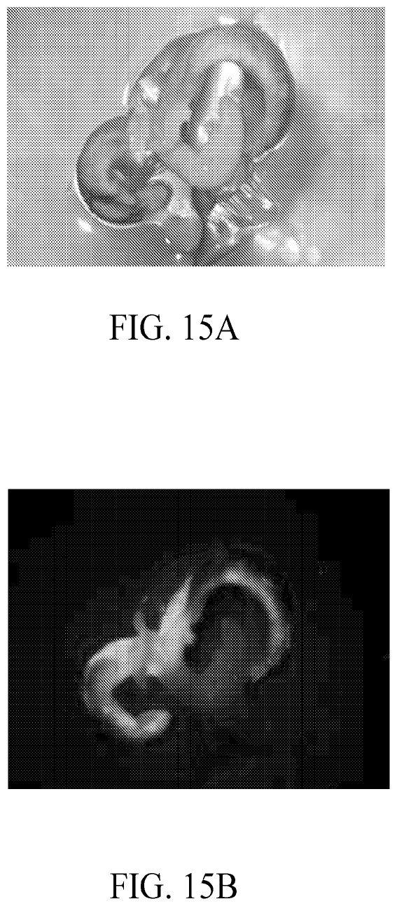

FIGS. 15A-15B are images of a cannabis seedling explant stably transformed with a bacterium strain, consistent with the present disclosure. In some embodiments, cannabis seedlings explants were transformed with plasmid vector 560 as illustrated by FIG. 5B. The seedling explants were stably transformed the transformation protocol as described above and using Agrobacterium containing a binary vector. The binary vector contained different elements including kanamycin selection marker and a YFP reporter (e.g., a visual selection marker). High levels of YFP fluorescence were identified at the meristematic region 7 days after transformation. FIG. 15A is an image of a cannabis explant that was transformed with plasmid vector 560 under white light. FIG. 15B is an image of the cannabis explant of FIG. 15A placed under YFP fluorescence light. The explant shows stable YFP fluorescence at the meristematic region due to integration of the vector into the cannabis genome.

Various experiments were directed to testing different culture media and different concentrations of components of the culture media, such as SIMs with different TDZ concentrations. Cannabis is recalcitrant and in some experiments, the resulting explants did not show transformed shoots. Based on various experiments, it is believed that cannabis plants have strong apical dominance, and breaking the apical dominance will increase transformation efficiency and result in more transgenic events. Somewhat surprisingly, concentrations of between about 2 mg/L to about 10 mg/L of TDZ was used in the SIM to break the apical dominance and to increase shoots in the meristematic cannabis cells. Some experiments were directed to testing different SIMs on cannabis EAs and with different light conditions. For example, the seeds were sterilized and imbibed, and the EAs were extracted. In some experiments, the EAs were not transformed, and between 40 and 50 EAs were regenerated on each different SIMs. In further experiments, a subset of the EAs were plated on an SIM without plant growth regulators, such as TDZ, and the remaining EAs were plated on an SIM with 10 mg/L TDZ, with different groups of each subset being exposed to different light conditions. Tables 2 and 3 summarize example media and light conditions:

TABLE 2
Media

| Week | Day 1 (Wednesday) | | Day 2 (Thursday) | |
|---|---|---|---|---|
| 1 | 1 mg/L TDZ | | 2.5 mg/L TDZ | |
| 2 | 2 mg/L TDZ + | 2 mg/L TDZ + | Glucose + | Glucose + |
| | 0.5 mg/L IAA | 0.5 mg/L IAA + | 2 mg/L TDZ + | 2 mg/L TDZ + |
| | | 40 mg/L AH40 | 0.5 mg/L IAA | 0.5 mg/L IAA + |
| | | | | 40 mg/L AH40 |
| 3 | 2 mg/L TDZ + | 2 mg/L TDZ + | Glucose + | Glucose + |
| | 0.25 mg/L IAA | 0.25 mg/L IAA + | 2 mg/L TDZ + | 2 mg/L TDZ + |
| | | 40 mg/L AH40 | 0.25 mg/L IAA | 0.25 mg/L IAA + |
| | | | | 40 mg/L AH40 |

TABLE 3
Light Conditions

| Light I | Light II | Light III |
|---|---|---|
| Conditions | Conditions | Conditions |
| (B23:G16:R59, | (B15:G7:R78, | (B34:G21:R49, |
| 50 umol) | 90 umol) | 70 umol) |
| No plant growth | -PGRs | -PGRs |
| regulators | | |
| (-PGRs) | | |
| 10 mg/L TDZ | 10 mg/L TDZ | 10 mg/L TDZ |

The B, G, R values in Table 3 include blue, green, and red light values, along with the intensity of the light.

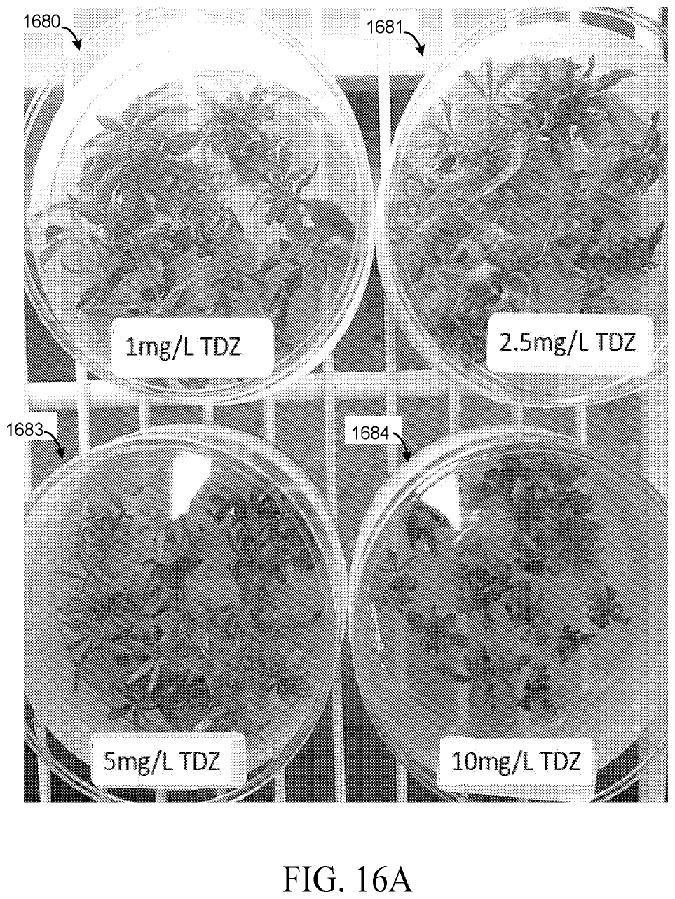

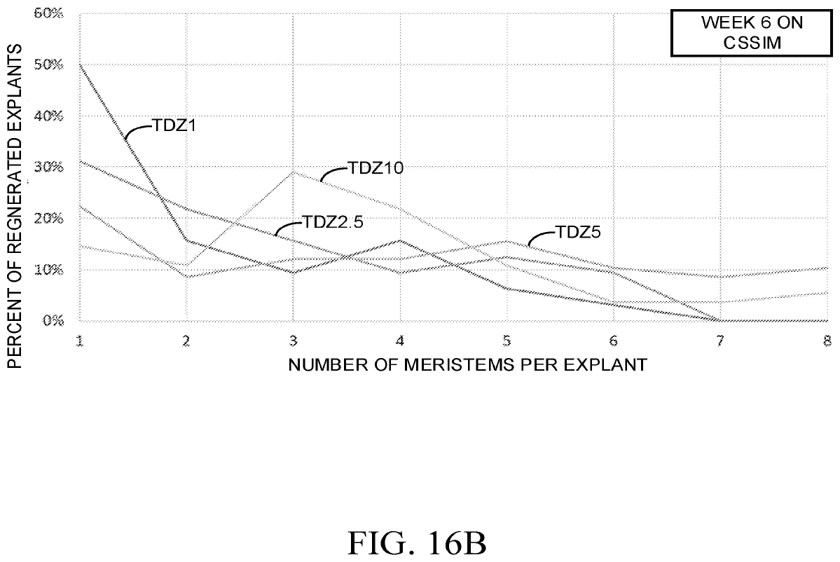

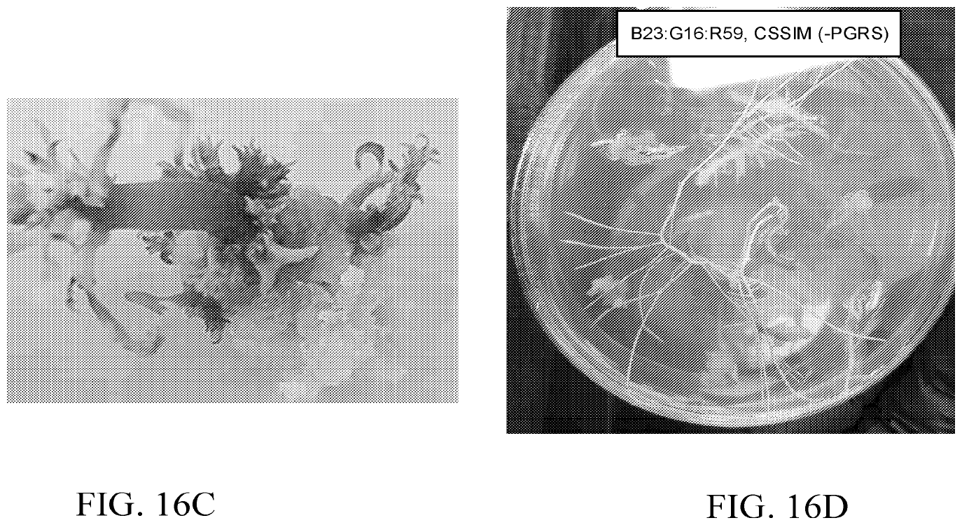

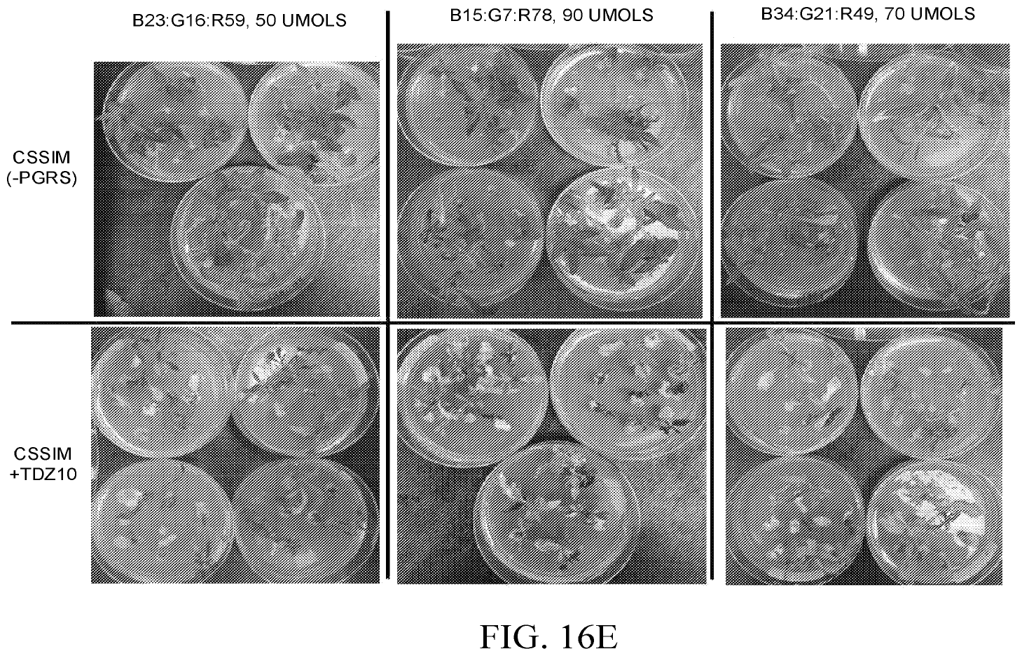

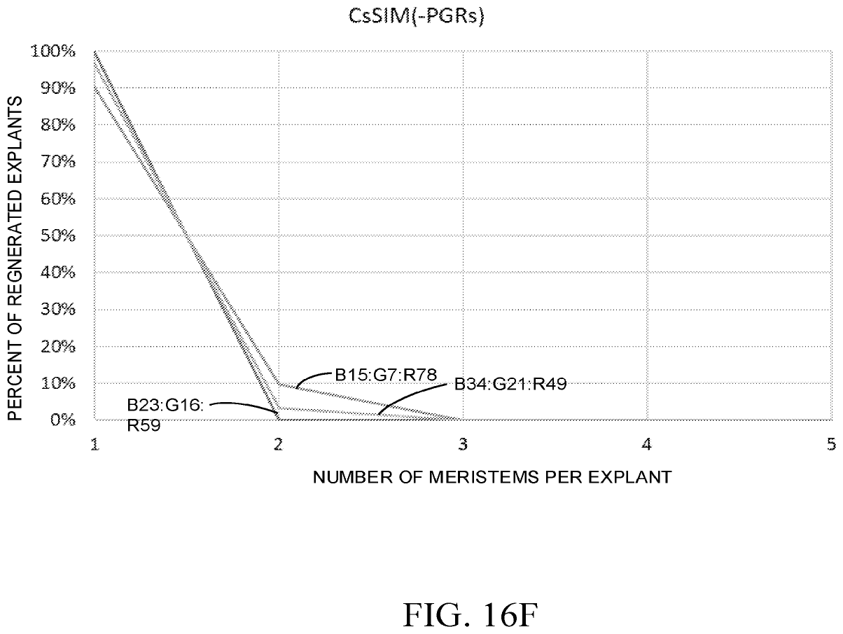

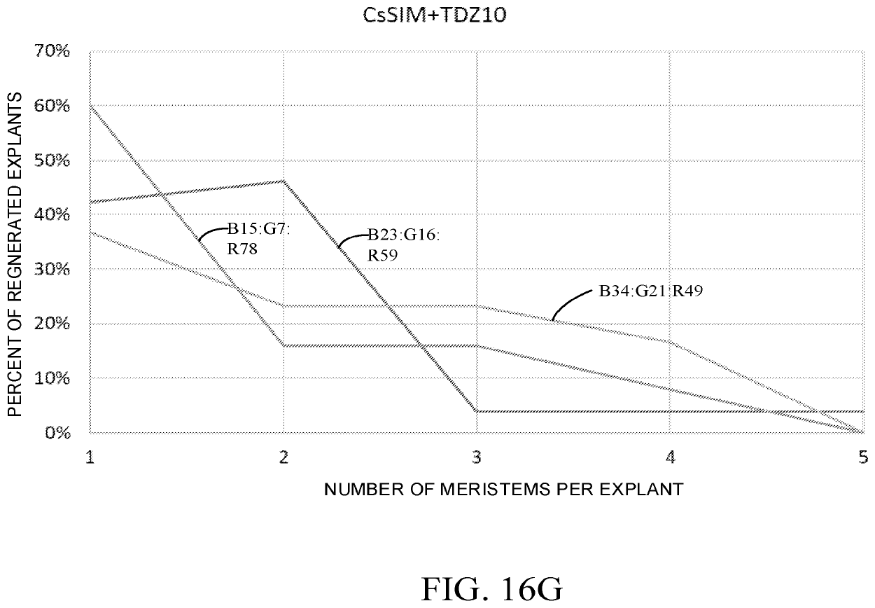

FIGS. 16A-16G are data results of experiments assessing the different culture media and light conditions, consistent with the present disclosure.

FIG. 16A is images 1680, 1681, 1683, 1684 of cannabis explants regenerated from cannabis EAs cultured on different SIMs. Although not illustrated, IAA, Adenine Hemisulfate, and glucose did not have a significant positive impact. The images 1680, 1681, 1683, 1684 show resulting explants regenerated in SIM with 1 mg/L TDZ, 2.5 mg/L TDZ, 5 mg/L TDZ, and 10 mg/L TDZ, respectively. After around three weeks, explants on SIM with 10 mg/L TDZ showed the greatest shoot growth. After around six weeks, explants on SIM with 10 mg/L TDZ showed signs of stress, e.g., chlorosis, callus formation, stunted growth. Accordingly, cannabis EAs can be cultured on SIM with 10 mg/L TDZ for between about 2 to about 6 weeks, and in some embodiments, between about 2 weeks and 3 weeks.

FIG. 16B is a graph illustrating the explants regenerated in the different SIMs as previously described by FIG. 16A and the number of resulting meristems. As shown, the SIM with 10 mg/L TDZ had the most explants with multiple meristems. The SIM with 5 mg/L TDZ had the most explants with a high number of meristems.

FIG. 16C is an image of a resulting explant which was regenerated on SIM containing 10 mg/L TDZ.

Examples are not limited to the media containing TDZ and/or the TDZ concentrations illustrated by FIGS. 16B-16C. In some experiments, additional TDZ concentrations were tested and different combinations of components in the SIM were tested. Tables 4 and 5 illustrate different SIMs tested and resulting meristems at week 2 and week 6.

TABLE 4
Week 2 Results

| | % | % | % | % | % | % |
|---|---|---|---|---|---|---|
| | Explants | Explants | Explants | Explants | Explants | Explants |
| | with 1 | with 2 | with 3 | with 4 | with 5 | with 6 |
| SIM Media | Meristem | Meristem | Meristem | Meristem | Meristem | Meristem |
| SIM + TDZ1 | 39% | 26% | 6% | 29% | 0% | 0% |
| SIM + TDZ2.5 | 61% | 18% | 9% | 12% | 0% | 0% |
| SIM + TDZ1 | 44% | 33% | 0% | 22% | 0% | 0% |
| (SIM + S10 first) | | | | | | |
| SIM + TDZ2.5 | 46% | 15% | 15% | 23% | 0% | 0% |
| (SIM + S10 first) | | | | | | |
| SIM + TDZ5 | 68% | 23% | 5% | 3% | 0% | 0% |
| SIM + TDZ10 | 71% | 14% | 13% | 2% | 0% | 0% |
| SIM + TDZ2 + IAA.5 | 56% | 18% | 10% | 13% | 3% | 0% |
| SIM + TDZ2 + IAA.5 + | 73% | 5% | 16% | 5% | 0% | 0% |
| AH40 | | | | | | |
| GlucoseSIM + TDZ2 + | 71% | 5% | 22% | 2% | 0% | 0% |
| IAA.5 | | | | | | |
| GlucoseSIM + TDZ2 + | 81% | 9% | 5% | 5% | 0% | 0% |
| IAA.5 + AH40 | | | | | | |
| SIM + TDZ2 + IAA.25 | 100% | 0% | 0% | 0% | 0% | 0% |
| (S10) | | | | | | |
| SIM + TDZ2 + IAA.25 + | 100% | 0% | 0% | 0% | 0% | 0% |
| AH40 (S10) | | | | | | |
| GlucoseSIM + TDZ2 + | 100% | 0% | 0% | 0% | 0% | 0% |
| IAA.25 (S10) | | | | | | |
| GlucoseSIM + TDZ2 + | 100% | 0% | 0% | 0% | 0% | 0% |
| IAA.25 + AH40 (S10) | | | | | | |

TABLE 5
Week 6 Results

| | % | % | % | % | % | % |
|---|---|---|---|---|---|---|
| | Explants | Explants | Explants | Explants | Explants | Explants |
| | with 1 | with 2 | with 3 | with 4 | with 5 | with 6 |
| SIM Media | Meristem | Meristem | Meristem | Meristem | Meristem | Meristem |
| SIM + TDZ1 | 50% | 16% | 9% | 16% | 6% | 3% |
| SIM + TDZ2.5 | 31% | 22% | 16% | 9% | 13% | 9% |
| SIM + TDZ1 | 50% | 20% | 20% | 10% | 0% | 0% |
| (SIM + S10 first) | | | | | | |
| SIM + TDZ2.5 | 30% | 10% | 0% | 0% | 0% | 0% |
| (SIM + S10 first) | | | | | | |
| SIM + TDZ5 | 22% | 9% | 12% | 12% | 16% | 10% |
| SIM + TDZ10 | 15% | 11% | 29% | 22% | 11% | 4% |
| SIM + TDZ2 + IAA.5 | | | | | | |
| SIM + TDZ2 + IAA.5 + | | | | | | |
| AH40 | | | | | | |
| GlucoseSIM + TDZ2 + | | | | | | |
| IAA.5 | | | | | | |
| GlucoseSIM + TDZ2 + | | | | | | |
| IAA.5 + AH40 | | | | | | |
| SIM + TDZ2 + IAA.25 | | | | | | |
| (S10) | | | | | | |
| SIM + TDZ2 + IAA.25 + | | | | | | |
| AH40 (S10) | | | | | | |
| GlucoseSIM + TDZ2 + | | | | | | |
| IAA.25 (S10) | | | | | | |
| GlucoseSIM + TDZ2 + | | | | | | |
| IAA.25 + AH40 (S10) | | | | | | |

FIG. 16D is an image of resulting growth of cannabis EAs on SIM containing 10 mg/L TDZ and without any plant growth regulator. In particular, the image shows growth under the light I conditions as described in Table 3 above. The medium without plant growth regulators had good root growth, particularly under the light I and light II conditions, and with the light III conditions having significantly longer shoots.

FIG. 16E illustrates images of resulting growth of cannabis EAs in SIMs under the different light conditions (e.g., light I, light II, and light III conditions) without plant growth regulators and with 10 mg/L TDZ.

FIGS. 16F and 16G are graphs illustrating the results of the different light conditions on the number of meristems per explant in cultures without plant growth regulators (FIG. 16F) and with 10 mg/L TDZ (FIG. 16G). As shown, the light conditions alone did not have significant impact on the shoot number, whereas the use of TDZ did. For example, around half of the 10 mg/L TDZ had multiple meristems, with the light II conditions resulting in 40% explants with 3+ meristems, light II conditions resulting in 24% explants with 3+ meristems, and light I conditions resulting in 12% explants with 3+ meristems.

Various experiments were directed to transforming cannabis EAs using the above-described protocols, and include use of infection, CL, and SIM mediums containing TDZ. For examples, the 50-75 EAs were isolated in 20 mL TDZ infection medium+thiols per infection plate. The TDZ infection medium+thiols were removed after isolation was completed. 18r12 with OD600 of around 0.8 was grown with YEP/AB culture medium. The 18r12 was resuspended in the TDZ infection medium+thiols+100 uM Acetosyringone (AS)+0.02% v/v Silwet L-77. 10 mL of the resuspended 18r12 was sonicated for 80 seconds, with zero sonication producing little to no YFP or other transformant expression. Other examples included 20 to 60 seconds sonication. 20 mL of fresh resuspended 18r12 was then added to 10 mL already in the infection plates and inoculated for 30 minutes to one hour. The 30 mL of the resuspended 18r12 was removed from the infection plates. 5 days of co-cultivation was performed by taking 50-75 EAs from the infection plate and mounding on a piece of sterile paper wetted with 750 μL of ddH₂O in a 100×15 mm petri dish to form co-cultivation plates. The co-cultivation plates were parafilmed and placed in an incubator in conditions including 24 hour ambient light, 0 μmol/m-2/s-1, 40% RH, 23° C. The EAs were then recovered in 100×24 mm petri dish with 20 ml CL medium (0.1% PPM, TDZ), parafilmed, placed in conviron for three days and subcultured daily with 1× sterile ddH₂O rinses in between. The CL medium was removed and the EAs were rinsed with 1× sterile ddH₂O, and placed in SIM+S10 (TDZ2) solid media for around three weeks. Table 6 and 7 below describes the different culture mediums and conditions used to transform the cannabis EAs. In various experiments, the resulting transformed explants were tested using PCR to identify transformation frequencies. Transformed cultures with copy number values between 0.5 and 2.5 were identified and from such cultures, seven transgenic T0s out 47 total T0s were observed, resulting in a resulting transformation efficiency of 14.89%.

TABLE 6

| | Agro- | | | | | | |
|---|---|---|---|---|---|---|---|
| Agro | culture | | Infection | Sonication | Co-Cult | CL medium | SIM |
| vector | medium | OD₆₀₀ | Medium | Time(s) | (5 days) | (3 days) | (18 days) |
| 18r12- | YEP/AB | 0.821 | TDZ | 80 | Mounded, | CL + 0.1% | SIM + S10 |
| plasmid | Min | | | | H20 + FP | PPM + TDZ2 | (TDZ2) |
| vector 570 | | | | | | | |

TABLE 7

| | Agro- | | | | | | | |
|---|---|---|---|---|---|---|---|---|
| Agro | culture | Infection | Sonic. | Infection | Co-Cult | CL medium | SIM | SEM |
| vector | medium | Medium | Time (s) | Strategy | (5 days) | (3 days) | (18 days) | (3 wks) |
| 18r12- | YEP/AB | TDZ + 0.02% | 0 | Ctrl, 1 | Mounded, | CL + 0.1% | SIM + S10 | SEM |
| plasmid | | v/v Silwet | | hour, OD = | 750 uL | PPM + TDZ2 | (TDZ10) | (IAA, GA3) |
| vector 570 | | L-77 | | 0.8-1.0 | H20 + FP | | | |

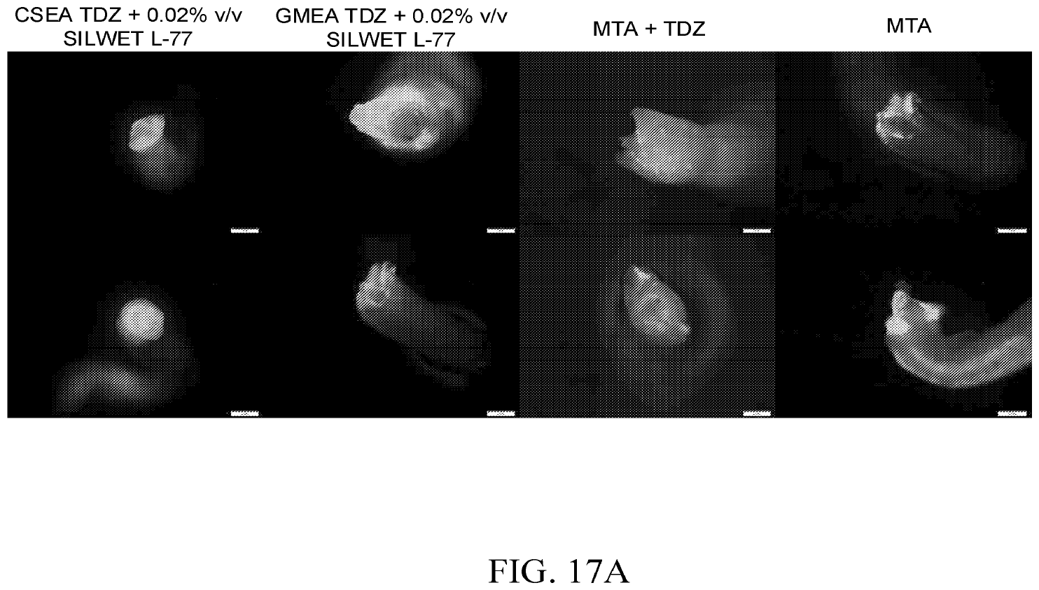

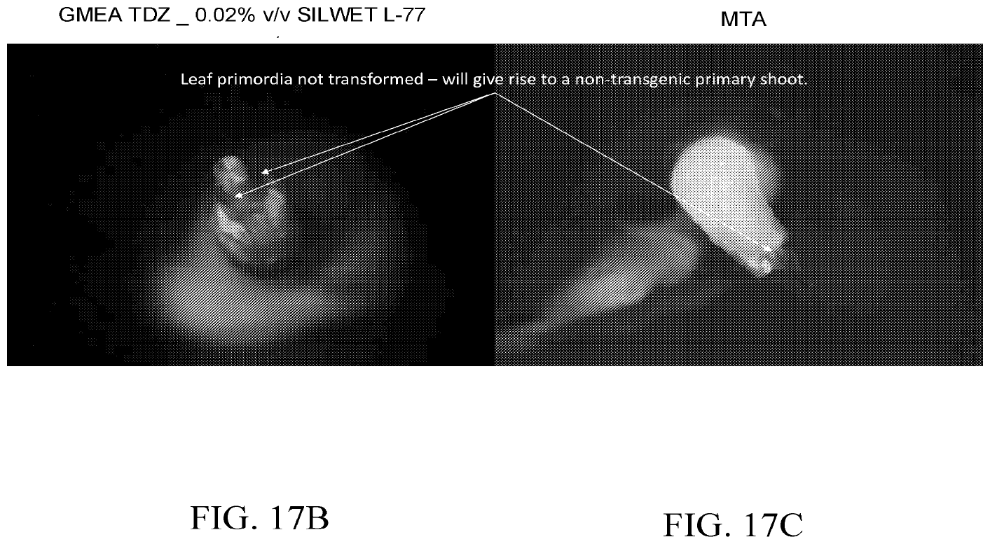

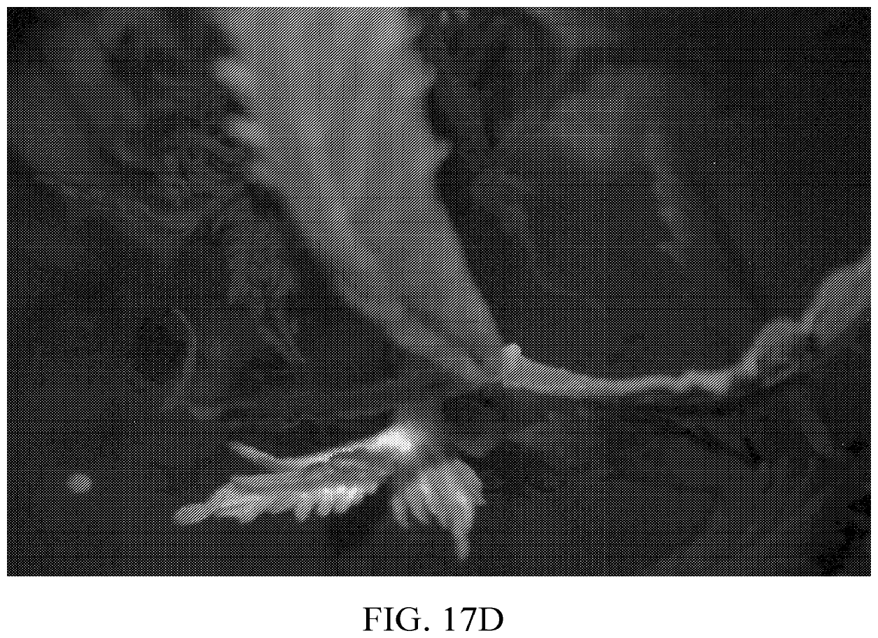

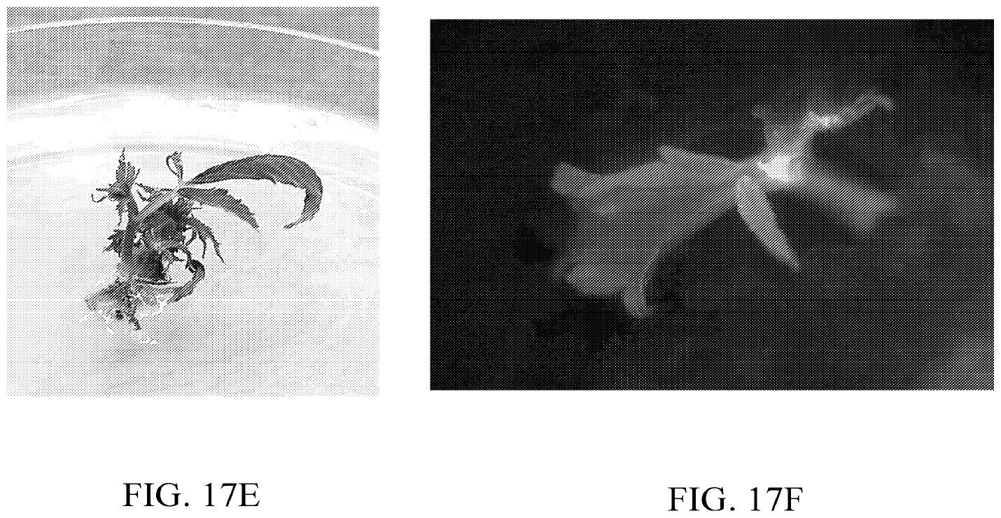

FIGS. 17A-17F are images of explants from experiments assessing the transformation of the explants with different culture media, consistent with the present disclosure. FIG. 17A illustrates images of resulting explants from different infection mediums, including CsEA TDZ+0.02% v/v Silwet L-77, GmEA TDZ+0.02% v/v Silwet L-77 (which is the same as CsEA TDZ+0.02% v/v Silwet L-77 with the salt composition further including gibberellic acid), MTA+TDZ, and MTA. As shown by FIG. 17A, the different infection media treatments resulted in different YFP expression. More particularly, the CsEA TDZ and GmEA TDZ performed between than the MTA+TDZ, and the MTA.

FIGS. 17B-17C illustrates the resulting explants as transformed using GmEA TDZ+0.02% v/v Silwet L-77 (FIG. 17B) and MTA infection media (FIG. 17C). As shown, the leaf primordia is not transformed and will give rise to a non-transgenic primary shoot.

FIGS. 17D-17E are images of a resulting stably transformed explant imaged four weeks after infection with 18r12-plasmid vector 570, and as recovered and regenerated using CL (e.g., 2 mg/L TDZ) and SIM (2 mg/L TDZ).

FIG. 17F is an image of a resulting stably transformed explant imaged five weeks after infection with 18r12-plasmid vector 570, and as recovered and regenerated using CL (e.g., 2 mg/L TDZ) and SIM (2 mg/L TDZ).

In further experimental embodiments, cannabis EAs were transformed with plasmid vectors 572, 576, 578, 580, 582 as illustrated by FIGS. 5D-5I. The cannabis EAs were transformed using the Cannabaceae transformation protocol described above and using bacterium containing a respective binary vector, and regenerating using the above-experimental embodiments steps including the use of the infection medium, CL (with PPM and TDZ2) and SIM (TDZ10). In various embodiments, the editing efficiencies were compared between the different plasmid vectors. The different plasmid vectors included a first set that targeted the PDS gene (e.g., plasmid vector 10, 572 of FIG. 5D, 574 of FIG. 5E, 580 of FIG. 5H) and a second set that targeted the THCAS gene (e.g., plasmid vector 11, 576 of FIG. 5F, 578 of FIG. 5G, 582 of FIG. 5I). Within each of the first set and the second set, respective vectors included no inteins (e.g., plasmid vector 10 and plasmid vector 11 not shown herein), inteins of SSP DnaE and native exteins (e.g., 572 of FIG. 5D, 576 of FIG. 5F), inteins of GP41-1 and non-native exteins (e.g., 574 of FIG. 5E, 578 of FIG. 5G), and inteins of GP41-1 and native exteins (e.g., 580 of FIG. 5H, 582 of FIG. 5I).

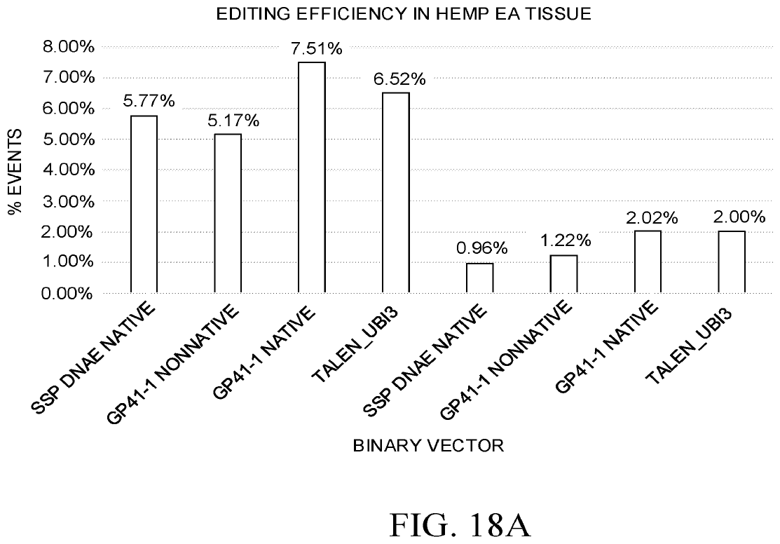

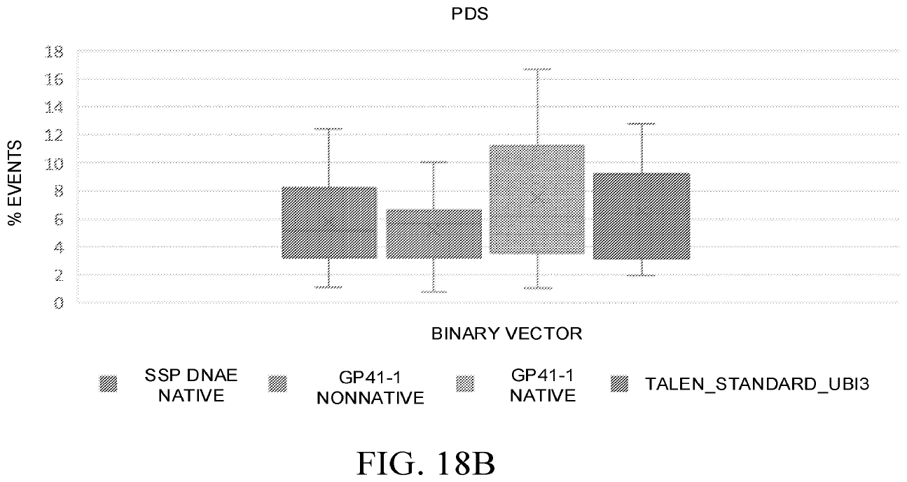

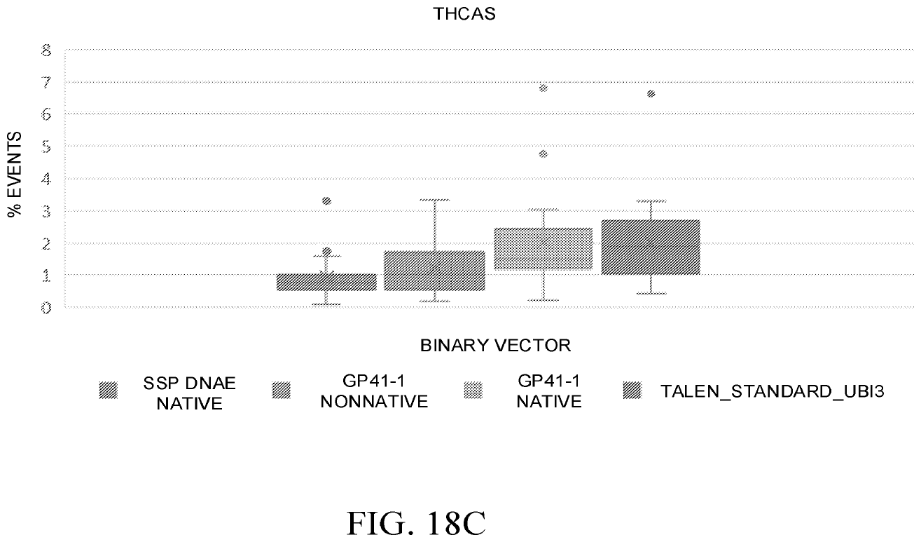

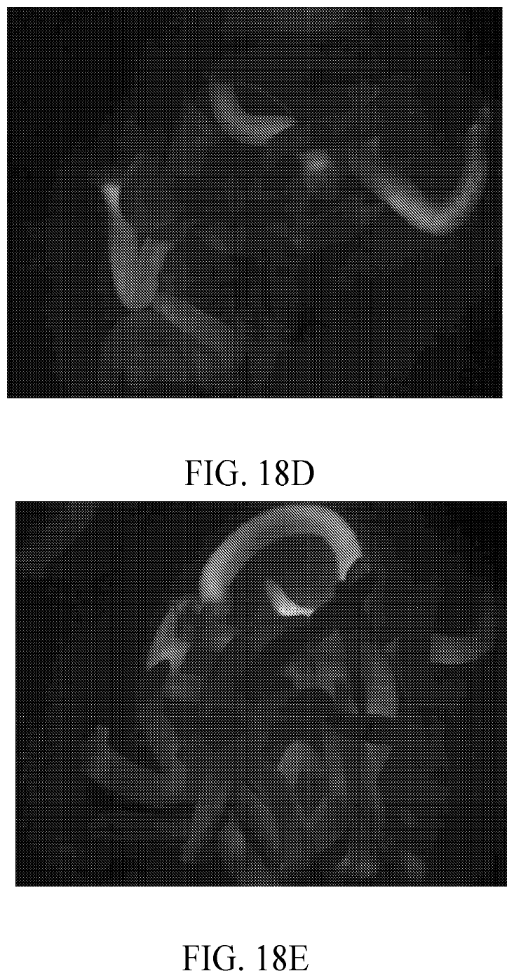

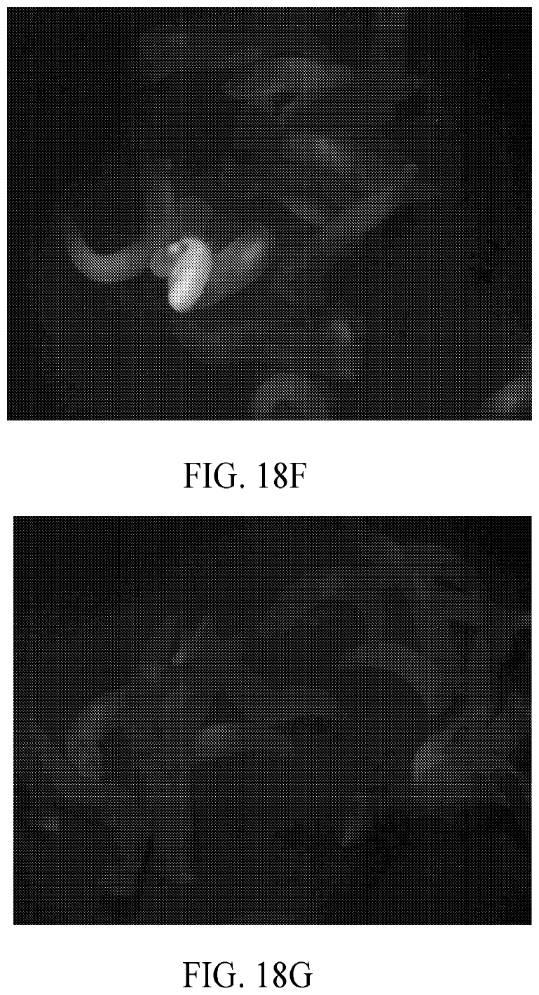

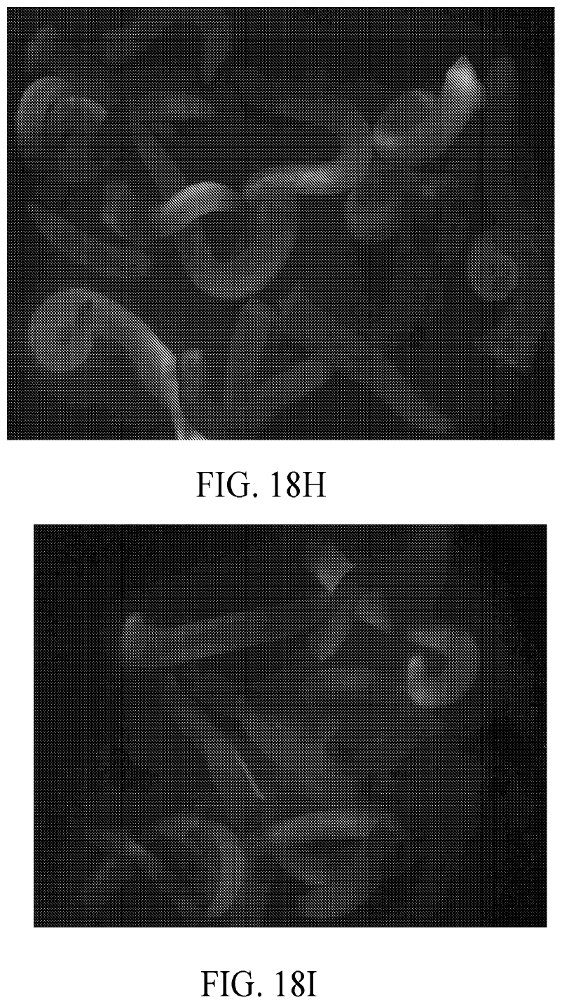

FIGS. 18A-18I illustrate results of transforming explants using the different plasmid vectors, consistent with the present disclosure. FIG. 18A is a graph comparing the editing efficiencies of different plasmid vectors of the first set and second set, illustrating the editing efficiencies of no inteins (e.g., plasmid vector 10 and plasmid vector 11, inteins of SSP DnaE and native exteins (e.g., 572 if FIG. 5D, 574 of FIG. 5F), inteins of GP41-1 and non-native exteins (e.g., 574 of FIG. 5E, 578 of FIG. 5G, and inteins of GP41-1 and native exteins (e.g., 580 of FIG. 5H, 582 of FIG. 5I). FIG. 18B-18C are graph comparing the percent editing events of the different plasmid vectors of the first set and second set, as illustrated by FIG. 18A.

FIG. 18D-18I are images of explants from experiments assessing the transformation of the explants with the different plasmid vectors of FIGS. 5D-5I, consistent with the present disclosure.

The various experimental embodiments were directed to transforming Cannabaceae EAs using various medias. In a number of experiments, the infection medium, CL medium, and SIM medium contained TDZ. Somewhat surprisingly, the SIM medium sometimes contained 2 or 10 mg/L TDZ and resulted in regenerated Cannabaceae explants with transformed cells.

1. A method comprising:
imbibing a Cannabaceae seed in a hydration solution;
excising a subset of embryonic tissue from the imbibed Cannabaceae seed to extract a Cannabaceae meristematic region or embryonic axis (EA); and
exposing the Cannabaceae meristematic region or EA to a heterologous nucleotide sequence to transform Cannabaceae cells of the Cannabaceae meristematic region or EA.

2. The method of claim 1, wherein excising the subset of embryonic tissue comprises removing a seed coat without removing either of the cotyledons of the imbibed Cannabaceae seed.

3. The method of claim 1, wherein excising the subset of embryonic tissue comprises removing a seed coat and one of the cotyledons of the imbibed Cannabaceae seed.

4. The method of claim 1, wherein excising the subset of embryonic tissue comprises removing a seed coat and cutting a radicle of the imbibed Cannabaceae seed.

5. The method of claim 1, wherein excising the subset of embryonic tissue comprises removing a seed coat, both cotyledons, and leaf primordia of the imbibed Cannabaceae seed.

6. The method of claim 1, wherein excising the subset of embryonic tissue comprises removing a seed coat, one of the cotyledons, and leaf primordia of the imbibed Cannabaceae seed.

7. The method of claim 1, further comprising regenerating tissue from the transformed Cannabaceae cells using a culture medium comprising thidiazuron (TDZ), the tissue comprising one or more of shoots, roots, root hair structures, and full plants.

8. The method of claim 7, wherein regenerating the tissue comprises inducing formation of shoots from the transformed Cannabaceae cells using the culture medium.

9. The method of claim 7, wherein the culture medium comprises between about 1 milligram (mg)/liter (L) and about 20 mg/L of TDZ.

10. The method of claim 1, further comprising screening the transformed Cannabaceae cells or tissue regenerated from the transformed Cannabaceae cells for expression of the heterologous nucleotide sequence using a selection agent to screen the transformed Cannabaceae cells or tissue regenerated from the transformed Cannabaceae cells, the selection agent being selected from kanamycin A (kan), g418, spectinomycin, and glyphosate.

11. The method of claim 1, wherein exposing the Cannabaceae meristematic region or EA to the heterologous nucleotide sequence comprises contacting the Cannabaceae meristematic region or EA with a bacterium strain that carries the heterologous nucleotide sequence.

12. The method of claim 11, wherein exposing the Cannabaceae meristematic region or EA to the heterologous nucleotide sequence comprises exposing the Cannabaceae meristematic region or EA to an infection medium comprising the bacterium strain that is transformed to carry the heterologous nucleotide sequence.

13. The method of claim 12, further comprising:
removing the infection medium;
co-culturing the Cannabaceae meristematic region or EA for a threshold period of time; and
culturing the Cannabaceae meristematic region or EA in a selection medium to select transformed Cannabaceae meristematic region or EA.

14. The method of claim 13, wherein the infection medium comprises thidiazuron (TDZ), metolachlor, magnesium sulfate, Tween, acetosyringone (MTA), thiols, GA3, Gamborg's B5 vitamins, DKW salts, AB salts, glucose, Silwet L-77, and combinations thereof.

15. The method of claim 1, wherein the heterologous nucleotide sequence encodes a rare-cutting endonuclease operably connected to a promoter and optionally, a screening marker.

16. A method comprising:
imbibing a Cannabaceae seed in a hydration solution;
excising a subset of embryonic tissue from the imbibed Cannabaceae seed to extract a Cannabaceae meristematic region or embryonic axis (EA);
exposing the Cannabaceae meristematic region or EA to a heterologous nucleotide sequence to transform Cannabaceae cells of the Cannabaceae meristematic region or EA; and
regenerating tissue from the transformed Cannabaceae cells using a culture medium comprising thidiazuron (TDZ).

17. The method of claim 16, wherein the culture medium comprises a shoot inducing medium comprising between about 1 milligram (mg)/liter (L) and about 20 mg/L of TDZ.

18. The method of claim 16, wherein transforming the Cannabaceae cells comprises exposing the Cannabaceae meristematic region or EA to an infection medium comprising a bacterium strain that carries the heterologous nucleotide sequence and that comprises between about 0.1 milligram (mg)/liter (L) and about 2 mg/L of TDZ.

19. The method of claim 16, wherein regenerating the tissue comprises inducing formation of shoots by:
transferring and culturing the Cannabaceae meristematic region or EA in an shoot inducing medium (SIM) comprising the TDZ; and
after culturing in the SIM, transferring and culturing the Cannabaceae meristematic region or EA in a shoot elongation medium (SEM).

20. The method of claim 19, wherein regenerating the tissue further comprises performing recovery by transferring and culturing the Cannabaceae meristematic region or EA in a regeneration medium comprising between about 0.1 (mg)/liter (L) and about 10 mg/L TDZ after the exposure to the heterologous nucleotide sequence and prior to transferring to the SIM, wherein the SIM comprises between about 1 mg/L and about 20 mg/L TDZ.

21-24. (canceled)
